(19) United States
(12) Patent Application Publication
(10) Pub. No.: US 2010/0298342 A1
(43) Pub. Date: Nov. 25, 2010

(54) Spiropiperidine beta-secretase inhibitors for the treatment of Alzheimer's Disease

(75) Inventors: Melissa S. Egbertson, Ambler, PA (US); Shaun R. Stauffer, Schwenskville, PA (US); Craig A. Coburn, Royersford, PA (US); James C. Barrow, Harleysville, PA (US); Lou Anne Neilson, Amity, OR (US); Jenny M. Wai, Harleysville, PA (US); Wenjin Yang, Foster City, CA (US); Wanli Lu, Burlingame, CA (US); Bruce Fahr, Foster City, CA (US)

Correspondence Address: MERCK, P O BOX 2000, RAHWAY, NJ, 07065-0907, US

(73) Assignees: MERCK & CO., INC., Rahway, NJ (US); SUNESIS PHARMACEUTICALS, INC., San Francisco, CA (US)

(21) Appl. No.: 12/310,573

(22) PCT Filed: Sep. 4, 2007
(86) PCT No.: PCT/US2007/019259

§ 371 Date: Aug. 3, 2010

Related U.S. Application Data
(60) Provisional application No. 60/842,728, filed on Sep. 7, 2006.

(51) Int. Cl. A61K 31/438; C07D 471/10; A61K 31/497; A61P 25/28

(52) U.S. Cl. 514/255.05; 546/20; 544/230; 514/278

(57) ABSTRACT

The present invention is directed to spiropiperidine compounds of formula (I) and tautomers thereof, which are inhibitors of the beta-secretase enzyme and that are useful in the treatment of diseases in which the beta-secretase enzyme is involved, such as Alzheimer's disease. The invention is also directed to pharmaceutical compositions comprising these compounds and the use of these compounds and compositions in the treatment of such diseases in which the beta-secretase enzyme is involved.

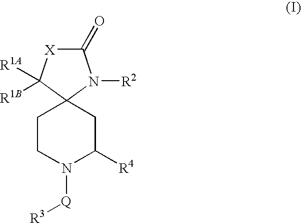

### FIELD OF THE INVENTION

The invention is directed to hydantoin, cyclic urea and cyclic carbamate spiropiperidine compounds which are useful as inhibitors of the beta secretase enzyme, and are useful in the treatment of diseases in which the beta secretase enzyme is involved, such as Alzheimer's Disease.

### BACKGROUND OF THE INVENTION

Alzheimer's disease is the most common cause of dementia in the elderly and is characterized by a decline in cognitive function. Alzheimer's Disease typically progresses slowly and results in symptoms such as memory loss and disorientation. Existing treatments for Alzheimer's Disease, such as acetylcholinesterase inhibitors, address the symptoms of Alzheimer's Disease but do not target the underlying causes of the disease.

The pathology of Alzheimer's disease is characterized by the deposition of amyloid in the brain in the form of extra-cellular plaques and intra-cellular neurofibrillary tangles. The rate of amyloid accumulation is a combination of the rates of formation, aggregation and egress from the brain. It is generally accepted that the main constituent of amyloid plaques is the 4 kD amyloid protein (βA4, also referred to as Aβ, β-protein and βAP) which is a proteolytic product of a precursor protein of much larger size. The amyloid precursor protein (APP or AβPP) has a receptor-like structure with a large ectodomain, a membrane spanning region and a short cytoplasmic tail. The Aβ domain encompasses parts of both extra-cellular and transmembrane domains of APP, thus its release implies the existence of two distinct proteolytic events to generate its NH₂— and COOH-termini.

At least two secretory mechanisms exist which release APP from the membrane and generate soluble, COOH-truncated forms of APP (APPₛ). Proteases that release APP and its fragments from the membrane are termed “secretases.” Most APPₛ is released by a putative α-secretase which cleaves within the Aβ protein to release α-APPₛ and precludes the release of intact Aβ. A minor portion of APPₛ is released by a β-secretase (“β-secretase”), which cleaves near the NH₂-terminus of APP and produces COOH-terminal fragments (CTFs) which contain the whole Aβ domain.

Thus, the activity of β-secretase or β-site amyloid precursor protein-cleaving enzyme (“BACE”) leads to the cleavage of APP, production of Aβ, and accumulation of β amyloid plaques in the brain, which is characteristic of Alzheimer's disease (see R. N. Rosenberg, Arch. Neurol., vol. 59, September 2002, pp. 1367-1368; H. Fukumoto et al, Arch. Neurol., vol. 59, September 2002, pp. 1381-1389; J. T. Huse et al, J. Biol. Chem., vol 277, No. 18, issue of May 3, 2002, pp. 16278-16284; K. C. Chen and W. J. Howe, Biochem. Biophys. Res. Comm, vol. 292, pp 702-708, 2002). Therefore, therapeutic agents that can inhibit 13-secretase or BACE may be useful for the treatment of Alzheimer's disease.

The compounds of the present invention are useful for treating Alzheimer's disease by inhibiting the activity of β-secretase or BACE, thus preventing the formation of insoluble Aβ and arresting the production of Aβ.

### SUMMARY OF THE INVENTION

The present invention is directed to spiropiperidine compounds represented by general formula (I)

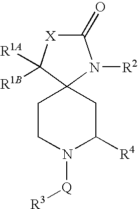

and individual enantiomers and diasteroisomers thereof, and pharmaceutically acceptable salts thereof, which are useful as inhibitors of the β-secretase enzyme.

The invention is also directed to pharmaceutical compositions which include a therapeutically effective amount of a compound of formula (I), or pharmaceutically acceptable salts thereof, and a pharmaceutically acceptable carrier. The invention is also directed to methods of treating mammals for diseases in which the β-secretase enzyme is involved, such as Alzheimer's disease, and the use of the compounds and pharmaceutical compositions of the invention in the treatment of such diseases.

### DETAILED DESCRIPTION OF THE INVENTION

In one embodiment, the present invention is directed to hydantoin, cyclic carbamate and urea spiropiperidine compounds represented by general formula (I)

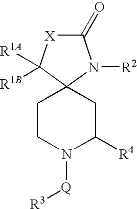

wherein
X is selected from the group consisting of
   (1) N—R⁵,
   (2) O, and
   (3) S,
   and R⁵ is selected from the group consisting of
      (a) hydrogen,
      (b) —C₁₋₁₀ alkyl,
      (c) —C₂₋₁₀ alkenyl,
      (d) —C₃₋₁₂ cycloalkyl,
      (e) —C₀₋₆ alkyl-aryl, and
      (f) —C₀₋₆ alkyl-heteroaryl
   wherein said alkyl, alkenyl, cycloalkyl, aryl and heteroaryl R⁵ moiety is optionally substituted with one or more
      (i) aryl,
      (ii) heteroaryl,
      (iii) halogen,
      (iv) —C₁₋₁₀ alkyl,
      (v) —OC₁₋₁₀ alkyl,
      (vi) —C₃₋₁₂ cycloalkyl,
      (vii) —NC(═O)—R⁶,
      (viii) —C(═O)NR⁶R^{6′},
      (ix) —C(═O)—OR⁶,
      (x) —C(═O)—R⁶,
      (xi) —CN
      (xii) —NR⁶R^{6′},
      wherein said aryl, akly, cycloalkyl and heteroaryl moiety is optionally substituted with one or more
         (I) halogen,
         (II) —C₁₋₆ alkyl,
         (III) —OC₁₋₆ alkyl,
R^{1A} and R^{1B} are each hydrogen, provided that when X is NR⁵, then R^{1A} and R^{1B} may together form ═O;
R² is selected from the group consisting of
   (1) hydrogen,
   (2) —C₁₋₁₀ alkyl,
   (3) —C₂₋₁₀ alkenyl,
   (4) —C₂₋₁₀ alkynyl,
   (5) —C₃₋₁₂ cycloalkyl,
   (6) a heterocyclic group having 4 to 8 ring atoms, wherein one ring atom is a heteroatom selected from the group consisting of nitrogen and oxygen,
   (7) aryl, and
   (8) heteroaryl,
      wherein said alkyl, cycloalkyl, heterocyclic group, alkenyl, alkynyl, aryl or heteroaryl R² moiety is optionally substituted with one or more
      (a) halo,
      (b) —OH,
      (c) —CN,
      (d) —C₁₋₁₀ alkyl,
      (e) —C₂₋₁₀ alkenyl,
      (f) —C₂₋₁₀ alkynyl,
      (g) C₃₋₁₂ cycloalkyl,
      (h) —O—C₁₋₁₀ alkyl,
      (i) —C₀₋₆ alkyl-aryl, or
      (j) —C₀₋₆ alkyl-heteroaryl,
      wherein said alkyl, alkenyl, alkynyl, aryl and heteroaryl moiety is optionally substituted with one or more
         (i) halo,
         (ii) —OH,
         (iii) —CN,
         (iv) —C₁₋₆ alkyl,
         (v) —C₂₋₆ alkenyl,
         (vi) —OC₁₋₆ alkyl,
         (vii) —C₁₋₆ haloalkyl,
         (viii) —SO₂C₁₋₃ alkyl,
         (ix) —SO₂NR⁶R^{6′},
         (x) —CO₂R⁶,
         (xi) —NR⁶SO₂R^{6′},
         (xii) —CONR⁶R^{6′},
         (xiii) —NC(═O)—C₀₋₃ alkyl-NR⁶R^{6′},
         (xiv) —NC(═O)R⁶,
         (xv) —NR⁶R^{6′}, and
         (xvi) a heterocyclic group having 4 to 8 ring atoms, wherein one ring atom is a heteroatom selected from the group consisting of nitrogen and oxygen,
Q is —C₁₋₆ alkylene, wherein said alkylene is optionally substituted with one or more:
   (a) halo,
   (b) —OH,
   (c) —CN,
   (d) —C₁₋₁₀ alkyl
   (e) —C₃₋₁₂ cycloalkyl,
   (f) —O—C₁₋₁₀ alkyl,
   (g) aryl, and
   (h) heteroaryl;
R³ is selected from the group consisting of
   (1) hydrogen,
   (2) —C₁₋₁₀ alkyl,
   (3) —C₂₋₁₀ alkenyl,
   (4) —C₂₋₁₀ alkynyl,
   (5) —C₃₋₁₂ cycloalkyl,
   (6) —C₃₋₁₂ cycloalkenyl,
   (7) aryl, and
   (8) heteroaryl,
   wherein said alkyl, cycloalkyl, cycloalkenyl, alkenyl, alkynyl or aryl or heteroaryl R³ moiety is optionally substituted with one or more
      (a) halo,
      (b) —OH,
      (c) —CN,
      (d) —C₁₋₁₀ alkyl,
      (e) —C₂₋₁₀ alkenyl,
      (f) —C₃₋₁₂ cycloalkyl,
      (g) —O—C₃₋₁₂ cycloalkyl,
      (h) —O—C₁₋₁₀ alkyl,
      (i) —O—C₃₋₁₂ heterocyclic, wherein said heterocyclic group has from 4 to 8 ring atoms, wherein one ring atom is a heteroatom selected from the group consisting of nitrogen, sulfur and oxygen,
      (j) aryl,
      (k) heteroaryl,
      (l) —NR⁶R^{6′},
         and said alkyl, cycloalkyl, aryl and heteroaryl moiety is optionally substituted with one or more
         (i) halo,
         (ii) —OH,
         (iii) —CN,
         (iv) —C₁₋₁₀ alkyl,
         (v) —OC₁₋₁₀ alkyl, and
         (vi) —NR⁶R^{6′}
         (vii) —C₂₋₆ alkenyl,
         (viii) —C₁₋₆ haloalkyl,
         (ix) —SO₂C₁₋₃ alkyl,
         (x) —SO₂NR⁶R^{6′},
         (xi) —CONR⁶R⁶,
         (xii) NR⁵COR^{5′}, wherein R^{5′} is selected from the same group as R⁵, or
         (xiii) NR⁷SO₂R⁶, wherein R⁷ is selected from the group consisting of
            (A) hydrogen,
            (B) —C₁₋₁₀ alkyl, and
            (C) —C₃₋₄ alkenyl;
R⁴ is selected from the group consisting of
   (1) hydrogen,
   (2) —C₁₋₁₀ alkyl, and
   (3) —C₃₋₄ alkenyl,
   wherein said alkyl or alkenyl R⁴ group is optionally substituted with one or more
      (a) halo,
      (b) —OH
      (c) —C₁₋₆ alkyl,
      (d) —CN,
      (e) —O—C₁₋₁₀ alkyl,
      (f) —NR⁸R⁹, wherein R⁸ and R⁹ are selected from the group consisting of
         (i) hydrogen, and
         (ii) —C₁₋₆ alkyl,
      (g) —S(O)ₙ—C₁₋₆ alkyl, wherein n is 0, 1 or 2,
      (h) —C(═O)—R⁷, wherein R⁷ is selected from the group consisting of
         (i) hydrogen,
         (ii) OH,
         (iii) —C₁₋₆ alkyl, and
         (iv) —OC₁₋₆ alkyl, and
         (v) aryl;
R⁶ and R^{6′} are selected from the group consisting, of
   (1) hydrogen,
   (2) —C₁₋₆ alkyl,
   (3) —C₃₋₇ cycloalkyl,
   (4) —C₁₋₆ haloalkyl,
   (5) —C₀₋₆ alkyl-aryl,
   (6) —C₀₋₆alkyl-heteroaryl,
   (7) halo, and
   (8) a heterocyclic group having 4 to 8 ring atoms, wherein one ring atom is a heteroatom selected from the group consisting of nitrogen and oxygen, wherein said aryl or heteroaryl R⁵ moiety is optionally substituted with one or more
      (a) halo,
      (b) —C₁₋₆ alkyl,
      (c) —O—C₁₋₆ alkyl, and
      (d) —NO₂;
         and pharmaceutically acceptable salts thereof, and individual enantiomers and diastereomers thereof.

In preferred embodiments of the compounds of formula (I), X is NR⁵, wherein R⁵ is preferably hydrogen. Alternatively, R⁵ is selected from the group consisting of optionally substituted
(1) —C₁₋₁₀ alkyl,
(2) —C₂₋₁₀ alkenyl,
(3) —C₃₋₁₂ cycloalkyl,
(4) —C₀₋₆ alkyl-aryl, and
(5) —C₀₋₆ alkyl-heteroaryl.

In preferred embodiments of the compounds of formula (I), R² is phenyl, wherein the phenyl is optionally substituted with one or more
(i) halo,
(ii) —OH,
(iii) —CN,
(iv) —C₁₋₁₀ alkyl, and
(v) phenyl optionally substituted with
   (A) halo,
   (B) —OH,
   (C) —CN,
   (D) —C₁₋₆ alkyl,
   (E) —OC₁₋₆ alkyl,
   (F) —SO₂C₁₋₃ alkyl,
   (G) —SO₂NR⁵R^{5′},
   (H) —NR⁵SO₂C₁₋₃alkyl,
   (I) —CO₂R⁵,
   (J) —CONR⁵R^{5′}, and
   (K) —NR⁵CO₂R^{5′}.

In preferred embodiments of the compounds of formula (I), Q is C₁₋₃ alkylene, most preferably —CH₂—, and R³ is phenyl, wherein the phenyl is optionally substituted with one or more
(A) halo,
(B) —OH,
(C) —CN,
(D) —C₁₋₁₀ alkyl,
(E) —OC₁₋₁₀ alkyl, and
(F) phenyl, optionally substituted with
   (i) —C₁₋₆ alkyl,
   (ii) —OC₁₋₆ alkyl,
   (iii) NR⁵R^{5′}.

In preferred embodiments of the compounds of formula (I), R⁴ is —C₁₋₆ alkyl, most preferably methyl or ethyl.

Within the genus of compounds of formula (I), there is a sub-genus of hydantoin spiropiperidine compounds of formula (II)

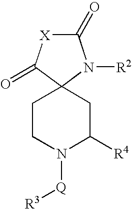

wherein X, Q, R², R³ and R⁴ are as defined above, and pharmaceutically acceptable salts thereof, and individual enantiomers and diastereomers thereof.

In preferred embodiments of the compounds of formula (II), X is NR⁵, and preferably R⁵ is hydrogen. Alternatively, R⁵ is selected from the group consisting of optionally substituted
(1) —C₁₋₁₀ alkyl,
(2) —C₂₋₁₀ alkenyl,
(3) —C₃₋₁₂ cycloalkyl,
(4) —C₀₋₆ alkyl-aryl, and
(5) —C₀₋₆ alkyl-heteroaryl.

In preferred embodiments of the compounds of formula (II), R² is phenyl, wherein the phenyl is optionally substituted with one or more
(i) halo,
(ii) —OH,
(iii) —CN,
(iv) —C₁₋₁₀ alkyl, or
(v) phenyl, optionally substituted with
   (A) halo,
   (B) —OH,
   (C) —CN,
   (D) —C₁₋₆ alkyl,
   (E) —OC₁₋₆ alkyl,
   (F) —SO₂C₁₋₃ alkyl,
   (G) —SO₂NR⁵R^{5′},
   (H) —NR⁵SO₂C₁₋₃alkyl,
   (I) —CO₂R⁵,
   (J) —CONR⁵R^{5′}, and
   (K) —NR⁵COR^{5′}.

In preferred embodiments of the compounds of formula (II), R⁴ is C₁₋₆ alkyl, most preferably methyl or ethyl.

Within the genus of compounds of formula (I), there is a sub-genus of cyclic urea compounds of formula (III)

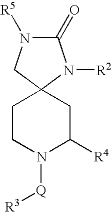

wherein Q, R², R³ , R⁴ and R⁵ are as defined above, and pharmaceutically acceptable salts thereof, and individual enantiomers and diastereomers thereof.

In preferred embodiments of the compounds of formulas (III), Q is C₁₋₃ alkylene, most preferably —CH₂—, and R³ is phenyl, wherein the phenyl is optionally substituted with one or more
(A) halo,
(B) —OH,
(C) —CN,
(D) —C₁₋₁₀ alkyl,
(E) —OC₁₋₁₀ alkyl, or
(F) phenyl, optionally substituted with
   (i) —C₁₋₆ alkyl,
   (ii) —OC₁₋₆ alkyl,
   (iii) NR⁵R^{5′}.

In preferred embodiments of the compounds of formula (III), R⁴ is C₁₋₆ alkyl, most preferably methyl or ethyl.

In preferred embodiments of the compounds of formula (III), R⁵ is selected from the group consisting of
(A) hydrogen,
(B) —C₁₋₁₀ alkyl (preferably —C₁₋₄ alkyl),
(C) —C₂₋₁₀ alkenyl (preferably —C₂₋₄ alkenyl), or
(D) —C₀₋₆ alkyl—aryl (preferably benzyl), or
(E) —C₀₋₆ alkyl-heteroaryl (preferably —CH2-pyridyl).

Within the genus of compounds of formula (I), there is a sub-genus of cyclic carbamate compounds of formula (IV)

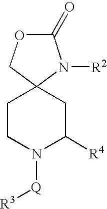

wherein Q, R², R³ and R⁴ and R⁵ are as defined above, and pharmaceutically acceptable salts thereof, and individual enantiomers and diastereomers thereof.

In preferred embodiments of the compounds of formula (IV), Q is C₁₋₃ alkylene, most preferably —CH₂—, and R³ is phenyl, wherein the phenyl is optionally substituted with one or more
(A) halo,
(B) —OH,
(C) —CN,
(D) —C₁₋₁₀ alkyl,
(E) —OC₁₋₁₀ alkyl, or
(F) phenyl, optionally substituted with
   (i) —C₁₋₆ alkyl,
   (ii) —OC₁₋₆ alkyl,
   (iii) NR⁵R^{5′}.

In preferred embodiments of the compounds of formula (IV), R⁴ is C₁₋₆ alkyl, most preferably methyl or ethyl.

In preferred embodiments, the invention is directed to the following exemplary compounds of the invention:
1-(3-fluorophenyl)-8-[(2′-methylbiphenyl-3-yl)methyl]-1,3,8-triazaspiro[4.5]decane-2,4-dione;
4′-fluoro-2′-[8-(3-isopropoxybenzyl)-2,4-dioxo-1,3,8-triazaspiro[4.5]dec-1-yl]-N,N,3-trimethylbiphenyl-4-sulfonamide;
4′-fluoro-2′-[8-(3-isopropoxybenzyl)-2,4-dioxo-1,3,8-triazaspiro[4.5]dec-1-yl]-N,N-dimethylbiphenyl-4-sulfonamide;
4′-fluoro-2′-[8-(3-isopropoxybenzyl)-3-methyl-2,4-dioxo-1,3,8-triazaspiro[4.5]dec-1-yl]-N,N-dimethylbiphenyl-4-sulfonamide;
4′-fluoro-2′-[8-isopropoxybenzyl)-2,4-dioxo-3-propyl-1,3,8-triazaspiro[4.5]dec-1-yl]-N,N-dimethylbiphenyl-4-sulfonamide;
4′-fluoro-2′-[8-(3-isopropoxybenzyl)-2,4-dioxo-1,3,8-triazaspiro[4.5]dec-1-yl]-N,N-dimethylbiphenyl-4-sulfonamide;
2′-(8-benzyl-2,4-dioxo-1,3,8-triazaspiro[4.5]dec-1-yl)-4′-fluoro-N,N-dimethylbiphenyl-4-sulfonamide;
4′-fluoro-2′-[8-(3-isopropoxybenzyl)-2,4-dioxo-1,3,8-triazaspiro[4.5]dec-1-yl]-N,N,3-trimethylbiphenyl-4-carboxamide;
5-{4-fluoro-2-[8-(3-isopropoxybenzyl)-2,4-dioxo-1,3,8-triazaspiro[4.5]dec-1-yl]phenyl}-N,N-dimethylthiophene-2-sulfonamide;
(5R,7S)-8-benzyl-1-(2-bromo-5-fluorophenyl)-7-methyl-1,3,8-triazaspiro[4.5]decane-2,4-dione;
(5R,7S)-1-(2-bromo-5-fluorophenyl)-8-(3-isopropoxybenzyl)-7-methyl-1,3,8-triazaspiro[4.5]decane-2,4-dione;
(5R,7S)-(5S,7R)-1-(3-fluorophenyl)-8-(3-isopropoxybenzyl)-7-methyl-1,3,8-triazaspiro[4.5]decane-2,4-dione;
1-(2-bromo-5-fluorophenyl)-8-(3-isopropoxybenzyl)-1,3,8-triazaspiro[4.5]decane-2,4-dione;
(5R,7S)-(5S,7R)-8-benzyl-1-(3-fluorophenyl)-7-methyl-1,3,8-triazaspiro[4.5]decane-2,4-dione;
N-(3-{4-fluoro-2-[8-(3-isopropoxybenzyl)-2,4-dioxo-1,3,8-triazaspiro[4.5]dec-1yl]phenyl}prop-2-yn-1-yl)-N-methylmethanesulfonamide;
(5R,7S)-1-(3-fluorophenyl)-8-(3-isopropoxybenzyl)-7-methyl-1,3,8-triazaspiro[4.5]decane-2,4-dione;
(5R,7S)-(5S,7R)-8-benzyl-1-[4-fluoro-4′-(methylsulfonyl)biphenyl-2-yl]-7-methyl-1,3,8-triazaspiro[4.5]decane-2,4-dione;
(5R,7S)-1-(3-fluorophenyl)-7-methyl-8-[(2′-methylbiphenyl-3-yl)methyl]-1,3,8-triazaspiro[4.5]decane-2,4-dione;
1-(3-fluorophenyl)-8-[(2′-methylbiphenyl-3-yl)methyl]-3-(pyridin-2-ylmethyl)-1,3,8-triazaspiro[4.5]decan-2-one;
4′-fluoro-2′-[8-(3-isopropoxybenzyl)-2-oxo-1,3,8-triazaspiro[4.5]dec-1-yl]-N,N-dimethylbiphenyl-4-sulfonamide;
4′-fluoro-2′-[8-isopropoxybenzyl)-2-oxo-3-propyl-1,3,8-triazaspiro[4.5]dec-1-yl]-N,N-dimethylbiphenyl-4-sulfonamide;
4′-fluoro-2′-[8-(3-isopropoxybenzyl)-3-methyl-2-oxo-1,3,8-triazaspiro[4.5]dec-1-yl]-N,N-dimethylbiphenyl-4-sulfonamide; 1-[4-fluoro-4′-(methylsulfonyl)biphenyl-2-yl]-8-(3-isopropoxybenzyl)-1,3,8-triazaspiro[4.5]decan-2-one; (5R,7S)-(5S,7R)-8-benzyl-1-(3-fluorophenyl)-7-methyl-1,3,8-triazaspiro[4.5]decan-2-one; (5R,75)-(5 S,7R)-1-(3-fluorophenyl)-7-methyl-8-(3-{[(1R)-1-methylpropyl]oxy}benzyl)-3-pent-4-en-1-yl-1,3,8-triazaspiro[4.5]decan-2-one;
(5R,7S)-1-(3-fluorophenyl)-8-{3-[(1R)-2-methoxy-1-methylethoxy]benzyl}-7-methyl-1,3,8-triazaspiro[4.5]decan-2-one; (5R,7S)-(5S,7R)-8-benzyl-1-[4-fluoro-4′-(methylsulfonyl)-1,1′-biphenyl-2-yl]-7-methyl-1,3,8-triazaspiro[4.5]decane-2,4-dione;
(5R,7S)-1-(3-fluorophenyl)-7-methyl-8-[(2′-methylbiphenyl-3-yl)methyl]-1,3,8-triazaspiro[4.5]decane-2,4-dione;
1-(3-fluorophenyl)-8-[(2′-methylbiphenyl-3-yl)methyl]-3-(pyridin-2-ylmethyl)-1,3,8- triazaspiro[4.5]decan-2-one;
(5R,7S)-8-benzyl-1-(3-fluorophenyl)-7-methyl-3-oxa-1,8-diazaspiro[4.5]decan-2-one; (5R,7S)-1-(3-fluorophenyl)-8-(3-isopropoxybenzyl)-3-[2-(4-methoxyphenyl)ethyl]-7-methyl-1,3,8-triazaspiro[4.5]decane-2,4-dione;
(5R,7S)-3-(cyclohexylmethyl)-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-3-(3-methoxybenzyl)-7-methyl-1,3,8-triazaspiro[4.5]decane-2,4-dione;
N-{2-[(5R,7S)-1-(3-fluorophenyl)-8-(3-isopropoxybenzyl)-7-methyl-2,4-dioxo-1,3,8-triazaspiro[4.5]dec-3-yl]ethyl}benzamide;
(5R,7S)-1-(3-fluorophenyl)-8-(3-isopropoxybenzyl)-7-methyl-3-[(1-methyl-1H-1,2,4-triazol-3-yl)methyl]-1,3,8-triazaspiro[4.5]decane-2,4-dione;
(5R,7S)-1-(3-fluorophenyl)-8-(3-isopropoxybenzyl)-7-methyl-3-[2-(1H-pyrazol-1-yl)ethyl]-1,3,8-triazaspiro[4.5]decane-2,4-dione;
(5R,7S)-1-(3-fluorophenyl)-8-(3-isopropoxybenzyl)-7-methyl-3-[(5-methyl-1,2,4-oxadiazol-3-yl)methyl]-1,3,8-triazaspiro[4.5]decane-2,4-dione;
(5R,7S)-3-(2-fluoroethyl)-1-(3-fluorophenyl)-8-(3-isopropoxybenzyl)-7-methyl-1,3,8-triazaspiro[4.5]decane-2,4-dione;
(5R,7S)-3-(1,2-benzisoxazol-3-ylmethyl)-1-(3-fluorophenyl)-8-(3-isopropoxybenzyl)-7-methyl-1,3,8-triazaspiro[4.5]decane-2,4dione;
(5R,7S)-1-(3-fluorophenyl)-3-[2-(2-fluorophenyl)-2-oxoethyl]-8-(3-isopropoxybenzyl)7-methyl-1,3,8-triazaspiro[4.5]decane-2,4-dione;
(5R,7S)-3-[(1-benzyl-1H-1,2,4-triazol-5-yl)methyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-3-(1H-imidazol-2-ylmethyl)-8-(3-isopropoxybenzyl)-7-methyl-1,3,8-triazaspiro[4.5]decane-2,4-dione;
(5R,7S)-3-{[5-(4-chlorophenyl)-1,3-oxazol-2-yl]methyl}-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-3-[(3-phenyl-1,2,4-oxadiazol-5-yl)methyl]-1,3,8-triazaspiro[4.5]decane-2,4-dione;
(5R,7S)-3-[(5-cyclopropyl-1,3,4-thiadiazol-2-yl)methyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-3-[(5-methylisoxazol-3-yl)methyl]-1,3,8-triazaspiro[4.5]decane-2,4-dione;
(5R,7S)-1-(3-fluorophenyl)-8-(3-isopropoxybenzyl)-7-methyl-3-[(2-methyl-1,3-thiazol-4-yl)methyl]-1,3,8-triazaspiro[4.5]decane-2,4-dione;
(5R,7S)-1-(3-fluorophenyl)-8-(3-isopropoxybenzyl)-3-{[5-(4-methoxyphenyl)-1,2,4-oxadiazol-3-yl]methyl}-7-methyl-1,3,8-triazaspiro[4.5]decane-2,4-dione;
(5R,7S)-3-[(1,3-dimethyl-1H-pyrazol-5-yl)methyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-3-[(5-phenylisoxazol-3-yl)methyl]-1,3,8-triazaspiro[4.5]decane-2,4-dione;
(5R,7S)-3-{[5-(3,4-dichlorophenyl)isoxazol-3-yl]methyl}-1-(3-fluorophenyl)-8-(3-isopropoxybenzyl)-7-methyl-1,3,8-triazaspiro[4.5]decane-2,4-dione;
3-[(5R,7S)-1-(3-fluorophenyl)-8-(3-isopropoxybenzyl)-7-methyl-2,4-dioxo-1,3,8-triazaspiro[4.5]dec-3-yl]propanoic acid;
[(5R,7S)-1-(3-fluorophenyl)-8-(3-isopropoxybenzyl)-7-methyl-2,4-dioxo-1,3,8-triazaspiro[4.5]dec-3-yl]acetic acid;
N-(4-chlorophenyl)-2-[(5R,7S)-1-(3-fluorophenyl)-8-(3-isopropoxybenzyl)-7-methyl-2,4-dioxo-1,3,8-triazaspiro[4.5]dec-3-yl]acetamide;
(5R,7S)-1-(3-fluorophenyl)-8-(3-isopropoxybenzyl)-7-methyl-3-(1,2,4-oxadiazol-3-ylmethyl)-1,3,8-triazaspiro[4.5]decane-2,4-dione;
(5R,7S)-1-(3-fluorophenyl)-8-(3-isopropoxybenzyl)-7-methyl-3-(pyridin-2-ylmethyl)-1,3,8-triazaspiro[4.5]decane-2,4-dione;
(5R,7S)-1-(3-fluorophenyl)-3-(3-furylmethyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-3-(isoxazol-3-ylmethyl)-7-methyl-1,3,8-triazaspiro[4.5]decane-2,4-dione;
(5R,7S)-1-(3-fluorophenyl)-8-(3-isopropoxybenzyl)-7-methyl-3-(1,3-oxazol-2-ylmethyl)-1,3,8-triazaspiro[4.5]decane-2,4-dione;
(5R,7S)-1-(3-fluorophenyl)-8-(3-isopropoxybenzyl)-7-methyl-3-[(3-methyl-1,2,4-oxadiazol-5-yl)methyl]-1,3,8-triazaspiro[4.5]decane-2,4-dione;
(5R,7S)-3-[(3-ethyl-1,2,4-oxadiazol-5-yl)methyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-3-(isoxazol-5-ylmethyl)-7-methyl-1,3,8-triazaspiro[4.5]decane-2,4-dione;
(5R, 7S)- 1-(3-fluorophenyl)-8-(3-isopropoxybenzyl)-7-methyl-3-[(1-methyl-1H-1,2,4-triazol-5-yl)methyl]-1,3,8-triazaspiro[4.5]decane-2,4-dione;
(5R,7S)-1-(3-fluorophenyl)-8-(3-isopropoxybenzyl)-7-methyl-3-[(1-methyl-1H-imidazol-2-yl)methyl]-1,3,8-triazaspiro[4.5]decane-2,4-dione;
(5R,7S)-1-(3-fluorophenyl)-8-(3-isopropoxybenzyl)-7-methyl-3-[(1-phenyl-1H-1,2,3-triazol-4-yl)methyl]-1,3,8-triazaspiro[4.5]decane-2,4-dione;
(5R,7S)-1-(3-fluorophenyl)-8-(3-isopropoxybenzyl)-7-methyl-3-(pyrazin-2-ylmethyl)-1,3,8-triazaspiro[4.5]decane-2,4-dione;
(5R,7S)-3-tert-butyl-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-3-(1-methyl-1-phenylethyl)-1,3,8-triazaspiro[4.5]decane-2,4-dione;
(5R,7S)-1-(3-fluorophenyl)-8-(3-isopropoxybenzyl)-7-methyl-3-(1,3-thiazol-2-ylmethyl)-1,3,8-triazaspiro[4.5]decane-2,4-dione;
(5R,7S)-1-(3-fluorophenyl)-8-(3-isopropoxybenzyl)-7-methyl-3-(1,3-thiazol-4-ylmethyl)-1,3,8-triazaspiro[4.5]decane-2,4-dione;
(5R,7S)-1-(3-fluorophenyl)-8-(3-isopropoxybenzyl)-3-(2-methoxy-1,1-dimethylethyl)-7-methyl-1,3,8-triazaspiro[4.5]decane-2,4-dione;
(5R,7S)-1-(3-fluorophenyl)-3-(2-furylmethyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-2,4-dioxo-3-phenyl-1,3-diaza-8-azoniaspiro[4.5]decane trifluoroacetate;
(5R,7S)-1-(3-fluorophenyl)-8-(3-isopropoxybenzyl)-7-methyl-3-phenyl-1,3,8-triazaspiro[4.5]decane-2,4-dione;
(5R,7S)-1,3-bis(3-fluorophenyl)-8-(3-isopropoxybenzyl)-7-methyl-1,3,8-triazaspiro[4.5]decane-2,4-dione;
3-[(5R,7S)-1-(3-fluorophenyl)-8-(3-isopropoxybenzyl)-7-methyl-2,4-dioxo-1,3,8-triazaspiro[4.5]dec-3-yl]benzonitrile;
(5R,7S)-3-[3-(dimethylamino)phenyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-3-(1H-indol-5-yl)-8-(3-isopropoxybenzyl)-7-methyl-1,3,8-triazaspiro[4.5]decane-2,4-dione;
3-[(5R,7S)-1-(3-fluorophenyl)-8-(3-isopropoxybenzyl)-7-methyl-2,4-dioxo-1,3,8-triazaspiro[4.5]dec-3-yl]benzoic acid;
(5R,7S)-1-(3-fluorophenyl)-8-(3-furylmethyl)-7-methyl-3 -[(5-methylisoxazol-3-yl)methyl]-1,3,8-triazaspiro[4.5]decane-2,4-dione;
N-[4-({(5R,7S)-1-(3-fluorophenyl)-7-methyl-3-[(5-methylisoxazol-3-yl)methyl]-2,4-dioxo-1,3,8-triazaspiro[4.5]dec-8-yl}methyl)phenyl]acetamide;
(5R,7S)-1-(3-fluorophenyl)-7-methyl-3-[(5-methylisoxazol-3-yl)methyl]-8-(pyridin-3-ylmethyl)-1,3,8-triazaspiro[4.5]decane-2,4-dione;
(5R,7S)-8-benzyl-1-(3-fluorophenyl)-7-methyl-3-[(5-methylisoxazol-3-yl)methyl]-1,3,8-triazaspiro[4.5]decane-2,4-dione;
(5R,7S)-8-(2-fluorobenzyl)-1-(3-fluorophenyl)-7-methyl-3-[(5-methylisoxazol-3-yl)methyl]-1,3,8-triazaspiro[4.5]decane-2,4-dione;
(5R,7S)-8-(cyclobutylmethyl)-1-(3-fluorophenyl)-7-methyl-3-[(5-methylisoxazol-3-yl)methyl]-1,3,8-triazaspiro[4.5]decane-2,4-dione;
(5R,7S)-8-benzyl-3- [5-(3,4-dichlorophenyl)isoxazol-3-yl]methyl}-1-(3-fluorophenyl)-7-methyl-1,3,8-triazaspiro[4.5]decane-2,4-dione;
N-(4-{[(5R,7S)-1-(3-fluorophenyl)-7-methyl-2,4-dioxo-1,3,8-triazaspiro[4.5]dec-8-yl]methyl}phenyl]acetamide;
(5R,7S)-3-{[5-(3,4-dichlorophenyl)isoxazol-3-yl]methyl}-8-(2-fluorobenzyl)-1-(3-fluorophenyl)-7-methyl-1,3,8-triazaspiro[4.5]decane-2,4-dione;
(5R,7S)-8-(cyclobutylmethyl)-3-{[5-(3,4-dichlorophenyl)isoxazol-3-yl]methyl}-1-(3-fluorophenyl)-7-methyl-1,3,8-triazaspiro[4.5]decane-2,4-dione;
3-[(5R,7S)-8-(3-isopropoxybenzyl)-7-methyl-2,4-dioxo-1,3,8-triazaspiro[4.5]dec-1-yl]benzonitrile;
3-{(5R,7S)-8-(3-isopropoxybenzyl)-7-methyl-3-[(5-methylisoxazol-3-yl)methyl]-2,4-dioxo-1,3,8-triazaspiro[4.5]dec-1-yl}benzonitrile;
3-[(5R,7S)-3-[(5-cyclopropyl-1,3,4-thiadiazol-2-yl)methyl]-8-(3-isopropoxybenzyl)-7-methyl-2,4-dioxo-1,3,8-triazaspiro[4.5]dec-1-yl]benzonitrile;
3-[(5R,7S)-3-{[5-(3,4-dichlorophenyl)isoxazol-3-yl]methyl}-8-(3-isopropoxybenzyl)-7-methyl-2,4-dioxo-1,3,8-triazaspiro[4.5]dec-1-yl]benzonitrile;
3-{(5R,7S)-8-(3-isopropoxybenzyl)-7-methyl-2,4-dioxo-3-[(1-phenyl-1H-1,2,3-triazol-4-yl)methyl]-1,3,8-triazaspiro[4.5]dec-1-yl}benzonitrile;
3-{(5R,7S)-8-(3-isopropoxybenzyl)-3-[2-(4-methoxyphenyl)ethyl]-7-methyl-2,4-dioxo-1,3,8-triazaspiro[4.5]dec-1-yl}benzonitrile;
N-{2-[(5R,7S)-1-(3-cyanophenyl)-8-(3-isopropoxybenzyl)-7-methyl-2,4-dioxo-1,3,8-triazaspiro[4.5]dec-3-yl]ethyl}benzamide;
3-((5R,7S)-8-(3-isopropoxybenzyl)-3-{[5-(4-methoxyphenyl)isoxazol-3-yl]methyl}-7-methyl-2,4-dioxo-1,3,8-triazaspiro[4.5]dec-1-yl)benzonitrile; and
(5R,7S)-1-(3-fluorophenyl)-7-methyl-8-[(2′-methylbiphenyl-3-yl)methyl]-3-[(5-methylisoxazol-3-yl)methyl]-1,3,8-triazaspiro[4.5]decane-2,4-dione;
and pharmaceutically acceptable salts thereof, and individual enantiomers and diastereomers thereof.

The invention is also directed to methods of treating a patient (preferably a human) for diseases in which the (3-secretase enzyme is involved, such as Alzheimer's disease, by administering a therapeutically effective amount of a spiropiperidine compound of any of the embodiments of formula (I), (II), (III), or (IV), or a pharmaceutically acceptable salt thereof; and a pharmaceutically acceptable carrier.

The invention is also directed to methods of inhibiting BACE1 enzyme activity, by administering a therapeutically effective amount of a spiropiperidine compound of any of the embodiments of formula (I), (II), (III), or (IV), or a pharmaceutically acceptable salt thereof, and a pharmaceutically acceptable carrier, to a mammal or patient in need thereof. In another embodiment, the invention is directed to methods of inhibiting BACE2 enzyme activity, by administering a therapeutically effective amount of a spiropiperidine compound of any of the embodiments of formula (I), (II), (III), or (IV), or a pharmaceutically acceptable salt thereof; and a pharmaceutically acceptable carrier, to a mammal or patient in need thereof.

The invention is also directed to methods of treating a patient (preferably a human) for diseases in which the β-secretase enzyme is involved, such as Alzheimer's disease, by administering a therapeutically effective amount of a spiropiperidine compound of any of the embodiments of formula (I), (II), (III), or (IV), or a pharmaceutically acceptable salt thereof, in combination with a P450 inhibitor, such as ritonavir, and a pharmaceutically acceptable carrier.

The invention is also directed to pharmaceutical compositions for the treatment of diseases in a patient (preferably a human) in which the β-secretase enzyme is involved, such as Alzheimer's Disease, which include a therapeutically effective amount of a compound of any of the embodiments of formula (I), (II), (III), or (IV), or a pharmaceutically acceptable salt thereof, and a pharmaceutically acceptable carrier.

The invention is also directed to pharmaceutical compositions for the treatment of diseases in mammals (preferably humans) in which the β-secretase enzyme is involved, such as Alzheimer's Disease, which include a therapeutically effective amount of a compound of any of the embodiments of formula (I), (II), (III), or (IV), or a pharmaceutically acceptable salt thereof; together with a P450 inhibitor, such as ritonavir, and a pharmaceutically acceptable carrier.

The invention is further directed to a method for the manufacture of a medicament or a composition for inhibiting β-secretase enzyme activity in mammals (preferably humans) and animals comprising combining a therapeutically effective amount of a compound of any of the embodiments of formula (I), (II), (III), or (IV), or a pharmaceutically acceptable salt thereof; with a pharmaceutically acceptable carrier.

The invention is also directed to a method for the manufacture of a medicament or a composition for treating diseases in which the β-secretase enzyme is involved, such as Alzheimer's Disease, in mammals (preferably humans), comprising combining a therapeutically effective amount of compound of any of the embodiments of formula (I), (II), (III), or (IV), or a pharmaceutically acceptable salt thereof; with a pharmaceutically acceptable carrier.

The invention is also directed to a method for the manufacture of a medicament or a composition for treating diseases in which the β-secretase enzyme is involved, such as Alzheimer's Disease, in mammals (preferably humans), comprising combining a compound of any of the embodiments of formula (I), (II), (III), or (IV), or a pharmaceutically acceptable salt thereof, and a P450 inhibitor, such as ritonavir, with a pharmaceutically acceptable carrier.

As used herein, the term “alkyl,” by itself or as part of another substituent, means a saturated straight or branched chain hydrocarbon radical having the number of carbon atoms designated (e.g., C₁₋₁₀ alkyl means an alkyl group having from one to ten carbon atoms). Preferred alkyl groups for use in the invention are C₁₋₆ alkyl groups, having from one to six carbon atoms. Exemplary alkyl groups include methyl, ethyl, n-propyl, isopropyl, n-butyl, isobutyl, tert-butyl, pentyl, hexyl, and the like.

The term “C₀ alkyl,” for example in the term “—C₀alkyl-C₆₋₁₂ aryl”, refers to a bond.

As used herein, the term “alkylene,” by itself or as part of another substituent, means a saturated straight or branched chain divalent hydrocarbon radical having the number of carbon atoms designated.

As used herein, the term “alkenyl,” by itself or as part of another substituent, means a saturated straight chain divalent hydrocarbon radical having the number of carbon atoms designated.

As used herein, the term “alkenyl,” by itself or as part of another substituent, means a straight or branched chain hydrocarbon radical having a single carbon-carbon double bond and the number of carbon atoms designated (e.g., C₂₋₁₀ alkenyl means an alkenyl group having from two to ten carbon atoms). Preferred alkenyl groups for use in the invention are C₂₋₆ alkenyl groups, having from two to six carbon atoms. Exemplary alkenyl groups include ethenyl and propenyl.

As used herein, the term “alkynyl,” by itself or as part of another substituent, means a straight or branched chain hydrocarbon radical having a single carbon-carbon triple bond and the number of carbon atoms designated (e.g., C₂₋₁₀ alkynyl means an alkynyl group having from two to ten carbon atoms). Preferred alkynyl groups for use in the invention are C₂₋₆ alkynyl groups, having from two to six carbon atoms. Exemplary alkynyl groups include ethynyl and propynyl.

As used herein, the term “cycloalkyl,” by itself or as part of another substituent, means a saturated cyclic hydrocarbon radical having the number of carbon atoms designated (e.g., C₃₋₁₂ cycloalkyl means a cycloalkyl group having from three to twelve carbon atoms). The term cycloalkyl as used herein includes mono-, bi- and tricyclic saturated carbocycles, as well as bridged and fused ring carbocycles, such as Spiro fused ring systems.

Preferred cycloalkyl groups for use in the invention are monocyclic C₃₋₈ cycloalkyl groups, having from three to eight carbon atoms. Exemplary monocyclic cycloalkyl groups include cyclopropyl, cyclobutyl, cyclopentyl, cyclohexyl and the like. Exemplary bridged cycloalkyl groups include adamantly and norbornyl. Exemplary fused cycloalkyl groups include decahydronaphthalene.

As used herein, the term “heterocyclic,” by itself or as part of another substituent, means a cycloalkyl group as defined above, in which one or more of the ring carbon atoms is replaced with a heteroatom (such as N or O). Suitable non-aromatic heterocyclic groups for use in the invention include piperidinyl, piperazinyl, morpholinyl, tetrahydropyranyl, tetrahydrofuranyl, pyrrolidinyl, pyrazolidinyl and imidazolildinyl. Preferred heterocyclic groups for use in the invention have four to eight ring atoms and a single nitrogen or oxygen heteroatom.

When a heterocyclic group as defined herein is substituted, the substituent may be bonded to a ring carbon atom of the heterocyclic group, or to a ring heteroatoth (i.e., a nitrogen, oxygen or sulfur), which has a valence which permits substitution. Preferably, the substituent is bonded to a ring carbon atom. Similarly, when a heterocyclic group is defined as a substituent herein, the point of attachment may be at a ring carbon atom of the heterocyclic group, or on a ring heteroatom (i.e., a nitrogen, oxygen or sulfur), which has a valence which permits attachment. Preferably, the attachment is at a ring carbon atom.

As used herein, the term “aryl,” by itself or as part of another substituent, means an aromatic or cyclic radical having the number of carbon atoms designated (e.g., C₆₋₁₀ aryl means an aryl group having from six to ten carbons atoms). The term “aryl” includes multiple ring systems (such as fused ring systems) as well as single ring systems, and includes multiple ring systems wherein part of the molecule is aromatic and part is non-aromatic. The preferred single ring aryl group for use in the invention is phenyl. Preferred fused ring aryl groups include naphthyl, tetrahydronaphthyl and indanyl.

The term “halo” or “halogen” includes fluoro, chloro, bromo and iodo.

As used herein, the term “heteroaryl,” by itself or as part of another substituent, means an aromatic cyclic group having at least one ring heteroatom (O, N or S). The term “heteroaryl” includes multiple ring systems as well as single ring systems. Preferred heteroaryl groups have from 5 to 12 ring atoms. Exemplary heteroaryl groups include pyrazinyl, pyrazolyl, pyridazinyl, pyridyl, pyrimidinyl, pyrrolyl, tetrazolyl, furanyl, imidazolyl, indazolyl, triazinyl, pyranyl, thiazolyl, thienyl, thiophenyl, triazolyl, oxazolyl, isoxazolyl, oxadiazolyl, indolyl, quinolinyl, isoquinolinyl, benzimidazolyl, benzofuranyl and benzoxazolyl. Preferred heteroaryl groups for use in the invention have 5 or 6 ring atoms. Exemplary groups include furanyl, thienyl and pyridyl.

When a heteroaryl group as defined herein is substituted, the substituent may be bonded to a ring carbon atom of the heteroaryl group, or on a ring heteroatom (i.e., a nitrogen, oxygen or sulfur), which has a valence which permits substitution. Preferably, the substituent is bonded to a ring carbon atom. Similarly, when a heteroaryl group is defined as a substituent herein, the point of attachment may be at a ring carbon atom of the heteroaryl group, or on a ring heteroatom (i.e., a nitrogen, oxygen or sulfur), which has a valence which permits attachment. Preferably, the attachment is at a ring carbon atom.

As used herein, the term “beta-secretase” or “β-secretase” refers to an enzyme that is sometimes known in the literature as “BACE”, “BACE1” (see, e.g., Vassar et al., 1999, Science 286:735-741), or “BACE2” (see, e.g., Farzan et al., 2000, PNAS 97:9712-9717). BACE1 is a 501 amino acid membrane-bound aspartic protease. BACE1 has all the known functional properties and characteristics of β-secretase. BACE2, also called Asp-1 or memapsin-1, is a second member of the BACE family of membrane-bound aspartic proteases. See Roggo, Current Topics in Medicinal Chemistry, 2002, 2:359-370, for a further discussion of the differences between BACE1 and BACE2.

The compounds of the invention are inhibitors of both the BACE1 and BACE2 enzyme.

The compounds of the invention have at least two asymmetric centers. Additional asymmetric centers may be present depending upon the nature of the various substituents on the molecule.

For example, for the compounds of formula (I) and (IA), the 5-carbon and 7-carbon of the spiropiperidine ring are chiral. As a result, the compounds of formula (I) and (IA) may be present as two racemic diastereomers, or in four stereochemically pure forms.

The diastereomeric forms for compounds of formula (I) are depicted below, as diastereomers (I′), where the amine of the spiro center and the R⁴ group are cis to one another, and (I″), where the amine of the spiro center and the R⁴ group are trans to one another.

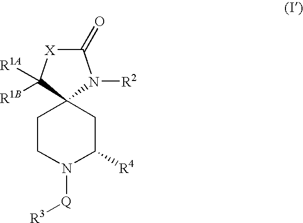

or

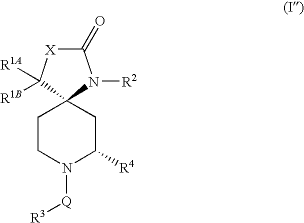

When R⁴ is methyl or ethyl, the diastereomer (I′) is the 5(S,R), 7(S,R) configuration, the diastereomer (I″) is the 5(R,S),7(S,R) configuration.

Each of (I′) and (1″) may be present as a racemic mixture, or in one of two enantiomeric forms, as shown below with compound (I″), as compounds (I″) and (I″*):

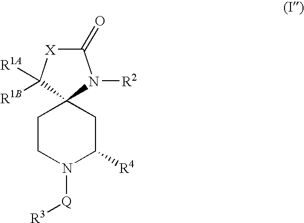

or

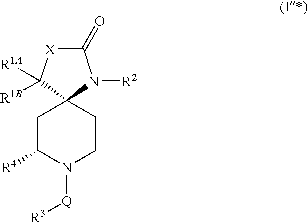

wherein when R⁴ is methyl or ethyl, the I″ enantiomer is the 5(R),7(S) configuration and the I″* enantiomer is the 5(S),7(R) configuration.

Compounds of each of formulas (II), (III) and (IV) are also present in diastereomeric forms. For example, the diastereomeric forms for compounds of formula (II) are depicted below, as diastereomers (II′), where the amine of the spiro center and the R⁴ group are cis to one another, and (II″), where the amine of the spiro center and the R⁴ group are trans to one another.

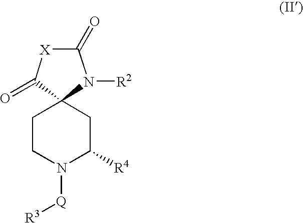

or

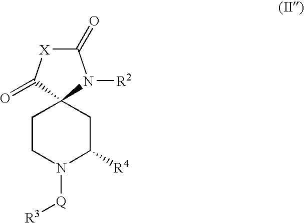

When R⁴ is methyl or ethyl, the diastereomer (II′) is the 5(S,R), 7(S,R) configuration, the diastereomer (II″) is the 5(R,S),7(S,R) configuration.

Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all such isomeric forms of these compounds.

Compounds described herein may contain one or more double bonds, and may thus give rise to cis/trans isomers as well as other configurational isomers. The present invention includes all such possible isomers as well as mixtures of such isomers.

Formulas (I)-(IV) are shown above without a definite stereochemistry at certain positions. The present invention includes all stereoisomers of formulas (I)-(IV), and pharmaceutically acceptable salts thereof.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration.

If desired, racemic mixtures of the compounds may be separated so that the individual enantiomers or diastereomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art.

Alternatively, any enantiomer or diastereomer of a compound may be obtained by stereoselective synthesis using optically pure starting materials or reagents of known configuration by methods well known in the art.

The compounds claimed in this invention can be prepared according to the following general procedure methods.

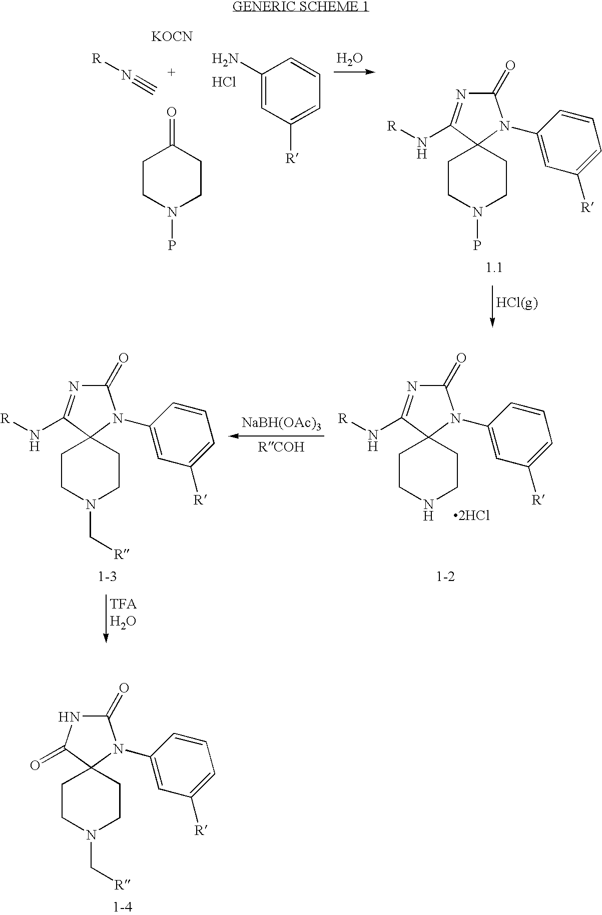

Generic Scheme 1 depicts the formation of compounds of the invention. Combination of an isonitrile, an amine salt, a 4-piperidinone in which the nitrogen is bonded to a removable group P, and a cyanate salt are combined in an Ugi reaction similar to that described in I. Ugi, et. al. Angew. Chem. Int. Ed. Engl. 1, 8-21, 1962 to give the intermediate iminohydantoin 1-1. The protecting group P may then be removed by a suitable method (HCl gas in EtOAc, for example, if the protecting group is BOC) to give the amine salt, which may then be derivatized with a suitable aldehyde through a reductive amination procedure similar to that described in J. March, Advanced Organic Chemistry, 3^{rd} Ed. John Wiley and Sons, NY page 799. Hydrolysis of the iminohydantoin to the hydantoin then may take place under acidic conditions to give the product 1-4.

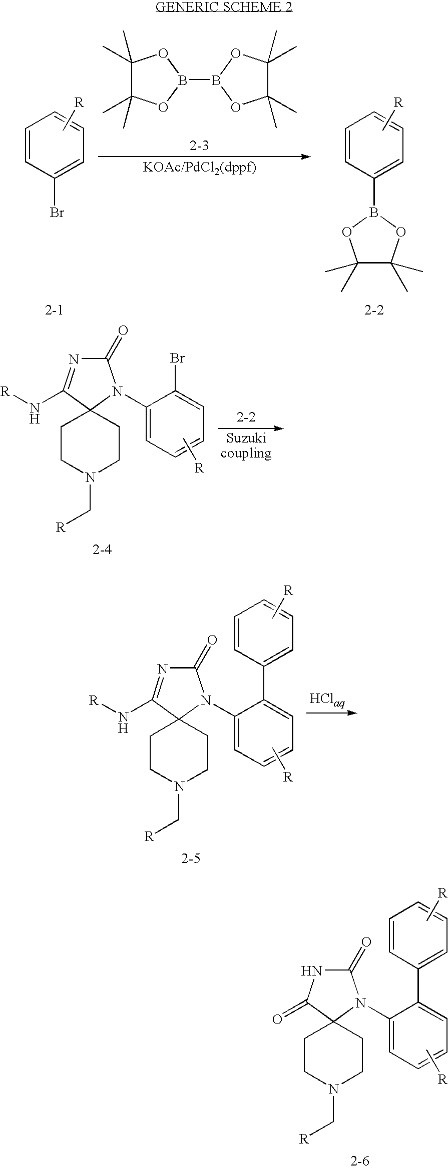

A similar method is outlined in Generic Scheme 2. A suitable boronic ester reagent 2-2 may be prepared and coupled under organometallic catalysis to the iminohydantoin 2-4 to give the biphenyl intermediate 2-5, which is then hydrolyzed to the hydantoin 2-6 as described above.

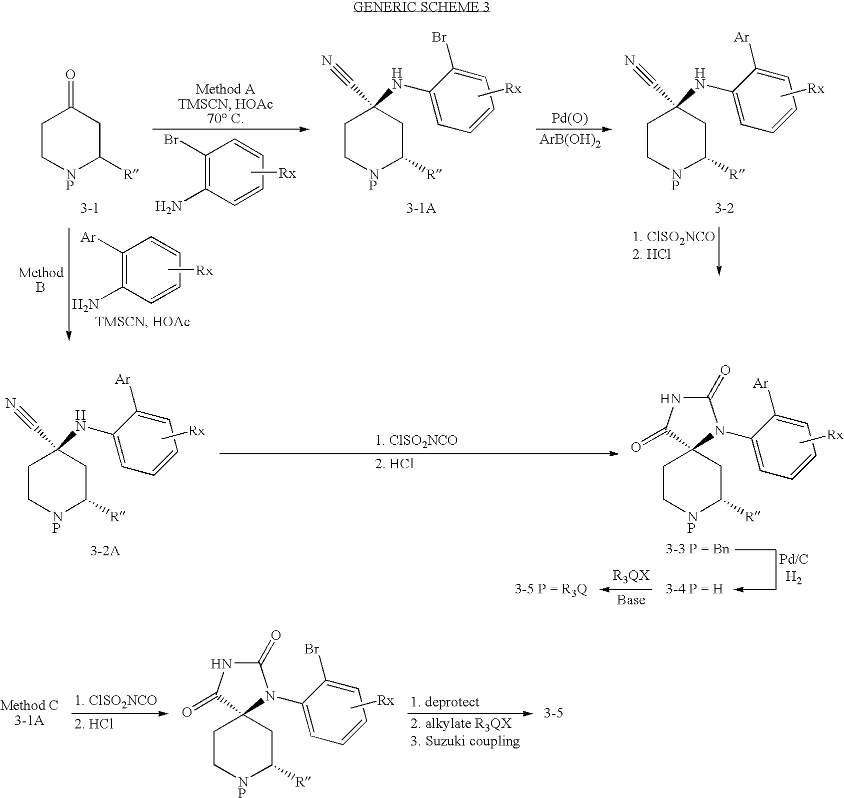

Generic Scheme 3 describes the synthesis of ortho biphenyl examples of type 3-3, 3-4 and 3-5, where R″ may be H or Q (piperidine substituent). One of three methods, which vary on the entry point of the desired biphenyl, may be used to prepare analogs within this structural type. Method A starting from 3-1 from International Patent Application WO 2007/011833 involves first a Strecker reaction (similar to that described by J. Cossy in Synthesis 1995 11 1368-1370) with an o-haloaniline to give 3-1A and its cis isomer which may be separated or taken on together to the next step. A Suzuki coupling and ring closure with chlorosulfonylisocyanate (similar to that described by R. Sarges et al. J Org Chem 1982, 47, 4081-4085) gives 3-3. When P is a readily removable group, for example benzyl or CBZ, P can be removed with a suitable conditions, such as with a catalyst like Pd/C and H₂, to give 3-4. Further elaboration via alkylation or reductive amination gives examples of type 3-5. Method B uses a o-biphenylanilines initially in a Strecker reaction to give 3-2 followed by ring-closure and functional group manipulation as before. Method C starts from 3-1A and begins with ring-closure, then piperidine modification as needed and final Suzuki coupling to give additional examples of type 3-5.

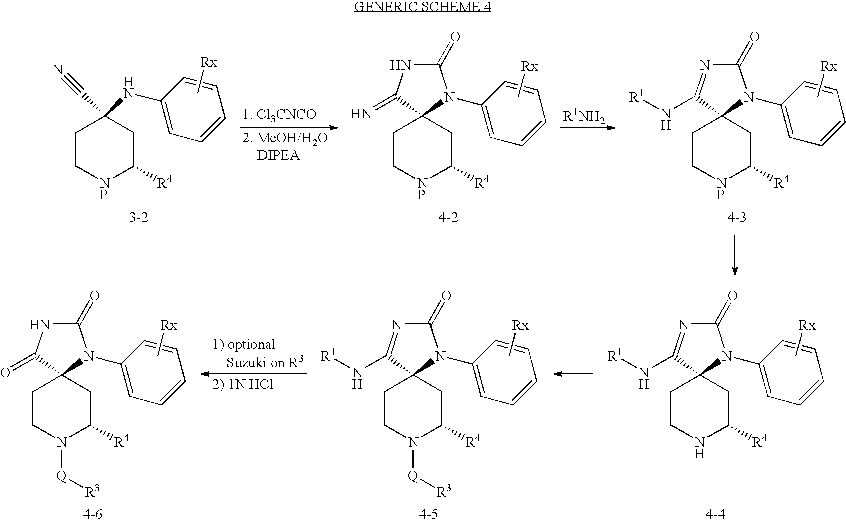

Generic Scheme 4 describes the synthesis of additional examples of the invention. Starting from 3-2 prepared as described above in Generic Scheme 3, the preparation begins with a ring closure with trichloroacetylisocyanate (similar to methods described in International Patent Application WO 2007/011833) to the intermediate iminohydantoin 4-2 which is then reacted with an amine to give the substituted iminohydantoin 4-3. The protecting group is removed with Pd(OH)₂ and H₂ to give 4-4. Further elaboration via alkylation or reductive amination gives examples of type 4-5. Optional Suzuki coupling to further elaborate QR³ can occur using Pd(0) followed by hydrolysis of the iminohydantoin to then give the hydantoin product 4-6.

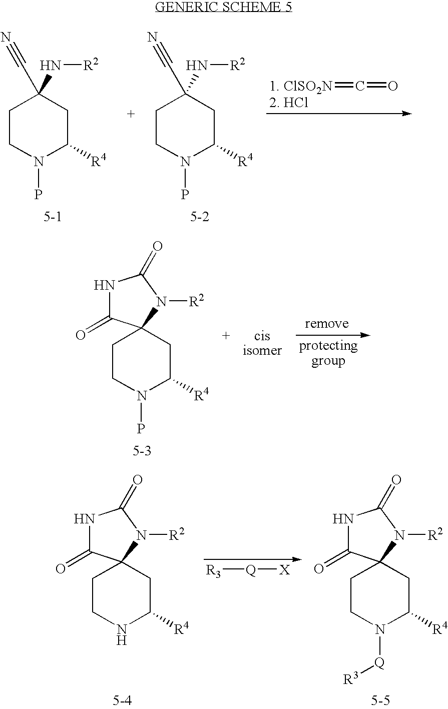

A more direct route similar to Generic Scheme 4 that may be used is described in Generic Scheme 5. The mixture of Strecker products obtained in Scheme 4 may be cyclized in the presence of acid to give the hydantoin product 5-3 directly. The piperidine nitrogen protecting group may then be removed with a suitable method like hydrogenation and the nitrogen then derivatized by a suitable method, such as reductive amination or alkylation, using the methods described above, to give the products 5-5.

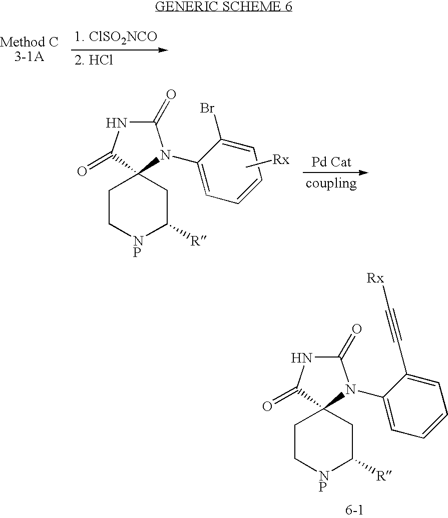

Generic Scheme 6 depicts a variation on generic scheme 3 in which the hydantoin ring formation takes place first and then is followed with the palladium catalyzed coupling of an alkyne to yield compounds of the type 6-1.

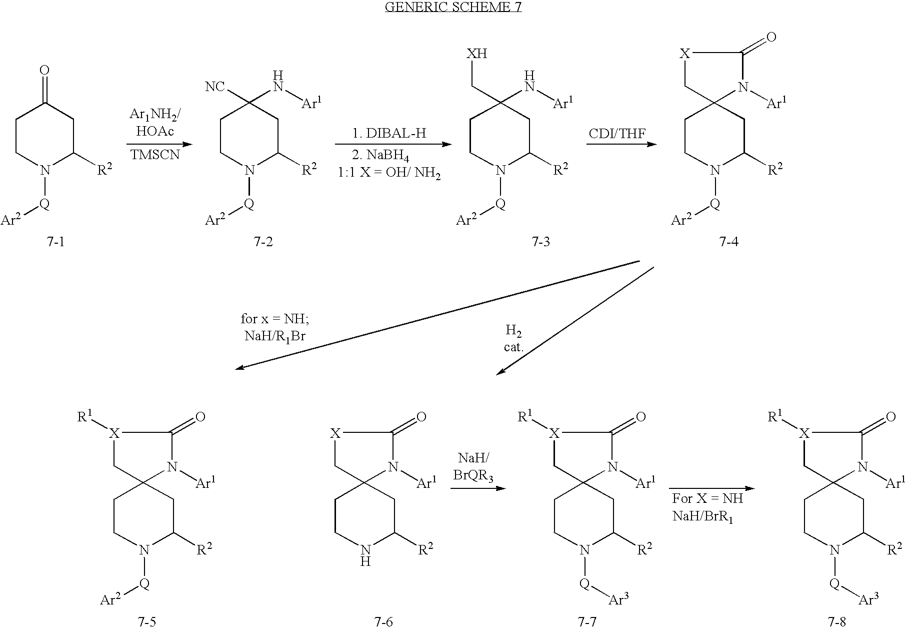

Generic Scheme 7 outlines the preparation of cyclic ureas and carbamates covered by the scope of the invention. The product of the Strecker reaction, prepared in a manner similar to that described in J. Cossy, et. al. Synlett 1998, 251-252 described in Generic Scheme 3 above is reduced with a suitable reducing agent like DIBAL, or NaBH₄, or with a suitable catalyst like Raney Nickel or Rhodium and hydrogen to give the alcohol and amine products, which are cyclized with carbonyldiimidazole to give the cyclic urea or carbamate 7-4. The urea products may then be further functionalized by alkylation with alkyl halides and a suitable base like NaH to give products 7-5, or if QR³ is a removable group like benzyl, it may be removed via reduction with a suitable catalyst and H2 to give products 7-6 which may be alkylated to give products 7-7 and 7-8.

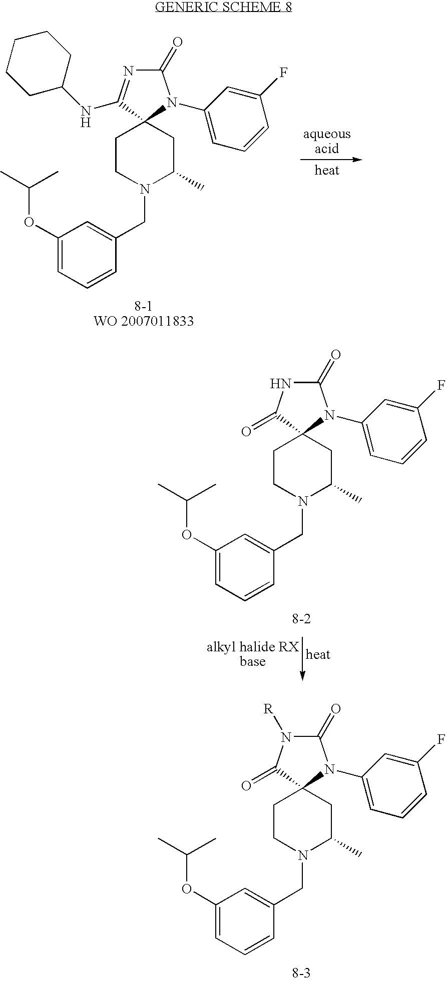

Generic Scheme 8 outlines the preparation of compounds having a variety of substituents on the nitrogen of the hydantoin moiety. The iminohydantoin 8-1, the synthesis of which is described in International patent application WO2007/011833, is treated with aqueous acid and heat to afford the hydantoin 8-2. This material then may be treated with a base like potassium carbonate and an alkylating agent like an alkyl halide in a suitable solvent like DMF to give the alkylated product 8-3.

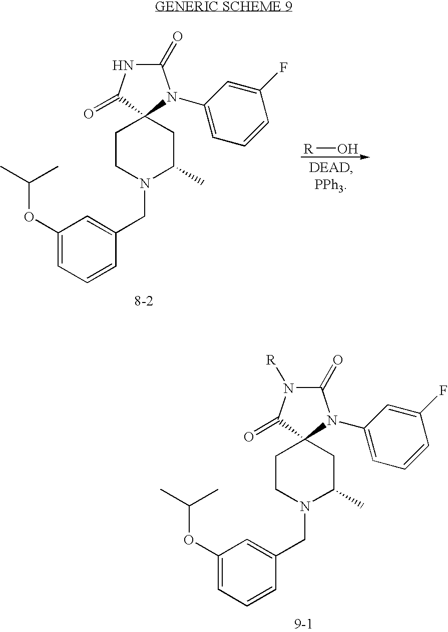

Generic Scheme 9 outlines another method of preparing compounds having a variety of substituents on the nitrogen of the hydantoin moiety. In this method, the hydantoin 8-2 (described in Generic Scheme 8) is treated with a suitable alcohol under Mitsunobu reaction conditions, employing reagents like DEAD and triphenylphosphine to give the products 9-1.

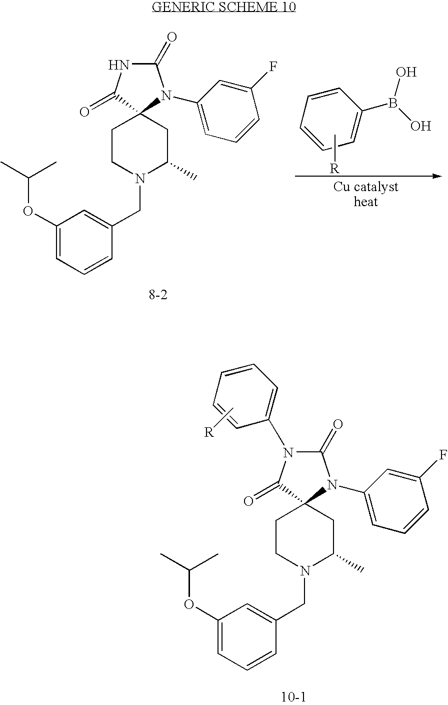

Generic Scheme 10 outlines another method of preparing compounds having a variety of substituents on the nitrogen of the hydantoin moiety. In this method, the hydantoin 8-2 (described in Generic Scheme 8) is treated with a suitable boronic acid under in the presence of a copper catalyst like copper acetate to give the N-aryl analogs 10-1.

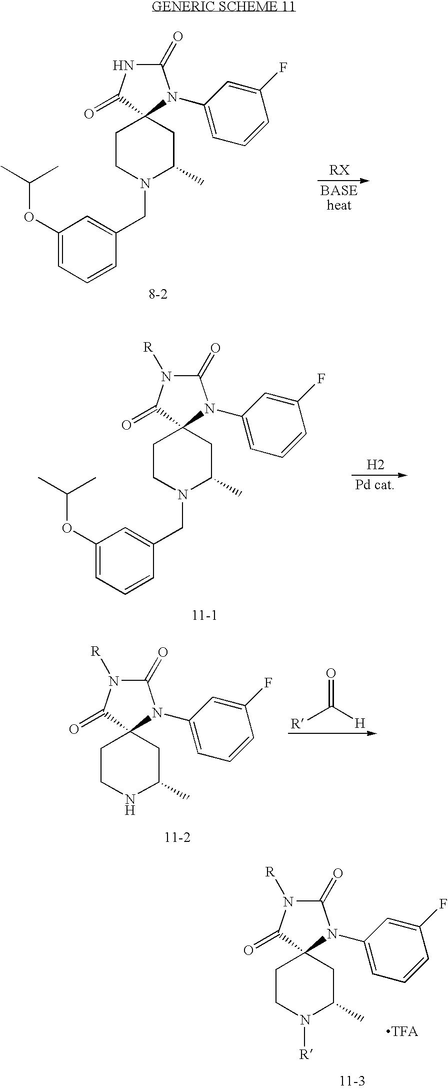

Generic Scheme 11 outlines another method of preparing compounds having different combinations of substituents on the nitrogen of the hydantoin and on the piperidine nitrogen. In this method, the hydantoin 8-2 (described in Generic Scheme 8) is treated with a suitable base like potassium carbonate and an alkylating agent to give the N-alkylated intermediate 11-1. The benzyl group on the piperidine nitrogen is then removed with hydrogenation in the presence of a palladium catalyst like palladium hydroxide to give intermediate 11-2. The piperidine nitrogen is then treated with a suitable aldehyde under reductive amination conditions with a reagent like triacetoxyborohydride to give the product 11-3.

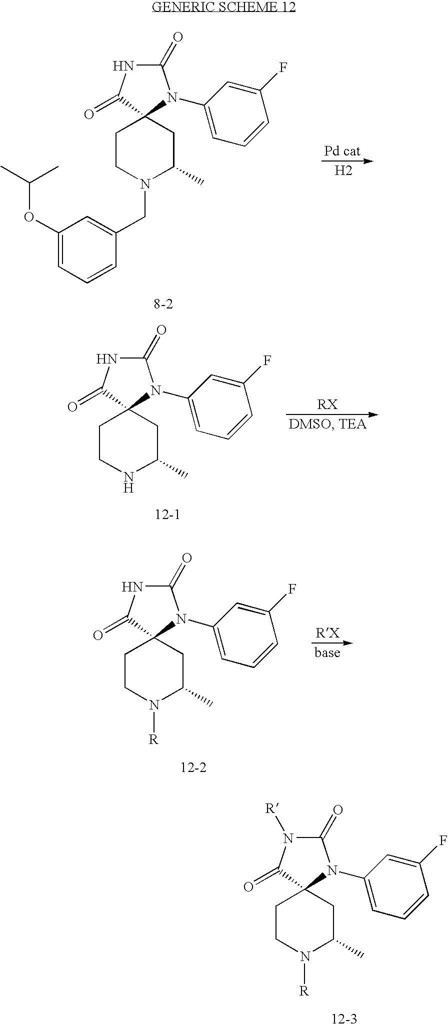

Generic Scheme 12 outlines another method of preparing compounds having different combinations of substituents on the nitrogen of the hydantoin and on the piperidine nitrogen, and allows for the piperidine substituent to be incorporated last. In this method, the benzyl group on the hydantoin 8-2 (described in Generic Scheme 8) piperidine nitrogen is removed with hydrogenation in the presence of a palladium catalyst like palladium hydroxide to give intermediate 12-1. The piperidine nitrogen is then alkylated with a suitable alkylating agent to give intermediate 12-2, which is then treated with a suitable base like potassium-carbonate and an alkylating agent to give the N-alkylated intermediate 12-3.

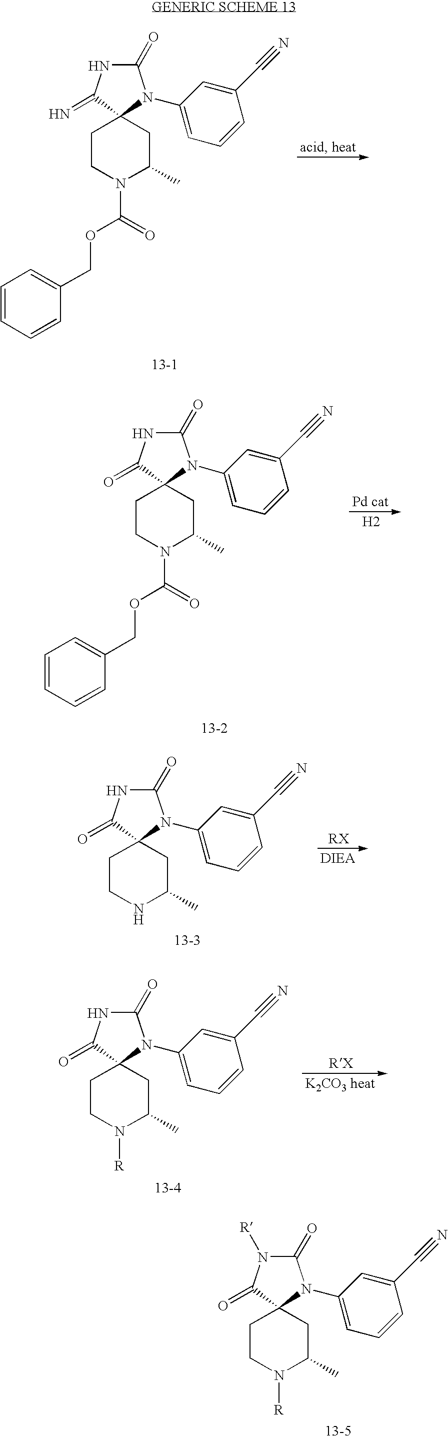

Generic Scheme 13 outlines another method of preparing compounds covered in the scope of the invention, in particular those that possess a cyano substituent on the phenyl group. Acid hydrolysis of the iminohydantoin 13-1 (prepared in a manner similar to that described for example 11-6 in WO2007/011833 by using 3-cyanoaniline and condition A) using an aqueous acid like HCl is followed by hydrogenation with a catalyst like palladium hydroxide to give the intermediate 13-3. Alkylation of the piperidine nitrogen using a base like DIEA, then alkylation of the hydantoin nitrogen using a base like K₂CO₃ gives the products 13-5.

Where they are not themselves commercially available, the starting materials and reagents described in above Generic Schemes 1-13 may be obtained from commercially available precursors by means of well known synthetic procedures and the methods disclosed in the Examples herein.

The term “substantially pure” means that the isolated material is at least 90% pure, and preferably 95% pure, and even more preferably 99% pure as assayed by analytical techniques known in the art.

The term “pharmaceutically acceptable salts” refers to salts prepared from pharmaceutically acceptable non-toxic bases or acids including inorganic or organic bases and inorganic or organic acids. The compounds of the invention may be mono, di or tris salts, depending on the number of acid functionalities present in the free base form of the compound. Free bases and salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolainine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, trifluoroacetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, trifluoroacetic, maleic, phosphoric, sulfuric, fumaric, and tartaric acids.

The present invention is directed to the use of the compounds of formulas (I) to (IV) disclosed herein as inhibitors of β-secretase enzyme activity or β-site amyloid precursor protein-cleaving enzyme (“BACE”) activity, in a patient or subject such as a mammal in need of such inhibition, comprising the administration of an effective amount of the compound. The terms “β-secretase enzyme,” “β-site amyloid precursor protein-cleaving enzyme,” and “BACE” are used interchangeably in this specification. In addition to humans, a variety of other mammals can be treated according to the method of the present invention.

The compounds of the present invention have utility in treating, ameliorating, controlling or reducing the risk of Alzheimer's disease. For example, the compounds may be useful for the prevention of dementia of the Alzheimer's type, as well as for the treatment of early stage, intermediate stage or late stage dementia of the Alzheimer's type. The compounds may also be useful in treating, ameliorating, controlling or reducing the risk of diseases mediated by abnormal cleavage of amyloid precursor protein (also referred to as APP), and other conditions that may be treated or prevented by inhibition of β-secretase. Such conditions include mild cognitive impairment, Trisomy 21 (Down Syndrome), cerebral amyloid angiopathy, degenerative dementia, Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, pancreatitis, inclusion body myositis, other peripheral amyloidoses, diabetes and atherosclerosis.

The subject or patient to whom the compounds of the present invention is administered is generally a human being, male or female, in whom inhibition of β-secretase enzyme activity is desired, but may also encompass other mammals, such as dogs, cats, mice, rats, cattle, horses, sheep, rabbits, monkeys, chimpanzees or other apes or primates, for which inhibition of β-secretase enzyme activity or treatment of the above noted disorders is desired.

The compounds of the present invention may be used in combination with one or more other drugs in the treatment of diseases or conditions for which the compounds of the present invention have utility, where the combination of the drugs together are safer or more effective than either drug alone. Additionally, the compounds of the present invention may be used in combination with one or more other drugs that treat, prevent, control, ameliorate, or reduce the risk of side effects or toxicity of the compounds of the present invention. Such other drugs may be administered, by a route and in an amount commonly used therefor, contemporaneously or sequentially with the compounds of the present invention. Accordingly, the pharmaceutical compositions of the present invention include those that contain one or more other active ingredients, in addition to the compounds of the present invention. The combinations may be administered as part of a unit dosage form combination product, or as a kit or treatment protocol wherein one or more additional drugs are administered in separate dosage forms as part of a treatment regimen.

Examples of combinations of the compounds of the present invention with other drugs in either unit dose or kit form include combinations with anti-Alzheimer's agents, for example other beta-secretase inhibitors or gamma-secretase inhibitors; glycine transport inhibitors, tau phosphorylation inhibitors; blockers of Aβ oligomer formation; p25/CDK5 inhibitors; HMG-CoA reductase inhibitors; PPAR gamma agonists, such as pioglitazone and rosiglitazone; NK1/NK3 receptor antagonists; NSAID's including ibuprofen; vitamin E; anti-amyloid antibodies, including anti-amyloid humanized monoclonal antibodies; COX-2 inhibitors; anti-inflammatory compounds, such as (R)-flurbiprofen; CB-1 receptor antagonists or CB-1 receptor inverse agonists; antibiotics such as doxycycline and rifampin; N-methyl-D-aspartate (NMDA) receptor antagonists, such as memantine and neramexane; NR2B antagonists; androgen receptor modulators; acetylcholinesterase inhibitors such as galantamine, rivastigmine, donepezil, and tacrine; mGluR5 modulators; growth hormone secretagogues such as ibutamoren, ibutamoren mesylate, and capromorelin; histamine H₃ antagonists; AMPA agonists; PDE IV inhibitors; GABA_{A} inverse agonists; GABA_{A} α 5 receptor ligands; GABA_{B} receptor ligands; potassium channel blockers; neuronal nicotinic agonists; P-450 inhibitors, such as ritonavir; or other drugs that affect receptors or enzymes that either increase the efficacy, safety, convenience, or reduce unwanted side effects or toxicity of the compounds of the present invention. The foregoing list of combinations is illustrative only and not intended to be limiting in any way.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients.

In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound, which is a compound of the invention (of formulas (I), (I′), (II), (II′), (III) and (III′)), is included in an amount sufficient to produce the desired effect upon the process or condition of diseases. Accordingly, the pharmaceutical compositions of the present invention encompass any composition made by admixing a compound of the invention and a pharmaceutically acceptable carrier.

The carrier may take a wide variety of forms depending on the form of preparation desired for administration, e.g., oral or parenteral (including intravenous). Thus, the pharmaceutical compositions of the invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, may also be administered by controlled release means and/or delivery devices.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets may contain a compound of the invention in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a composition of this invention may be prepared by compression or molding, optionally with one or more accessory ingredients or adjuvants. Compressed tablets may be prepared by compressing, in a suitable machine, a compound of the invention in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the compound of the invention.

Compositions for oral use may also be presented as hard gelatin capsules wherein the compound of the invention is mixed with an inert solid diluent, for example, calcium carbonate, calcium phosphate or kaolin, or as soft gelatin capsules wherein the compound of the invention is mixed with water or an oil medium, for example peanut oil, liquid paraffin, or olive oil.

Other pharmaceutical compositions include aqueous suspensions, which contain the active materials in admixture with excipients suitable for the manufacture of aqueous suspensions. In addition, oily suspensions may be formulated by suspending the compound of the invention in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

The pharmaceutical compositions may be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the compound of the invention, to produce a cream or ointment having a desired consistency.

Pharmaceutical compositions of this invention can also be in a form suitable for rectal administration wherein the carrier is a solid. It is preferable that the mixture forms unit dose suppositories. Suitable carriers include cocoa butter and other materials commonly used in the art.

By “pharmaceutically acceptable” it is meant the carrier, diluent or excipient must be compatible with the other ingredients of the formulation and not deleterious to the recipient thereof.

The terms “administration of or “administering a” compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” means the amount of the subject compound that will elicit the biological or medical response of a tissue, system, animal or human that is being sought by the researcher, veterinarian, medical doctor or other clinician.

As used herein, the term “treatment” or “treating” means any administration of a compound of the invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The compositions containing compounds of the invention may conveniently be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples of unit dosage forms.

The compositions containing compounds of the invention may conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

When treating, ameliorating, controlling or reducing the risk of Alzheimer's disease or other diseases for which compounds of the invention are indicated, generally satisfactory results are obtained when the compounds of the invention are administered at a daily dosage of from about 0.1 mg to about 100 mg per kg of animal body weight, preferably given as a single daily dose or in divided doses two to six times a day, or in sustained release form. The total daily dosage is from about 1.0 mg to about 2000 mg, preferably from about 0.1 mg to about 20 mg per kg of body weight. In the case of a 70 kg adult human, the total daily dose will generally be from about 7 mg to about 1,400 mg. This dosage regimen may be adjusted to provide the optimal therapeutic response. The compounds may be administered on a regimen of 1 to 4 times per day, preferably once or twice per day.

The amount of the compound of the invention that may be combined with the carrier materials to produce a single dosage form will vary depending upon the host treated and the particular mode of administration. For example, a formulation intended for the oral administration to humans may conveniently contain from about 0.005 mg to about 2.5 g of a compound of the invention, compounded with an appropriate and convenient amount of carrier material. Unit dosage forms will generally contain between from about 0.005 mg to about 1000 mg of the compound of the invention, typically 0.005 mg, 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg, administered once, twice or three times a day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The utility of the compounds in accordance with the present invention as inhibitors of β-secretase enzyme activity may be demonstrated by methodology known in the art. Enzyme inhibition is determined as follows.

ECL Assay: A homogeneous end point electrochemiluminescence (ECL) assay is performed using a biotinylated BACE substrate. The Km of the substrate is greater than 100 μM and can not be determined due to the limit of solubility of the substrate. A typical reaction contains approximately 0.1 nM enzyme, 0.25 μM of the substrate, and buffer (50 mM NaOAc, pH 4.5, 0.1 mg/ml BSA, 0.2% CHAPS, 15 mM EDTA and 1 mM deferoxamine) in a total reaction volume of 100 μl. The reaction proceeds for 30 min and is then stopped by the addition of 25 μL of 1 M Tris-HCl, pH 8.0. The resulting enzymatic product is assayed by adding a ruthenylated antibody which specifically recognizes the C-terminal residue of the product. Streptavidin coated magnetic beads are added into the solution and the samples are subjected to M-384 (Igen Inc., Gaithersburg, Md.) analysis. Under these conditions, less than 10% of substrate is processed by BACE 1. The enzyme used in these studies is soluble (transmembrane domain and cytoplasmic extension excluded) human protein produced in a baculovirus expression system. To measure the inhibitory potency for compounds, 12 concentrations of inhibitors are prepared starting from 100 μM with three fold series dilution. Solutions of the inhibitor in DMSO are included in the reaction mixture (final DMSO concentration is 10%). All experiments are conducted at rt using the standard reaction conditions described above. To determine the IC₅₀ of the compound, a four parameter equation is used for curve fitting. The errors in reproducing the dissociation constants are typically less than two-fold.

In particular, the compounds of the following examples had activity in inhibiting the beta-secretase enzyme in the aforementioned assay, generally with an IC₅₀ from about 1 nM to 200 μM. Such a result is indicative of the intrinsic activity of the compounds in use as inhibitors of beta-secretase enzyme activity.

Several methods for preparing the compounds of this invention are illustrated in the Schemes and Examples herein. Starting materials are made according to procedures known in the art or as illustrated herein. The following examples are provided so that the invention might be more fully understood. These examples are illustrative only and should not be construed as limiting the invention in any way.

### Intermediates

### Intermediate I

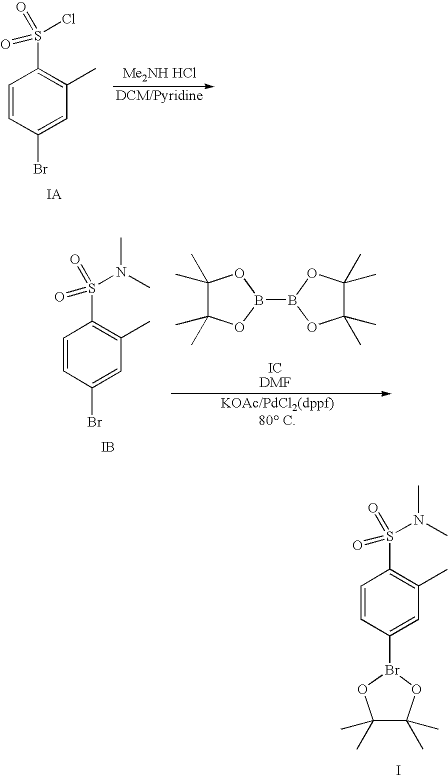

Step 1: IA (1.03 g, 0.00383 mmol) was added to a stirred solution of dimethylamine hydrochloric acid (0.620 g, 2.0 eq) and pyridine (1.55 ml, 5.0 eq) in DCM (15 ml) at 0° C. After stirring for a while, the reaction mixture was allowed to warm up to rt and stirred overnight. The mixture was extracted with Et₂O/saturated NaHCO₃ aqueous solution twice, and Et₂O/NaH₂PO₄ aqueous solution (pH=3-4) twice, washed with brine twice, dried with MgSO₄ and concentrated to afford IB.

EI-MS m/z: 278, 280 (M+H)⁺

Step 2: IB (0.253 g, 0.910 mol), KOAc (0.268 g, 3.0 eq), ID (0.208 g, 0.9 eq) and PdCl₂ (dppf) (0.074 g, 0.1 eq) were added in a round bottom flask. Dry DMF (5 ml) was then added under N₂ protection. The resultant mixture was stirred for a while and put into an 80° C. oil bath. The reaction was followed by LCMS. Upon completion (2-4 h), reaction mixture was extracted with EtOAc/water three or four times, washed with brine twice. The resultant EtOAc solution was dried with MgSO₄ and concentrated to afford crude Intermediate I.

EI-MS m/z: 326 (M+H)⁺

### Intermediate II

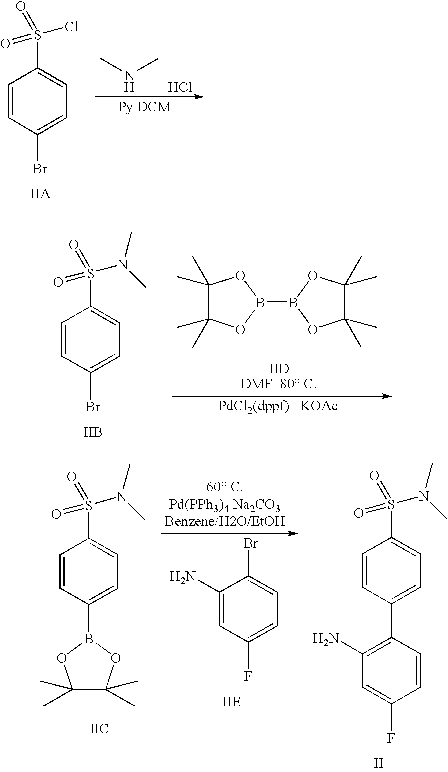

Step 1: IIA (4.874 g, 0.0191 mmol) was added to a stirred solution of Dimethylamine hydrochloric acid (2.330 g, 1.5 eq) and pyridine (8.0 ml, 5.2 eq) in DCM (25 ml) at 0° C. After stirring for a while, the reaction mixture was allowed to warm up to rt and the reaction was followed by LCMS. Upon completion (about 3 h), the mixture was extracted with Et₂O/saturated NaHCO₃ aqueous solution twice, and Et₂O/NaH₂PO₄ aqueous solution (pH=3-4) twice, washed with brine twice, dried with MgSO₄ and concentrated to afford IIB.

EI-MS m/z: 266, 264 (M+H)⁺

Step 2: B (3.21 g, 0.0122 mol), KOAc (3.58 g, 3.0 eq), IID (4.01 g, 1.3 eq) and PdCl₂ (dppf) (0.993 g, 0.1 eq) were added in a round bottom flask. Dry DMF (60 ml) was then added under N₂ protection. The resultant mixture was stirred for a while and put into an 80° C. oil bath. The reaction was followed by LCMS. Upon completion (2-4 hrs), reaction mixture was extracted with EtOAc/water three or four times, washed with brine twice. The resultant EtOAc solution was dried with MgSO₄ and concentrated to afford crude IIC. EI-MS m/z: 312 (M+H)⁺.

Crude IIC (4.80 g, 0.0122 mol), IIE (3.01 g, 1.3 eq), Pd(PPh₃)₄(1.41 g, 0.1 eq), Na₂CO₃ (7.76 g, 6.0eq), benzene (32 ml), H₂O (16 ml) and EtOH (4 ml) were mixed. The mixture was stirred and heated at 60° C. overnight. Once the mixture cooled down, in was mixture filtered. The solid was washed with water and Et₂O. The organic phase was concentrated, redissolved in benzene with heating, precipitated with addition of hexane, washed with DCM and combine two batches of solid to afford Intermediate II. EI-MS m/z: 295 (M+H)⁺

### Intermediate III

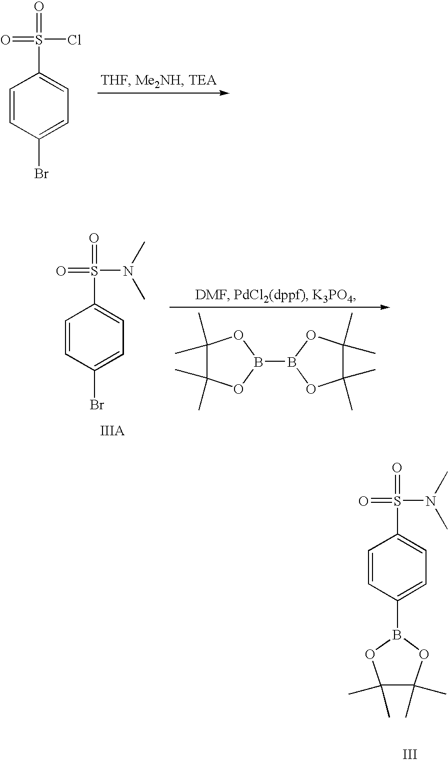

Step 1: To a stirred solution of 4-bromobenzenesulfonyl chloride (2.5 g, 10.0 mmol) in 50.0 mL THF at 0° C., was added 10.0 mL of 2M dimethylamine in THF. The reaction was stirred at it for 3 h, then quenched with water. The mixture was extracted three times with ethyl acetate, washed with water, 1N HCl and brine. The solution was dried over magnesium sulfate, filtered, and concentrated to yield IIIA as a white solid which was used for next reaction step without purification.

EI-MS m/z: 264.00 (M+H)⁺.

Step 2: A mixture of IIIA (2.4 g, 9.0 mmol), bis(pinacolato)diboron (2.5 g, 10.0 mmol) and potassium phosphate (42.5 g, 20.0 mmol) in 50 mL DMF, was purged with nitrogen gas, then added PdCl₂(dppf) (4.0 g, 5.6 mmol). The solution was heated at 80° C. for 3 h, cooled to rt and quenched with water. The product was extracted three times with ethyl acetate, washed with water, saturated sodium bicarbonate and brine. The solution was dried over magnesium sulfate, filtered, concentrated then purified by silica gel column chromatography (EtOAc-Hex 1:1) to yield Intermediate III as an off-white solid.

EI-MS m/z: 312.15 (M+H)⁺.

Intermediate IV

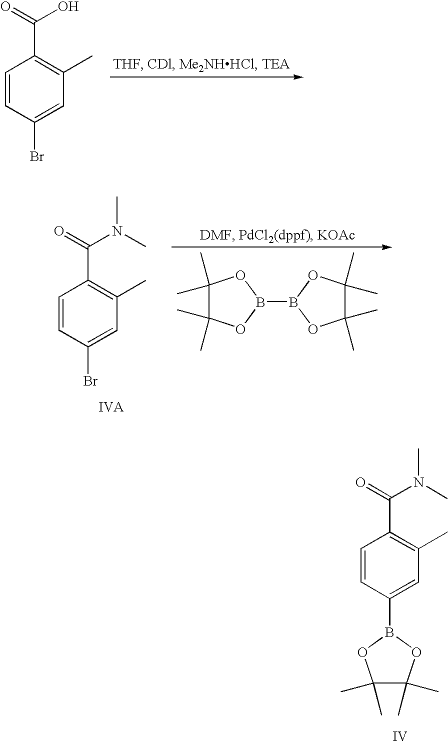

Step 1: To a stirred solution of 4-bromo-2-methylbenzoic acid (0.43 g, 2.0 mmol) in 10.0 mL THF, was added carbonyl diimidazole (0.356 g, 2.2 mmol). The resulting mixture was stirred at rt for 5 min, then added dimethylamine hydrochloride salt (0.179 g, 2.2 mmol), followed by triethylamine (0.56 mL, 4.0 mmol). The solution was stirred at rt for 3 h, then quenched with water. The product was extracted three times with ethyl acetate, washed with water, saturated sodium bicarbonate, 1N HCl and brine. The solution was dried over magnesium sulfate, filtered, and concentrated to yield IVA as a white solid which was used for next step without purification.

EI-MS m/z: 244.0.5 (M+H)⁺.

Step 2: A mixture of IVA (0.376 g, 1.55 mmol), bis-(pinacolato)diboron (0.472 g, 1.86 mmol) and potassium acetate (0.761 g, 7.75 mmol) in 6.0 mL DMF, was purged with nitrogen gas, then added PdCl₂(dppf) (340.0 mg, 0.465 mmol). The solution was heated at 80° C. for 3 h, cooled to rt, then quenched with water. The product was extracted three times with ethyl acetate, washed with water, saturated sodium bicarbonate, brine, and dried over magnesium sulfate, filtered, concentrated and purified by silica gel column chromatography (EtOAc-Hex 1:1) to yield Intermediate IV as an off-white solid.

EI-MS m/z: 290.25 (M+H)⁺.

### Intermediate V

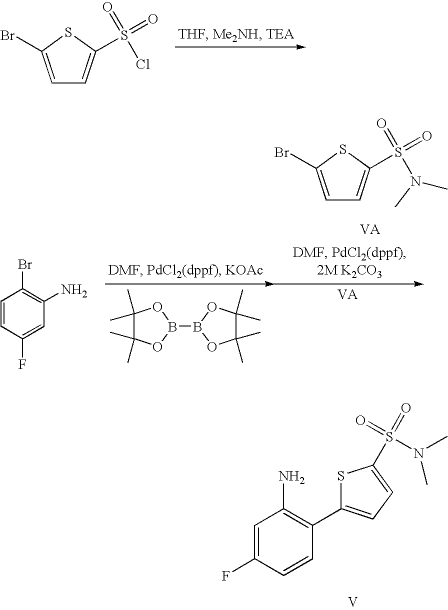

Step 1: To a stirred solution of 5-bromo-thiophene-2-sulfonyl chloride (1.05 g, 4.0 mmol) in 20.0 mL THF at 0° C., was added 4.0 mL of 2M dimethylamine in THF. The solution was stirred at rt for 3 h, then quenched with water. The product was extracted three times with ethyl acetate, washed with water, 1N HCl and brine. The solution was dried over magnesium sulfate, filtered, and concentrated to yield VA as a solid which was used for next reaction step without purification.

EI-MS m/z: 271.95 (M+H)⁺.

Step 2: A mixture of 2-bromo-5-fluoro-phenylamine (380.0 mg, 2.0 mmol), bis(pinacolato)-diboron (559.0 mg, 2.2 mmol) and potassium acetate (981.0 mg, 10.0 mmol) in 10.0 mL DMF, was purged with nitrogen gas, then added PdCl₂(dppf) (292.4.0 mg, 0.4 mmol). The solution was heated at 80° C. for 3 h, cooled to rt: To the resulting reaction mixture was added 5.0 mL DMF, compound VA (810.0 mg, (3.0 mmol) and 5.0 mL of 2M K₂CO₃, purged with nitrogen gas, then added PdCl₂(dppf) (292.4.0 mg, 0.4 mmol). The reaction mixture was heated at 80° C. for 3 h, cooled to rt, and quenched with water. The product was extracted three times with ethyl acetate, washed with water, saturated sodium bicarbonate, brine, and dried over magnesium sulfate. The solution was filtered, concentrated and purified by silica gel column chromatography (EtOAc-Hex 3:7) to yield Intermediate V as an oily residue.

EI-MS m/z: 301.05 (M+H)⁺.

### Intermediate VI

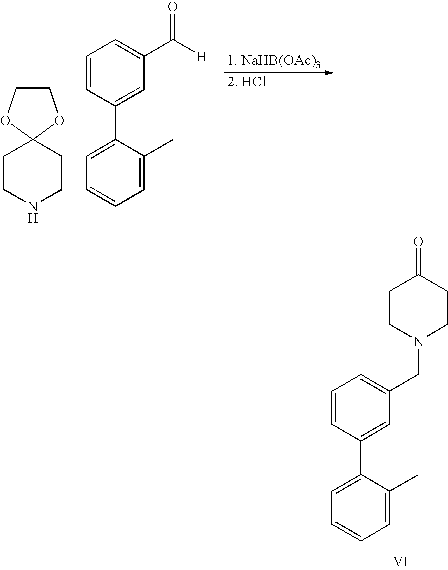

Step 1: 1-[(2′-methylbiphenyl-3-yl)methyl]piperidin-4-one (Intermediate VI) To a suspension of 1,4-dioxa-8-azaspiro[4.5]decane (3.5 ml, 27.0 mmol) in 1,2-dichloroethane (200 ml) were added 2′-methylbiphenyl-3-carbaldehyde (5.0 g, 25.5 mmol) and acetic acid (1 ml). The reaction mixture was allowed to stir for 1 h at rt. Sodium triacetoxyborohydride (10.8 g, 51.0 mmol) was added to the reaction, and the mixture continued to stir for 18 h at it. The reaction was quenched by the addition of a saturated sodium bicarbonate solution, diluted with dichloromethane, and allowed to stir vigorously for 1 h. The organic portion was separated, washed with brine, dried over sodium sulfate, filtered, and concentrated under vacuum. The crude oil was purified via flash chromatography (silica gel, 5% methanol/DCM) to yield 842% methylbiphenyl-3-yl)methyl]-1,4-dioxa-8-azaspiro[4.5]decane (54%). The material was dissolved in concentrated HCl (5.0 ml), stirred at it for 12 h, and then at reflux for 2 days. The reaction was concentrated under vacuum and quenched with saturated sodium bicarbonate solution. The product was extracted with dichloromethane, and the organic portion was dried over sodium sulfate, filtered, and concentrated under vacuum. The material was purified via flash chromatography (silica gel, 0-5% methanol/dichloromethane) to yield 14(2′-methylbiphenyl-3-yl)methyl]piperidin-4-one.

¹H NMR (400 MHz, CDCl₃) δ 7.39 (m, 1H), 7.33 (m, 2H), 7.26 (m, 5H), 3.68 (s, 2H), 2.78 (t, J=6.0 Hz, 4H), 2.46 (t, J=6.2 Hz, 4H), 2.28 (s, 3H).

LCMS (M+H) 280.2.

### Example 1

### 1-(3-fluorophenyl)-8-[(2′-methyl-1,1′-biphenyl-3-yl)methyl]-2,4-dioxo-1,3-diaza-8-azoniaspiro[4.5]decane trifluoroacetate

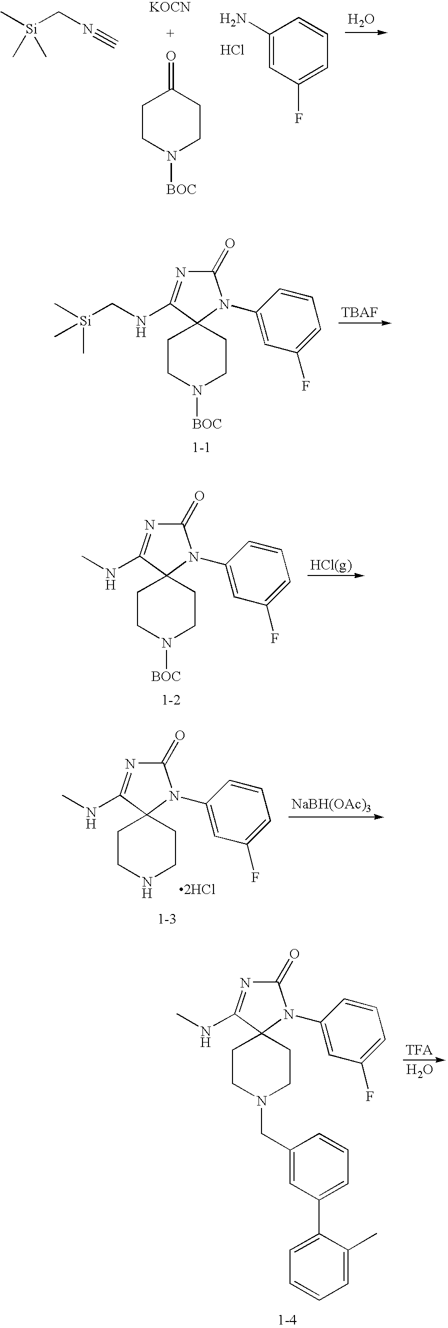

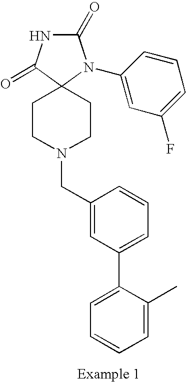

### Step 1; tert-Butyl 1-(3-fluorophenyl)-2-oxo-4-{[(trimethylsilyl)methyl]amino}-1,3,8-triazaspiro[4.5]dec-3-ene-8-carboxylate 1-1

To a 4 mL MeOH suspension of 2.0 g (10.0 mmol) N-boc piperidinone and 1.35 ml (9.5 mmol) trimethylsilyl methylisocyanide was added a solution of 1.02 g (12.5 mmol) potassium isocyanate in 2.1 mL H₂O in one portion with stirring followed by 1.48 g (10.0 mmol) 3-fluoroaniline hydrochloride in portions over 5 min. After stirring for 2 h the reaction was treated with 250 ml CH₂Cl₂. The organic layer was washed with water (2×50 ml), brine (1×50 ml), dried over Na₂SO₄, filtered and concentrated to dryness under vacuum to give a crude oil. Purification by automated flash chromatography (0-5.5% MeOH in CH₂Cl₂ over 28 min) afforded 1.04 g tert-butyl 1-(3-fluorophenyl)-2-oxo-4-{[(trimethylsilyl)methyl]amino}-1,3,8-triazaspiro[4.5]dec-3-ene-8-carboxylate as a white solid.

Electrospray mass spectrum: M+H=449.2

### Step 2: tert-Butyl 1-(3-fluorophenyl)-4-(methylamino)-2-oxo-1,3,8-triazaspiro[4.5]dec-3-ene-8-carboxylate 1-2

To a 50 ml THF solution of 1.04 g (2.41 mmol) tert-butyl 1-(3-fluorophenyl)-2-oxo-4-{[(trimethylsilyl)methyl]amino}-1,3,8-triazaspiro[4.5]dec-3-ene-8-carboxylate was added 3.61 mL (3.61 mmol) of a 1.0M tetrabutylammonium fluoride in THF solution over 5 min and the reaction warmed to 60° C. overnight. The reaction was concentrated to dryness under vacuum and the residue treated with 100 mL CH₂Cl₂. The organic layer was washed with water (1×25 mL), brine (1×25 mL), dried over Na2SO4, filtered and concentrated to dryness under vacuum to give a crude oil. ‘Purification by automated flash chromatography (0-7.5% MeOH in CH₂Cl₂ over 25 min) afforded 0.854 g tert-butyl 1-(3-fluorophenyl)-4-(methylamino)-2-oxo-1,3,8-triazaspiro[4.5]dec-3-ene-8-carboxylate as a white solid. High resolution mass spec (FT/ICR): calc M+H=377.1984, found 377.2010

### Step 3: 1-(3-fluorophenyl)-4-methylammonio)-2-oxo-1,3-diaza-8-azoniaspiro[4.5]dec-3-ene Dihydrochloride 1-3

To a suspension of 834 mg (2.21 mmol) tert-butyl 1-(3-fluorophenyl)-4-(methylamino)-2-oxo-1,3,8-triazaspiro[4.5]dec-3-ene-8-carboxylate in 10mL EtOAc at 0° C. was bubbled in HCl gas until the solvent was saturated. The reaction was stirred in the cold for 30 m and concentrated under vacuum. The solid residue was reconcentrated to dryness (2×ethyl ether) and dried under high vacuum to give 1-(3-fluorophenyl)-4-methylamino)-1,3,8-triazaspiro[4.5]dec-3-ene-2-one as its dihydrochloride salt as a fine white solid. High resolution mass spec (FT/ICR): calc M+H (free base)=277.1459, found 277.1465

### Step 4: 1-(3-fluorophenyl)-8-[(2′-methyl-1,1′-biphenyl-3-yl)methyl]-2,4-dioxo-1,3-diaza-8-azoniaspiro[4.5]decane trifluoroacetate (Example 1)

To a suspension of 35 mg (0.100 mmol) 1-(3-fluorophenyl)-4-methylammonio)-2-oxo-1,3-diaza-8-azoniaspiro[4.5]dec-3-ene dichloride, 26 uL (0.150 mmol) diisopropylethylamine, and 30 mg (0.140 mmol) triacetoxy sodium borohydride in 1.0 mL of DCE was added 20 mg 2′-methyl-(1,1′-biphenyl)-3-carboxaldehyde (Oakwood Products Inc.). The resulting heterogeneous mixture was stirred at rt under a nitrogen atmosphere for 2 h. The reaction mixture was treated with 20 uL saturated NaHCO₃ (aq) and concentrated under vacuum. Purification of the residue by preparative HPLC (5→95% CH₃CN/H₂O over 30 min, 0.05% added TFA, C18 PRO YMC 20×150 mm) afforded 1-(3-fluorophenyl)-4-(methylamino)-8-[(2′-methyl-1,1′-biphenyl-3-yl)methyl]-1,3,8-triazaspiro[4.5]dec-3-en-2-one (1-4, electrospray mass spectrum: M+H=457.2) as a solution in aqueous acetonitrile containing 0.05% trifluoroacetic acid which was converted to 1-(3-fluorophenyl)-8-[(2′-methyl-1,1′-biphenyl-3-yl)methyl]-1,3,8-triazaspiro[4.5]decane-2,4-dione by heating this solution at 60° C. for 48 h. Lyophilization afforded a white solid as the monofluoroacetic acid salt of the title compound.

¹H NMR (CD₃OD with 1-4 mg K₂CO_{3 (s)}, 400 MHz): δ 7.48 (m, 1H), 7.35 (m, 1H), 7.21 (m, 7H), 7.10 (m, 3H), 3.58 (s, 2H), 2.86 (m, 2H), 2.75 (m, 2H), 2.19 (s, 3H), 2.02 (m, 2H), 1.91 (m, 2H). High resolution mass spec (FT/ICR): calc M+H (free base)=444.2082, found 444.2125

### Example 2

### 4′-fluoro -2′-[8-(3-isopropoxybenzyl)-2,4-dioxo-1,3,8-triazaspiro[4.5]dec-1-yl]-n,n,3-trimethylbiphenyl-4-sulfonamide

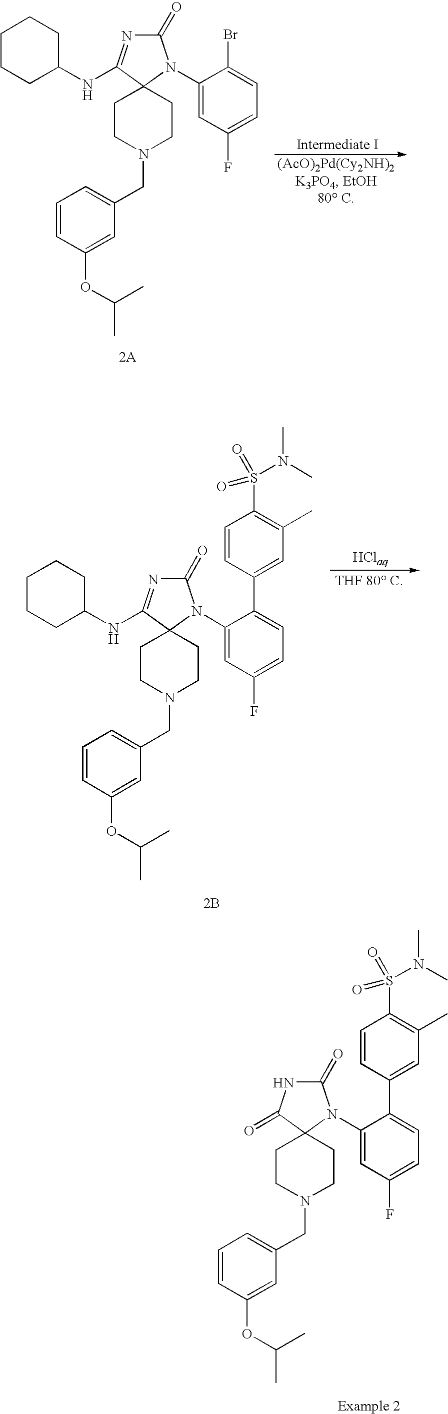

Step 1: Compound 2A (0.0950 g, 0.166 mmol, compound 15-1 in International Patent Publication WO2006/044497) and Intermediate I (0.131 g, 2.0 eq), the palladium catalyst (AcO)₂Pd(Cy₂NH)₂ (0.0200 g, 0.65 eq) and K₃PO₄ (0.0340 g, 3.0 eq) were weighed into a 1 dram vial. A magnetic stir bar was added, followed by absolute ethanol (1 mL). The vial was capped, and placed to stir in a 80 deg C aluminum block over a hot plate. When the reaction showed black palladium precipitate (several hours), the reaction was cooled to room temperature. The reaction was then filtered through celite, and the resulting solution was purified by HPLC to afford Compound 2B. EI-MS m/z: 690 (M+H)⁺

Compound 2B (0.020 g, 0.0290 mmol), 1M aqueous HCl (5m1), and THF (1 ml) were added. The resultant mixture was stirred and heated at 80° C. overnight. The mixture was extracted with EtOAc/saturated NaHCO₃ aqueous solution twice, washed with brine twice, dried with MgSO₄, concentrated and purified by HPLC to afford Example 2.

EI-MS m/z: 609 (M+H)⁺.

### Examples 3-5

### 1-{4′-[(dimethylamino)sulfonyl]-4-fluorobiphenyl-2-yl}-8-(3 -isopropoxybenzyl)-2,4-dioxo-1,3-diaza-8-azoniaspiro[4.5]decane trifluoroacetate (Example 3)

### 4′-fluoro-2′-[8-(3-isopropoxybenzyl)-3-methyl-2,4-dioxo-1,3,8-triazaspiro[4.5]dec-1-yl]-N,N-dimethylbiphenyl-4- sulfonamide (Example 4)

### 4′-fluoro-2′-[8-(3-isopropoxybenzyl)-2,4-dioxo-3-propyl-1,3,8-triazaspiro[4.5]dec-1-yl]-N,N-dimethythiphenyl-4-sulfonamide (Example 5)

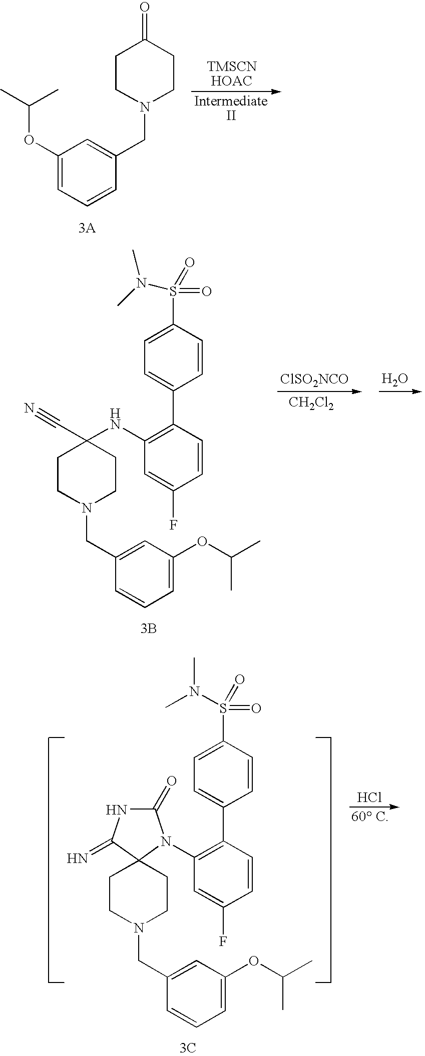

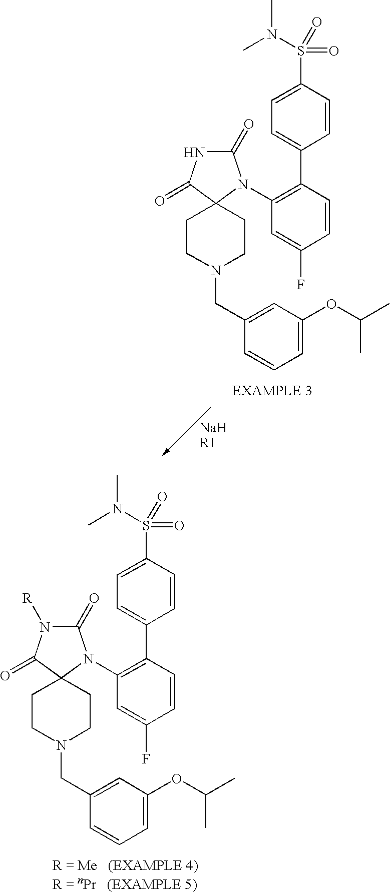

Step 3: Intermediate II (2.80 g, 0.00951 mol), Compound 3A (Example 14-4 of International Patent Publication WO2006/044497) 2.77 g, 1.1 eq), HOAc (78 ml) were mixed and heated at 60° C. until the solid was dissolved. The reaction mixture was cooled down. Trimethylsilyl cyanide (3.80 ml, 3.0 eq) added dropwise at 0° C. The reaction mixture was then allowed to warm up to rt and stirred overnight. The reaction was quenched with ammonium hydroxide in ice until the pH reached 10, then filtered. The solid was washed with water and Et₂O and dried under vacuum at 40° C. to afford compound 3B. EI-MS m/z: 295 (M+H)⁺.

Step 4: To the solution of Compound 3B (0.250 g, 0.454 mmol) in DCM (3 ml), was added dropwise at 0° C. chlorosulfonyl isocyanate (0.043 ml, 1.1 eq). The mixture was stirred at rt for 35 min. Water (1 ml) was then added. The reaction mixture was stirred for 1 h. 1M aqueous HCl (9 ml) and THF (1-2 ml) were added and the resulting mixture was stirred and heated at 60° C. for 2 days. The mixture was extracted with EtOAc/saturated NaHCO₃ aqueous solution twice, washed with brine twice, dried with MgSO₄, concentrated and purified by HPLC to afford Example 3.

EI-MS m/z: 595 (M+H)⁺

Step 5: To a stirred solution of Example 3 (0.020 g, 0.0336 mmol) in DMF (1.5 ml), was added NaH (60% dispersion in mineral oil) (0.013 g, 10 eq) at 0° C. MeI (0.0020 ml, 1.0 eq) was then added. The reaction was allowed to warm up to rt and stirred for another 30 min, then quenched with saturated aqueous NH₄Cl solution, extracted with Et₂O twice, washed with brine twice, dried with MgSO₄, concentrated and purified by preparative TLC (0.8% 2M ammonia in MeOH/DCM) to afford Example 4.

EI-MS m/z: 609 (M+H)⁺

Step 6: To a stirred solution of Example 3 (0.020 g, 0.0336 mmol) in DMF (1.5 ml), was added NaH (60% dispersion in mineral oil) (0.013 g, 10 eq) at 0° C. ⁿPrI (0.0048 ml, 1.0 eq) was then added. The reaction mixture was warmed to rt and stirred for another 30 min. The reaction was quenched with saturated aqueous NH₄Cl solution, extracted with Et₂O twice, washed with brine twice, dried with MgSO₄, concentrated and purified by preparative TLC (0.5% 2M ammonia in MeOH/DCM) to give Example 5.

EI-MS m/z: 637 (M+H)⁺.

### Example 6

### 1-{4′-[(dimethylamino)sulfonyl]-4-fluorobiphenyl-2-yl}-8-(3-isopropoxybenzyl)-2,4-dioxo-1,3-diaza-8-azoniaspiro[4.5]decane trifluoroacetate (Alternative preparation of Example 3).

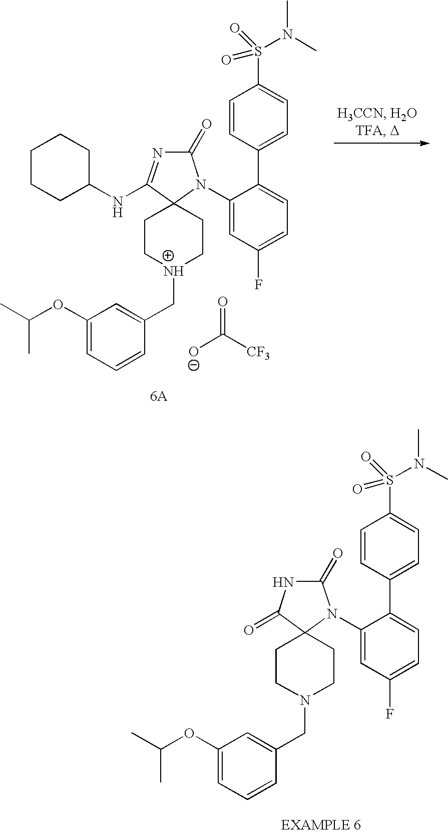

4-(cyclohexylamino)-1-{4′-[(dimethylamino)sulfonyl]-4-fluorobiphenyl-2-yl}-8-(3-isopropoxybenzyl)-2-oxo-1,3-diaza-8-azoniaspiro[4.5]dec-3-ene trifluoroacetate (6A TFA salt, prepared as previously described in International Patent Publication WO2006/044497, Example 15-2), 96.0 mg, 0.122 mmol, 1.0 equiv) was dissolved into a mixture acetonitrile (2 mL) and water (1 mL) in a vial. TFA (0.10 mL) was added, and the reaction was heated in an aluminum block at 90° C. for 3 h, until LCMS indicated disappearance of the starting material (M+H)⁺ ion. The reaction was then cooled to rt, and submitted directly for purification by reverse phase HPLC in acetonitrile/water, providing the desired spirocyclic Example 6 (27.9 mg, 32%) as its TFA salt.

El-MS m/z: 595 (M+H)⁺.

### Example 7

### 8-benzyl-1-{4′-[(dimethylamino)sulfonyl]-4-fluorobiphenyl-2-yl}-2,4-dioxo-1,3-diaza-8-azoniaspiro[4.5]decane trifluoroacetate

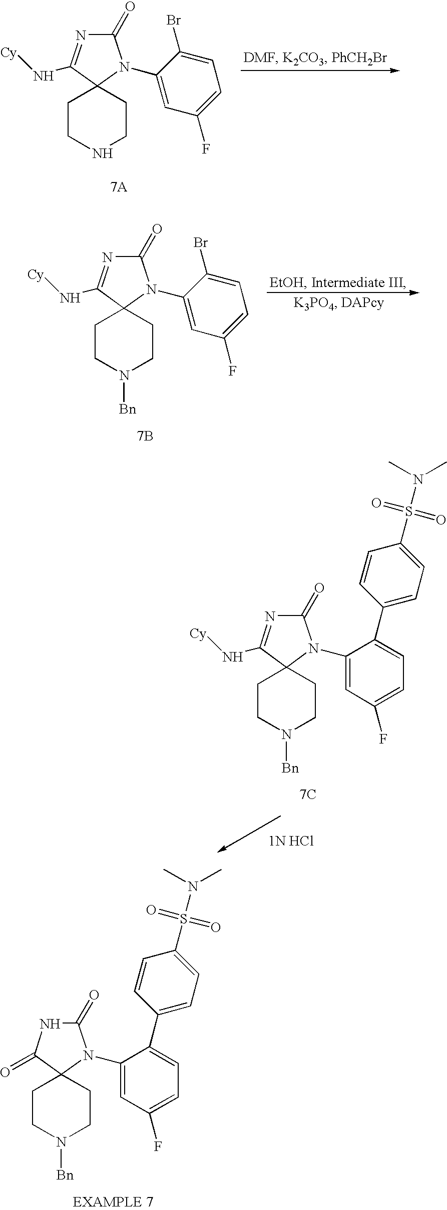

Step 3: To a solution of the amine 7A (Example 15-46 in International Patent Publication WO2006/044497) (84.7 mg, 0.2 mmol) in 2.0 mL DMF, was added potassium carbonate (30.4 mg, 0.22 mmol), followed by benzyl bromide (27.0 μL, 0.22 mmol). The solution was heated at 80° C. for 15 h, cooled to rt then quenched with water. The product was extracted three times with ethyl acetate, washed with water and brine. The solution was dried over magnesium sulfate, filtered, concentrated and purified by silica gel column chromatography (100% EtOAc) to provide 7B.

EI-MS m/z: 513.15 (M+H)⁺.

Step 4: A mixture of 7B (50.2 mg, 0.1 mmol), Intermediate III (62.2 mg, 0.2 mmol), potassium phosphate (43.6 mg, 0.2 mmol), and in 2.0 mL ethanol, was purged with nitrogen gas, then added DAPCy (58.6 mg, 0.1 mmol). The solution was heated at 80° C. for overnight, or until complete disappearance of compound 7B (monitored by LCMS). The crude was purified by preparative HPLC to provide 7C as an amine-TFA salt.

El-MS m/z: 618.30 (M+H)⁺.

Step 5: A suspension of compound 7C (10.0 mg, 0.014 mmol) in 1.0 mL of 1M HCl was heated at 80° C. overnight. The final product was purified by preparative HPLC to provide Example 7 as an amine-TFA salt.

¹H NMR (CD₃OD): δ ppm 0.94 (d, J=14.67 Hz, 1H), 1.45 (m, 1H,) 1.94 (m, 1H), 2.38 (d, J=14.67 Hz, 1H), 2.69 (s, 6H), 3.14 (d, J=13.21 Hz, 1H), 3.51 (m, 3H) 4.24 (s, 2H), 7.43 (m, 6H), 7.65 (d, J=7.83 Hz, 3H), 7.83 (d, J=8.31 Hz, 2H).

EI-MS m/z: 537.15 (M+H)⁺.

### Example 8

### 1-{4′-[(dimethylamino)carbonyl]-4-fluoro-3′-methylbiphenyl-2-yl}-8-(3-isopropoxybenzyl)-2.4-dioxo-1,3-diaza-8-azoniaspiro[4.5]decane trifluoroacetate

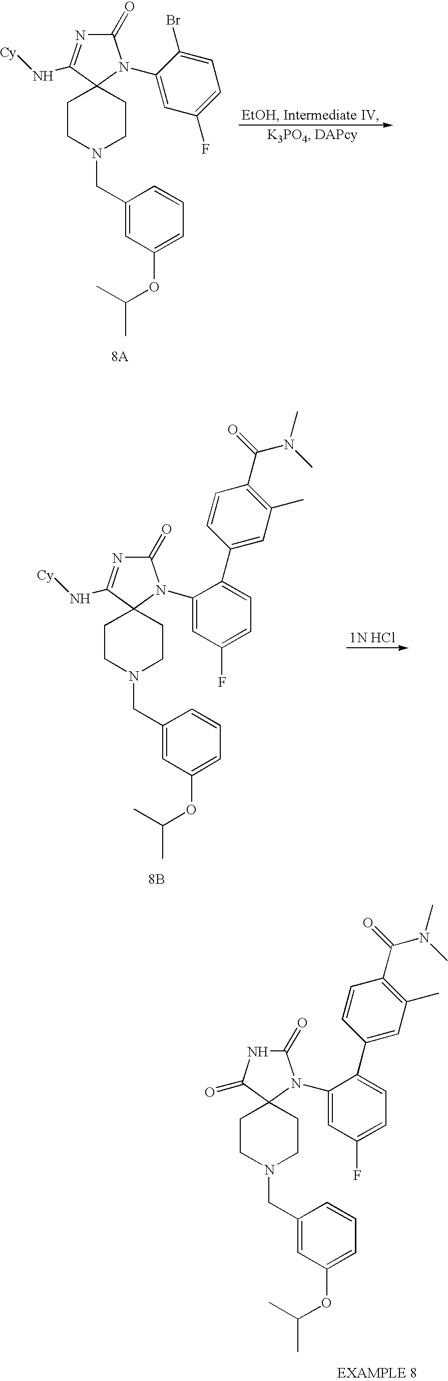

Step 1: A mixture of 8A (compound 2A from Example 2) (60.0 mg, 0.1 mmol), Intermediate IV (31.9 mg, 0.11 mmol), potassium phosphate (42.5 mg, 0.2 mmol), and in 2.0 mL ethanol, was purged with nitrogen gas, then added DAPCy (29.0 mg, 0.05 mmol). The solution was heated at 80° C. until complete disappearance of compound 8A (monitored by LCMS). The crude reaction mixture was purified by preparative HPLC to provide 8B as the amine-TFA salt.

EI-MS m/z: 654.30 (M+H)⁺.

Step 4: A suspension of compound 8B (10.0 mg, 0.013 mmol) in 1.0 mL of 1M HCl was heated at 80° C. overnight. The final product was purified by preparative HPLC to provide Example 8 as an amine-TFA salt.

¹H NMR (CD₃OD): δ ppm 1.01 (d, J=14.67 Hz, 1H), 1.29 (s, 6H), 1.45 (m, 14.18 Hz, 1H), 1.97 (t, J=13.94 Hz, 1H), 2.26 (s, 3H), 2.36 (d, J=15.65 Hz, 1H), 2.88 (s, 3H), 3.13 (s, 4H), 3.47 (m, 2H), 3.60 (t, J=12.72 Hz, 1H), 4.20 (s, 2H), 4.60 (s, 1H), 6.97 (m, 3H), 7.32 (m, 6H), 7.60 (s, 1H).

EI-MS m/z: 573.3 (M+H)⁺.

### Example 9

### 1-(2-{5-[(dimethylamino)sulfonyl]-2-thienyl}-5-fluorophenyl)-8-(3-isopropoxybenzyl)-2,4-dioxo-1,3-diaza-8-azoniaspiro[4.5]decane trifluoroacetate

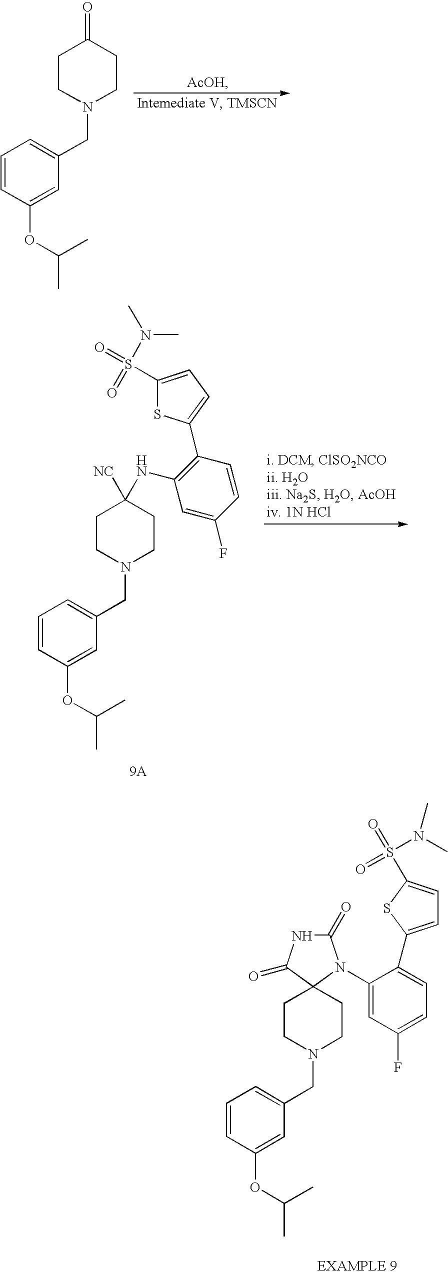

Step 1: To an ice-cold solution of 1-(3-isopropoxybenzyl)-piperidin-4-one (Example 14-4 of International Patent Publication WO2006/044497) 79.5 mg, 0.3 mmol) and Intermediate V (99.4 mg, 0.33 mmol) in glacial acetic acid (1.5 mL), was added slowly trimethylsilyl cyanide (44.0 μL, 0.33 mmol). The reaction mixture was kept at 0° C. for 5 min then rt for 30 min. The resulting mixture was quenched with ammonium hydroxide in ice until the pH reached 10 then extracted two times with dichloromethane. The combined dichloromethane extract was washed with brine, dried over magnesium sulfate and concentrated. The product was purified by silica gel column (40% ethylacetate/60% hexane) to provide 9A as an oil residue.

EI-MS m/z: 557.25 (M+H)⁺.

Step 4: To an ice cold solution of 9A (108.4 mg, 0.19 mmol) in 1.0 mL chloroform, was added dropwise chlorosulfonyl isocyanate (49.7 μL, 0.22 mmol) and stirred at rt for 30 min, then added 0.50 mL of water. The reaction mixture was stirred for another 1 h at rt then added to an ice cold aqueous solution of H₂S (156.1 mg Na₂S+4.0 mL H₂O+1.0 mL acetic acid), which was prepared right before use. The resulting reaction was stirred at rt for 24 h then hydrolyzed with 1.0 mL 1N HCl at 80° C. over a 5 h period. The reaction mixture was made basic with saturated sodium bicarbonate solution, and extracted twice with ethyl acetate. The combined extracts were washed with brine, dried over magnesium sulfate and concentrated. The final product was purified by preparative HPLC to provide Example 9 as an amine-TFA salt.

¹H NMR (CD₃OD): δ ppm 1.30 (d, J=5.38 Hz, 6H), 1.42 (d, J=14.67 Hz, 1H), 1.63 (m, 1H), 2.05 (m, 1H), 2.44 (d, J=14.67 Hz, 1H), 3.25 (s, 1H), 3.47 (d, J=13.21 Hz, 1H), 3.62 (m, 2H), 4.24 (s, 2H), 4.61 (m, 1H), 6.98 (m, 3H), 7.33 (m, 3H), 7.45 (t, J=8.07 Hz, 1H), 7.59 (s, 1H), 7.79 (m, 1H).

EI-MS m/z: 601.20 (M+H)⁺.

The following examples were prepared starting with the 1-(3-isopropoxybenzyl)-piperidin-4-one, (Example 14-4, of International Patent Publication WO2006/044497), 1-benzyl-2-methylpiperidin-4-one or 1-(3-isopropoxybenzyl)-2-methylpiperidin-4-one (Intermediate I and II in International Patent Application WO 2007/011833, filed Jul. 14, 2006) using a procedure similar to that described for Examples 9 and 10, except that after treatment with chlorosulfonylisocyanate and water the reaction was treated with HCl to give the hydantoin product. In some instances, the racemic mixture was resolved via chiral HPLC to give the final enantiomerically pure product. Example 16 was made from palladium catalyzed coupling of N-methyl-N-prop-2-yn-1-ylmethanesulfonamide (J. Med. Chem. 1988, 31 577-582) and example 14 using Pd(tBu3P)2 , CuI, DIEA in dioxane.

| | | | Mass Spec |
|---|---|---|---|
| Ex # | Structure | Chemical name | (M + H)⁺ |
| 10 | 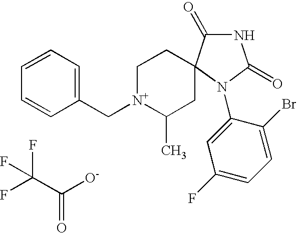 | (5R,7S)(5S,7R)8-benzyl-1-(2-bromo-5- fluorophenyl)-7-methyl-2,4-dioxo-1,3- diaza-8-azoniaspiro[4.5]decane trifluoroacetate and (5R,7R)(5S,7S)8- benzyl-1-(2-bromo-5-fluorophenyl)-7- methyl-2,4-dioxo-1,3-diaza-8- azoniaspiro[4.5]decane trifluoroacetate | 446.32 |
| 11 | 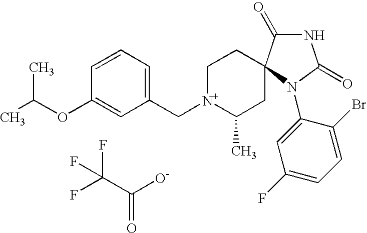 | (5R,7S)(5S,7R)1-(2-bromo-5- fluorophenyl)-8-(3-isopropoxybenzyl)- 7-methyl-2,4-dioxo-1,3-diaza-8- azoniaspiro[4.5]decane trifluoroacetate | 504.40 |
| 12 | 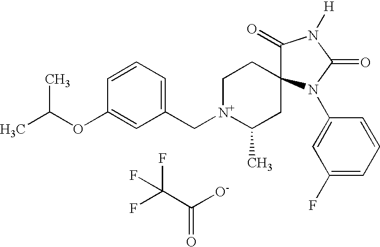 | (5R,7S)(5S,7R)1-(3-fluorophenyl)-8-(3- isopropoxybenzyl)-7-methyl-2,4-dioxo- 1,3-diaza-8-azoniaspiro[4.5]decane trifluoroacetate | 426.1 |
| 13 | 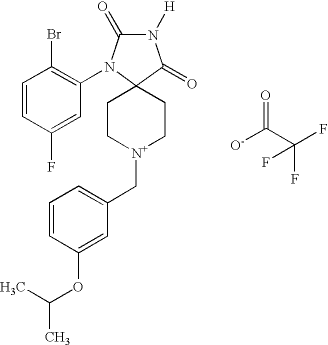 | 1-(2-bromo-5-fluorophenyl)-8-(3- isopropoxybenzyl)-2,4-dioxo-1,3-diaza- 8-azoniaspiro[4.5]decane trifluoroacetate | 490.38 |
| 14 | 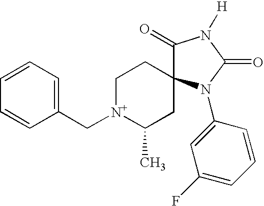 | (5R,7S)(5S,7R) 8-benzyl-1-(3- fluorophenyl)-7-methyl-1,3,8- triazaspiro[4.5]decane-2,4-dione | 368.43 |
| 15 | 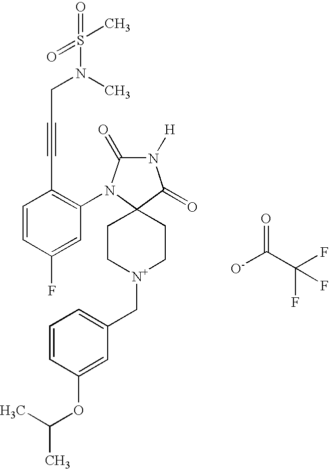 | 1-(5-fluoro-2-{3- [methyl(methylsulfonyl)amino]prop-1- yn-l-yl}phenyl)-8-(3- isopropoxybenzyl)-2,4-dioxo-1,3-diaza- 8-azoniaspiro[4.5]decane trifluoroacetate | 556.66 |
| 16 | 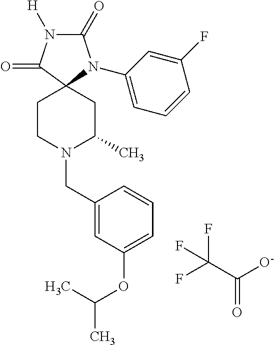 | (5R,7S)1-(3-fluorophenyl)-8-(3- isopropoxybenzyl)-7-methyl-2,4-dioxo- 1,3-diaza-8-azoniaspiro[4.5]decane trifluoroacetate | 426.51 |

### Example 16A:

### Alternative Preparation of Example 16

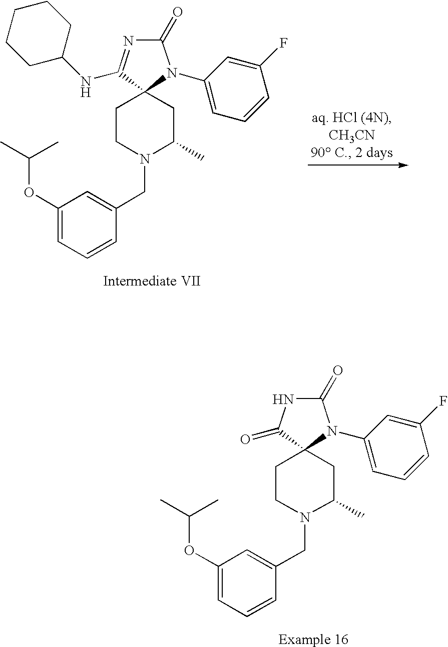

### Step 1: (5R, 7S)-1-(3-fluorophenyl)-8-(3-isopropoxybenzyl)-7-methyl-1,3,8-triazaspiro[4.5]decane-2,4-dione (Example 16)

To the solution of the bisphosphate salt of (5R, 7S)-4-(cyclohexylamino)-1-(3-fluorophenyl)-8-(3-isopropoxybenzyl)-7-methyl-1,3,8-triazaspiro[4.5]dec-3-ene-2-one (Intermediate VII, International patent application WO2007/011833, 10.0 g, 14.2 mmol) in CH₃CN (25 mL) was added aqueous HCl (4 N, 25 mL). The reaction mixture was purged with nitrogen, sealed and heated at 90° C. in a 350 mL sealed tube. After two days, the reaction mixture was concentrated, and partitioned between EtOAc and aqueous saturated NaHCO₃. The EtOAc extract was washed with brine, dried using anhydrous Na₂SO₄, concentrated under reduced pressure to obtain the desired product.

HRMS (M+1)=426.28

### Example 17

### (5R,7S)-(5S,7R)-8-benzyl-1-[4-fluoro-4′-(methylsulfonyl)-1,1′-biphenyl-2-yl]-7-methyl-1,3,8-triazaspiro[4.5]decane-2,4-dione

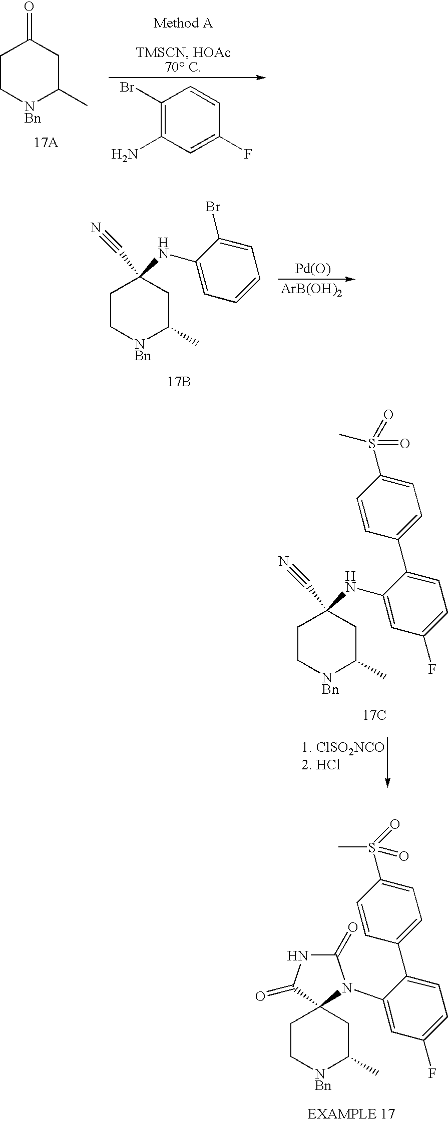

### Step 1: 17B: (5R,7S)-(5S,7R)-1-benzyl-4-(4′-bromo-4-fluoro-1,1′-biphenyl-2yl)-2-methylpiperidine-4-carbonitrile

To a solution of 1-benzyl-2-methylpiperidin-4-one (Intermediate I in International Patent Application WO 2007/011833 4.30 g, 21.2 mmol) in acetic acid (20 ml) at 0° C. were added 2-bromo-4-fluoroaniline (4.02 g, 21.2 mmol) and trimethylsilyl cyanide (2.82 ml, 21.2 mmol). The reaction was allowed to warm to rt and was then heated to 70° C. After 48 h, additional trimethylsilyl cyanide (2.82 ml, 21.2 mmol) was added to the reaction. The reaction was heated to 70° C. and allowed to stir for 7 days. The reaction was poured onto cold ammonium hydroxide and crushed ice and adjusted to pH 10. The product was extracted with dichloromethane (3×50 ml), washed with brine, dried over sodium sulfate, filtered, and concentrated under vacuum. The crude oil was purified via flash chromatography (silica, 0-20% EtOAc/hexanes) to isolate both (5R, 7R)-(5S,7S) and (5R,7S)-(5S,7R)-1-benzyl-4-(4′-bromo-4-fluoro-1,1′-biphenyl-2-yl)-2-methylpiperidine-4-carbonitrile.

¹H NMR (400 MHz, CDCl₃) δ 7.39 (dd, J=8.8, 6.0 Hz, 1H), 726 (m, 5H), 6.84 (dd, J=10.8, 2.8 Hz, 1H), 6.44 (td, J=8.2, 2.4 Hz, 1H), 4.48 (s, 1H), 4.09 (d, J=14.4 Hz, 1H), 3.08 (d, J=13.6 Hz, 1H), 2.64 (m, 1H), 2.55 (m, 1H), 2.32 (dt, J=14.0, 2.8 Hz, 1H), 2.27 (dt, J=11.5, 2.9 Hz, 1H), 2.08 (m, 3H), 1.22 (d, J=6.4 Hz, 3H).

LCMS (M+H) 401.9.

### Step 2: 17C: (5R,7S)-(5S,7R)-1-benzyl-4-[4-fluoro-4′-(methylsulfonyl)-1,1′-biphenyl-2-yl]-2-methylpiperidine-4-carbonitrile

To a solution of (5R,7S)-(5S,7R)-1-benzyl-4-(4′-bromo-4-fluoro-1,1′-biphenyl-2-yl)-2-methylpiperidine-4-carbonitrile (17B, 100 mg, 0.25 mmol) in DMF/water (80/20 v/v, 0.6 ml) under a nitrogen atmosphere were added 4-(methanesulfonyl)phenylboronic acid (74.7 mg, 0.37 mmol), tris(4,6-dimethyl-3-sulfanatophenyl)phosphine trisodium salt hydrate (24.4 mg, 0.04 mmol), palladium(II)acetate (2.8 mg, 0.01 mmol), and diisopropylamine (0.1 ml, 0.75 mmol). The reaction mixture was vortexed briefly to dissolve the catalyst and was stirred at 40° C. for 18 h. The reaction was purified via reverse phase chromatography to yield (5R,7S)-(5S,7R)-1-benzyl-4-[4-fluoro-4′-(methylsulfonyl)-1,1′-biphenyl-2-yl]-2-methylpiperidine-4-carbonitrile.

¹H NMR (400 MHz, CDCl₃) δ 7.96 (d, J=8.0 Hz, 2H), 7.47 (d, J=8.0 Hz, 2H), 7.32 (m, 3H), 7.25 (m, 2H), 7.04 (dd, J=8.4, 6.4 Hz, 1H), 6.85 (dd, J=11.2, 2.4 Hz, 1H), 6.62 (td, J=8.2, 2.3 Hz, 1H), 3.97 (d, J=13.6 Hz, 1H), 3.68 (s, 1H), 3.23 (d, J=13.2 Hz, 1H), 3.11 (s, 3H), 2.62 (dt, J=12.3, 3.1 Hz, 1H), 2.24 (m, 3H), 2.10 (dt, J=12.9, 3.7 Hz, 1H), 1.99 (m, 2H), 1.20 (d, J=5.6 Hz, 3H).

LCMS (M+H) 478.0.

### Step 3: Example 17

### (5R,7S)-(5S,7R)-8-benzyl-1-[4-fluoro-4′-(methylsulfonyl)-1,1′-biphenyl-2-yl]-7-methyl-1,3,8-triazaspiro[4.5]decane-2,4-dione

To a solution of (5R,7S)-(5S,7R)-1-benzyl-4-[4-fluoro-4′-(methylsulfonyl)-1,1′-biphenyl-2-yl]-2-methylpiperidine-4-carbonitrile (17C, 19.0 mg, 0.04 mmol) in dichloromethane (0.5 ml) was added chlorosulfonyl isocyanate (5.7 mg, 0.04 mmol) portion-wise. The reaction was allowed to stir at rt for 1 h and then concentrated. The residue was dissolved in 1N HCl, and the reaction was heated at 100° C. for 1 h. The reaction mixture was cooled to rt and adjusted to pH 5.5 by the addition of 5N NaOH. The crude material was purified via reverse phase chromatography to yield (5R,7S),(5S,7R)-8-benzyl-1-[4-fluoro-4′-(methylsulfonyl)-1,1′-biphenyl-2-yl]-7-methyl-1,3,8-triazaspiro[4.5]decane-2,4-dione.

¹H NMR (400 MHz, CDCl₃) δ 8.00 (d, J=8.4 Hz, 1.1H), 7.92 (d, J=8.4 Hz, 0.9H), 7.61 (d, J=8.4 Hz, 1.1H), 7.52 (d, J=8.4 Hz, 0.9H), 7.37 (dd, J=8.8, 6.0 Hz, 0.7H), 7.26 (m, 5H), 7.09 (m, 2.3H), 3.80 (d, J=9.6 Hz, 0.4H), 3.76 (d, J=14.8 Hz, 0.6H), 3.64 (s, 1H), 3.15 (d, J=14.0 Hz, 0.4H), 3.12 (s, 18H), 3.08 (s, 1.2H), 3.02 (d, J=13.6 Hz, 0.6H), 2.56 (m, 0.8H), 2.08 (m, 0.9H), 1.60 (t, J=13.2 Hz, 1.4H), 1.49 (m, 0.6H), 1.13 (d, J=5.6 Hz, 1.3H), 0.78 (d, J=6.4 Hz, 1.7H), 0.55 (m, 1H).

HRMS (ES, M+H), calcd. for C₂₈H₂₈FN₃O₄S: 522.1858, found: 522.1844.

### Example 18

### (5R,7S)-1-(3-fluorophenyl)-7-methyl-8-[(2′-methylbiphenyl-3-yl)methyl]-1,3,8-triazaspiro[4.5]decane-2,4-dione

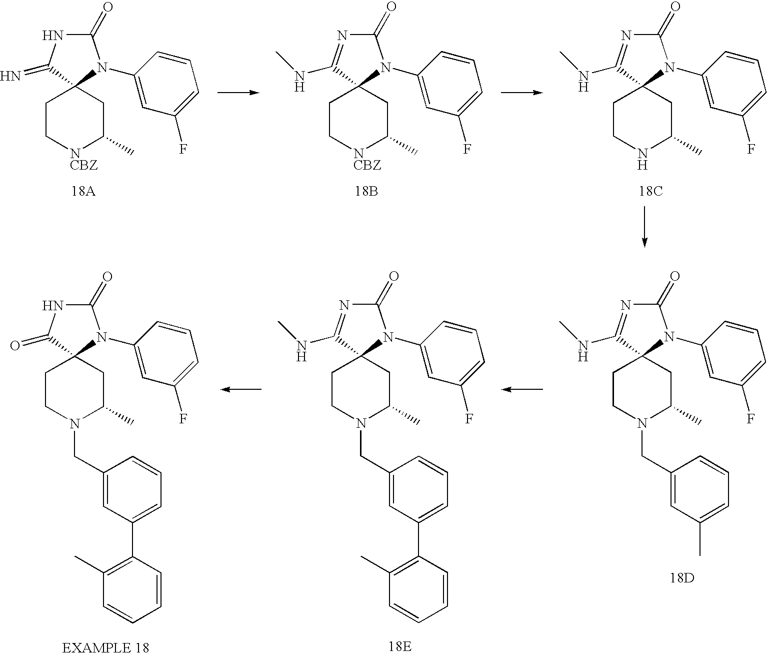

### Step 1: Benzyl (5R,7S)-1-(3-fluorophenyl)-7-methyl-4-(methylamino)-2-oxo-1,3,8-triazaspiro[4.5]dec-3-ene-8-carboxylate (18B)

To a flask containing benzyl (5R,7S)-4-amino-1-(3-fluorophenyl)-7-methyl-2-oxo-1,3,8-triazaspiro[4.5]dec-3-ene-8-carboxylate (343 mg, 0.86 mmol; described in International Patent Application WO 2007/011833 Example 11, Intermediate 11-6) was added methylamine (2.0 M THF, 4.3 mL, 8.6 mmol). The vessel was sealed and placed in a 70° C. oil bath and stirred overnight. The reaction was diluted with aqueous NaHCO₃ and extracted with EtOAc (three times). The combined organic layers were washed with brine, isolated and subsequently dried over Na₂SO₄. Evaporation of solvent and further drying under vacuum gave crude product that was used in the next step.

LCMS [M+H]=411.2

### Step 2: (5R,7S)-1-(3-fluorophenyl)-7-methyl-4-(methylamino)-1,3,8-triazaspiro[4.5]dec-3-en-2-one (18C)

The product from step 1 above, benzyl (5R,7S)-1-(3-fluorophenyl)-7-methyl-4-(methylamino)-2-oxo-1,3,8-triazaspiro[4.5]dec-3-ene-8-carboxylate (370 mg, 0.86 mmol) was dissolved in 8 mL MeOH. The solvent was degassed with a nitrogen flow for 10 min. and Pd(OH)₂ (30 mg, 20% wt. Pd) added. The mixture was purged with a hydrogen balloon for 10 min. and then maintained under atmospheric hydrogen at rt overnight with stirring. The reaction was then filtered over Celite, the cake rinsed with EtOAc and the filtrate concentrated to dryness under reduced pressure to give after drying under vacuum the product as a white solid.

LCMS [M+H]=291.2

### Step 3: (5R,7S)-1-(3-fluorophenyl)-8-(3-iodobenzyl)-7-methyl-4-(methylamino)-1,3,8-triazaspiro[4.5]dec-3-en-2-one (18D)

(5R,7S)-1-(3-fluorophenyl)-7-methyl-4-(methylamino)-1,3,8-triazaspiro[4.5]dec-3-en-2-one (250 mg, 0.86 mmol) from step 2 above was dissolved in DMSO (8.0 mL). The flask was charged with K₂CO₃ (594 mg, 4.30 mmol) and 3-iodo-benzylbromide (255 mg, 0.86 mmol). The mixture was then sealed with a septum and placed in 50° C. oil bath and allowed to stir overnight. The mixture was diluted with water and extracted with EtOAc (three times). The combined organic layers were washed with aqueous LiCl (three times), followed by brine and then dried over Na₂SO₄. Solvent removal under reduced pressure gave crude product. Purification over silica via automated flash chromatography (0 to 20% MeOH/CH₂Cl₂ over 20 min.) gave after solvent removal the product as a white solid: LCMS [M+H]=507.3

### Step 4: (5R,7S)-1-(3-fluorophenyl)-7-methyl-4-(methylamino)-8-[(2′-methylbiphenyl-3-yl)methyl]-1,3,8-triazaspiro[4.5]dec-3-en-2-one (18E)

A Biotage microwave vial was charged with intermediate (5R,7S)-1-(3-fluorophenyl)-8-(3-iodobenzyl)-7-methyl-4-(methylamino)-1,3,8-triazaspiro[4.5]dec-3-en-2-one (60 mg, 0.118 mmol) from step 3 above, PdCl₂dppf (4.3 mg, 0.01 mmol) and 2-tolylboronic acid (21 mg, 0.15 mmol). The vial was sealed and put under a nitrogen atmosphere. To the solids was added aqueous 1.5M K₂CO₃ (0.24 mL, 0.35 mmol) and degassed THF (0.7 mL). The mixture was briefly vortexed and heated in an Optimizer microwave for 5 min. at 120° C. The reaction enclosure was removed and the reaction diluted with EtOAc and water. The organic layer was washed with brine, dried over Na₂SO₄ and the solvent removed under reduce pressure. Purification over silica via automated flash chromatography (0 to 10% MeOH/CH₂Cl₂) afforded, after solvent removal under reduced pressure, the product as a white solid:

LCMS [M+H]=471.2

### Step 5: (5R,7S)-1-(3-fluorophenyl)-7-methyl-8-[(2′-methylbiphenyl-3-yl)methyl]-1,3,8-triazaspiro[4.5]decane-2,4-dione (Example 18)

In a scintillation vial (5R,7S)-1-(3-fluorophenyl)-7-methyl-4-(methylamino)-8-[(2′-methylbiphenyl-3-yl)methyl]-1,3,8-triazaspiro[4.5]dec-3-en-2-one (15 mg, 0.031 mmol) from step 4 above was dissolved in THF (1.0 mL). To this 1N HCL (3 mL) was added and the mixture sealed and heated in a 80° C. oil bath until disappearance of starting material as evident by LCMS. The mixture was then concentrated to dryness and purified by RP-HPLC using a TFA buffered solvent system to give, after lyophilization, Example 18 as a TFA salt.

¹H NMR (400 MHz, CD₃OD, Hz): δ 7.5-7.1 (m, 12H), 4.35 (AB, J=15.2 Hz, 2H), 3.46 (m, 1H), 2.90 (m, 1H), 2.63 (m, 1H), 2.53-2.30 (m, 4H), 2.21 (s, 3H), 1.56 (d, J=6.0 Hz, 3H).

LCMS [M+H]=458.0;

### Example 19

### 1-(3-fluorophenyl)-8-[(2′-methylbiphenyl-3-yl)methyl]-3-(pyridin-2-ylmethyl)-1,3,8-triazaspiro[4.5]decan-2-one

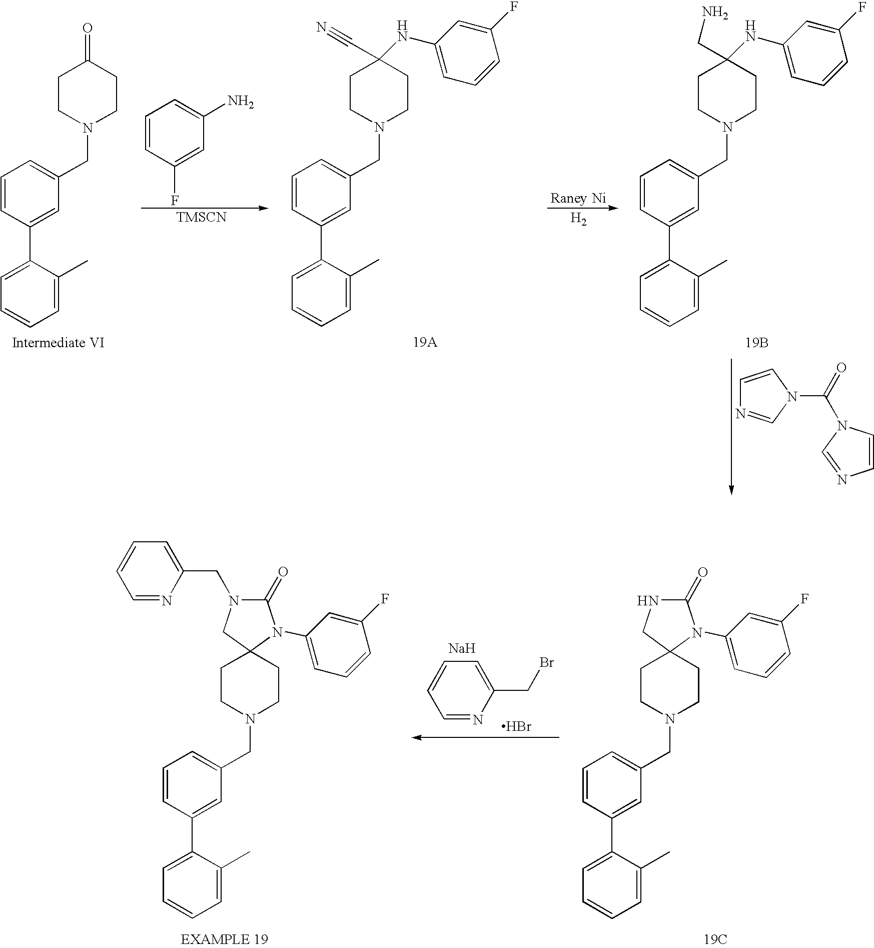

### Step 1: 4-[4(3-fluorophenyl)amino]-1-[(2′-methylbiphenyl-3-yl)methyl]piperidine-4-carbonitrile (19A)

4-[(3-fluorophenyl)amino]-1-[(2′-methylbiphenyl-3-yl)methyl]piperidine-4-carbonitrile was prepared from 1-[(2′-methylbiphenyl-3-yl)methyl]piperidin-4-one (intermediate VI, 1.3 g, 4.5 mmol), 3-fluoroaniline (0.4 ml, 4.5 mmol), and trimethylsilyl cyanide (0.6 ml, 4.5 mmol) in a manner similar to 17B from Example 17.

LCMS (M+H) 400.1.

### Step 2: 4-(aminomethyl)-N-(3-fluorophenyl)-1-[(2′-methylbiphenyl-3-yl)methyl]piperidin-4-amine (19B)

To a suspension of 4-[(3-fluorophenyl)amino]-1-[(2′-methylbiphenyl-3-yl)methyl]piperidine-4-carbonitrile (19A, 700 mg, 1.75 mmol) in ethanol (50 ml) was added a small amount of Raney Nickel (in water). The reaction was placed under an atmosphere of hydrogen and allowed to stir at rt for 5 days. The reaction was filtered, the filtrate was concentrated under vacuum, and the crude material was purified via reverse phase chromatography. The product was dissolved in dichloromethane, neutralized with excess potassium carbonate, and filtered. The filtrate was concentrated under vacuum to yield 4-(aminomethyl)-N-(3-fluorophenyl)-1-[(2′-methylbiphenyl-3-yl)methyl]piperidin-4-amine.

¹H NMR (400 MHz, DMSO) δ 7.38 (m, 1H), 7.22 (m, 6H), 7.00 (m, 1H), 6.51 (m, 2H), 6.28 (m, 1H), 5.24 (s, 1H), 4.11 (m, 1H), 3.49 (s, 2H), 3.15 (m, 2H), 2.71 (s, 2H), 2.29 (t, J=9.8 Hz, 2H), 2.21 (s, 3H), 2.03 (m, 1H), 1.91 (d, J=13.6 Hz, 2H), 1.56 (t, J=10.5 Hz, 2H).

LCMS (M+H) 404.3.

### Step 3: 1-(3-fluorophenyl)-8-[(2′-methylbiphenyl-3-yl)methyl]-1,3,8-triazaspiro[4.5]decan-2-one (19C)

1-(3-fluorophenyl)-8-[(2′-methylbiphenyl-3-yl)methyl]-1,3,8-triazaspiro[4.5]decan-2-one was prepared from 4-(aminomethyl)-N-(3-fluorophenyl)-1-[(2′-methylbiphenyl-3-yl)methyl]piperidin-4-amine (19B, 265 mg, 0.66 mmol), N,N′-carbonyldiimidazole (107 mg, 0.66 mmol), and triethylamine (140 μl, 0.99 mmol) in a manner similar to Example 20.

LCMS (M+H) 430.2.

### Step 4: 1-(3-fluorophenyl)-8-[(2′-methylbiphenyl-3-yl)methyl]-3-(pyridin-2-ylmethyl)-1,3,8-triazaspiro[4.5]decan-2-one (Example 22)

1-(3-fluorophenyl)-8-[(2′-methylbiphenyl-3-yl)methyl]-3-(pyridin-2-ylmethyl)-1,3,8-triazaspiro[4.5]decan-2-one was prepared from 1-(3-fluorophenyl)-8-[(2′-methylbiphenyl-3-yl)methyl]-1,3,8-triazaspiro[4.5]decan-2-one (19C, 30 mg, 0.07 mmol) and 2-(bromomethyl)pyridine hydrobromide (12 mg, 0.07 mmol) in a manner similar to Example 21.

¹H NMR (400 MHz, CDCl₃) δ 8.57 (d, J=4.8 Hz, 1H), 7.69 (td, J=7.6, 1.7 Hz, 1H), 7.33 (m, 3H), 7.21 (m, 8H), 7.00 (m, 2H), 6.93 (d, J=9.8, 2.1, 1H), 4.60 (s, 2H), 3.46 (s, 2H), 3.38 (s, 2H), 2.85 (d, J=11.7 Hz, 2H), 2.22 (s, 3H), 1.99 (t, J=11.4 Hz, 2H), 1.84 (td, J 12.5, 3.8 Hz, 2H), 1.70 (d, J=12.1 Hz, 2H).

LCMS (M+H) 521.4.

### Examples 20-22

### 4′-fluoro-2′-[8-(3-isopropoxybenzyl)-2-oxo-1,3,8-triazaspiro[4.5]dec-1-yl}-N,N-dimethylbiphenyl-4-sulfonamide (Example 20)

### 1-{4′-[(dimethylamino)sulfonyl]-4-fluorobiphenyl-2-yl}-8-(3-isopropoxybenzyl)-2-oxo-3-propyl-1,3-diaza-8-azoniaspiro[4.5]decane trifluoroacetate (Example 21)

### 1-{4′-[(dimethylamino)sulfonyl]-4-fluorobiphenyl-2-yl}-8-(3-isopropoxybenzyl)-3-methyl-2-oxo-1,3-diaza-8-azoniaspiro[4.5]decane trifluoroacetate (Example 22)

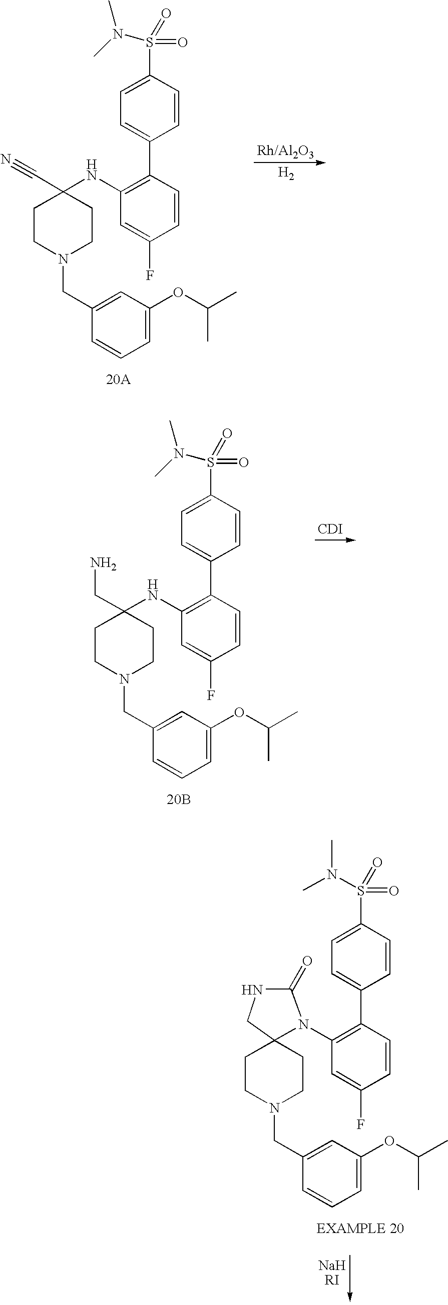

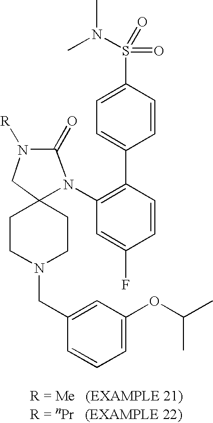

Compound 20A (Scheme 3, intermediate 3-B, 4.110 g, 0.00746 mol), Rh/Al₂O₃ (1.350 g) and 6N ammonia in MeOH (50 ml) were mixed together. The mixture was allowed to be hydrogenated in the Parr shaker at 45 psi overnight. Filtrated with celite and concentrated to afford crude Compound 20B.

EI-MS m/z: 555 (M+H)⁺ at 1.50.

Crude Compound 20B (0.310 g, 0.559 mmol), carbonyldiimidazole (0.136 g, 1.5 eq) and DCM (2 ml) were mixed and heated at 55° C. overnight. The mixture was concentrated and purified by preparative HPLC to afford Example 20.

EI-MS m/z: 581 (M+H)⁺ at 1.63.

To a stirred solution of Example 21 (0.025 g, 0.0431 mmol) in DMF (1.5 ml), was added NaH (60% dispersion in mineral oil) (0.013 g, 10 eq) at 0° C. MeI (0.0026 ml, 1.0 eq) was then added. The reaction was allowed to warm to rt and stirred for another 30 min before being quenched with saturated aqueous NH₄Cl solution. The mixture was extracted with Et₂O twice and the combined organic extracts were washed with brine twice, dried with MgSO₄, and purified by preparative HPLC to afford Example 21.

EI-MS m/z: 595 (M+H)⁺ at 1.68.

To a stirred solution of Example 24 (0.025 g, 0.0431 mmol) in DMF (1.5 ml), was added NaH (60% dispersion in mineral oil) (0.013 g, 10 eq) at 0° C. MeI (0.0026 ml, 1.0 eq) was then added and the reaction was allowed to warm to rt and stirred for another 30 min. The reaction was quenched with saturated aqueous NH₄Cl solution, extracted with Et₂O twice, washed with brine twice, dried with MgSO₄, concentrated and purified by preparative HPLC to afford Example 22.

EI-MS m/z: 623 (M+H)⁺ at 1.74.

The following examples were prepared starting with 1-(3-isopropoxybenzyl)-piperidin-4-one, (Example 14-4, in International Patent Application WO 2006/044497), 1-benzyl-2-methylpiperidin-4-one (Intermediate I in International Patent Application WO 2007/011833) or 1-(3-sec-butoxybenzyl)-2-methylpiperidin-4-one (also from International Patent Application WO 2007/011833) using a procedure similar to that described for Examples 3 and 20. The 3-fluoro-2-(4-methyanesulfonylphenyl)analine used for Example 23 was prepared in a manner similar to that described for Intermediate V. For Examples 25-27, the benzyl group of Example 24 was removed with catalyst and hydrogen, and the piperidine alkylated with 1-(chloromethyl)-3-isopropoxybenzene (International Patent Application WO 2007/011833 Example 11, Intermediate 11-10C), 1-(chloromethyl)-3-{[(1R)-1-methylpropyl]oxy}benzene (above patent, Intermediate III), or 1-(chloromethyl)-3-[(1R)-2-methoxy-1-methylethoxy]benzene, which was made in a manner similar to that described for the above Intermediate III.

| | | | Mass Spec |
|---|---|---|---|
| Ex # | Structure | Chemical name | (M + H)⁺ |
| 23 | 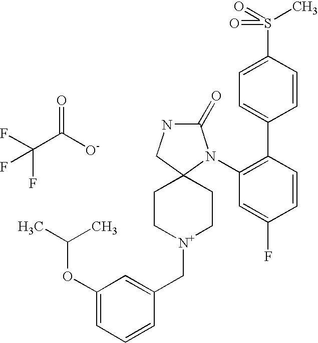 | 1-[4-fluoro-4′- (methylsulfonyl)biphenyl- 2-yl]-8-(3- isopropoxybenzyl)-2-oxo- 1,3-diaza-8- azoniaspiro[4.5]decane trifluoroacetate | 551.69 |
| 24 | 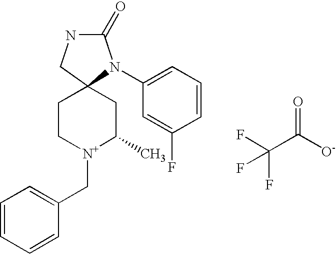 | (5R,7S)-(5S,7R)-8- benzyl-1-(3- fluorophenyl)-7-methyl-2- oxo-1,3-diaza-8- azoniaspiro[4.5]decane trifluoroacetate | 353.44 |
| 25 | 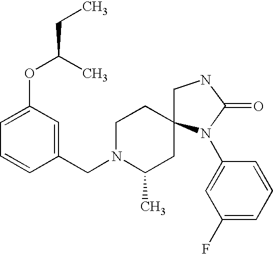 | (5R,7S)-(5S,7R)-1-(3- fluorophenyl)-7-methyl-8- (3-{[(1R)-1- methylpropyl]oxy}benzyl)- 1,3,8- triazaspiro[4.5]decan-2- one 2 diastereomers. | 426.55 |
| 26 | 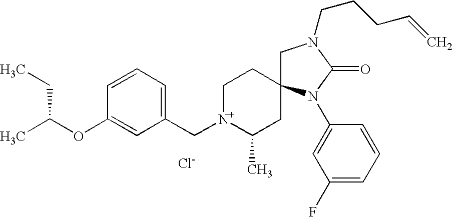 | (5R,7S)-(5S,7R)-1-(3- fluorophenyl)-7-methyl-8- (3-{[(1R)-1- methylpropyl]oxy}benzyl)- 2-oxo-3-pent-4-en-1-yl- 1,3-diaza-8- azoniaspiro[4.5]decane chloride | 493.67 |
| 27 | 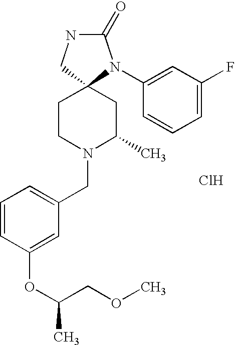 | (5R,7S)-1-(3- fluorophenyl)-8-{3-[(1R)- 2-methoxy-1- methylethoxy]benzyl}-7- methyl-1,3,8- triazaspiro[4.5]decan-2- one hydrochloride | 441.55 |

### Example 28

### 8-benzyl-1-(3-fluorophenyl)-7-methyl-2-oxo-3-oxa-1-aza-8-azoniaspiro[4.5]decane trifluoroacetate

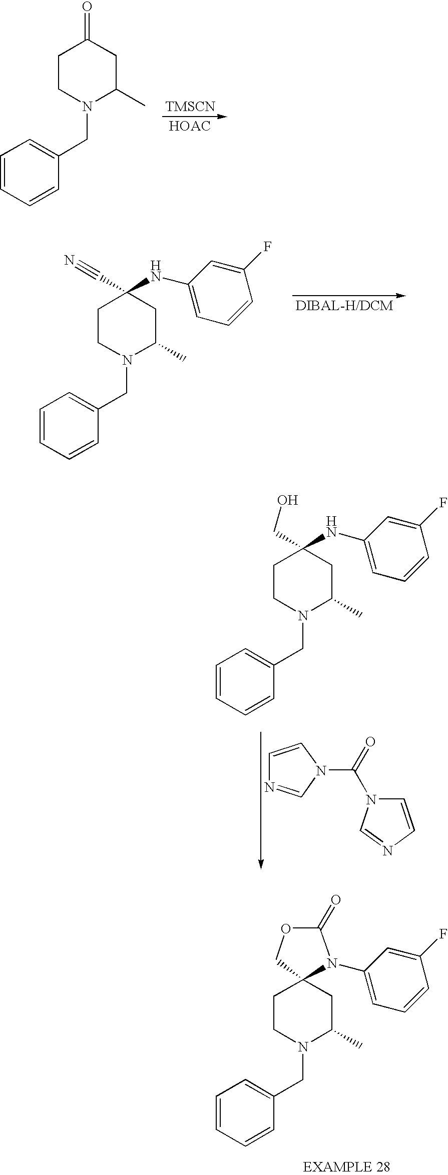

### Step 1: 1-benzyl-4-[(3-fluorophenyl)amino]-2-methylpiperidine-4-carbonitrile

To a mixture of 1-benzyl-2-methylpiperidin-4-one (Intermediate I, from International Patent Application WO 2007/011833, filed Jul. 14, 2006, 3.0 g, 14.8 mmol) in HOAc (15 mL) was added 3-fluoroaniline (1.64 g, 14.8 mmol) and TMSCN(1.97 mL, 14.8 mmol). After stirring at rt overnight the reaction mixture was poured into 15 mL NH₄OH and 15 g ice. The pH of the solution was adjusted to pH=8 by additional NH₄OH. The resulting solution was extracted with CHCl₃ three times, dried with MgSO₄ and concentrated.

LRMS (M+1)=324

### Step 2: 1-benzyl-4-[(3-fluorophenyl)amino]-2-methylpiperidine-4-carbonitrile

(2R,4R)-1-benzyl-4-[(3-fluorophenyl)amino]-2-methylpiperidine-4-carbonitrile (4.77 g, 14.8 mmol) was dissolved in MeOH (100 mL) and added to a sealed tube. After heating overnight the reaction mixture was concentrated and purified on a silica gel cartridge (5% EtOAc/hexanes to 30% EtOAc/hexanes) to afford the desired product. The rest of the mixture was heated again and chromatographed to afford the desired product.

LRMS (M+1)=324

### Step 3: 1-benzyl-4-[(3-fluorophenyl)amino]-2-methylpiperidin-4-yl}methanol

To a solution of (2S,4R)-1-benzyl-4-[(3-fluorophenyl)amino]-2-methylpiperidine-4-carbonitrile (1.29 g, 3.99 mmol) in methylene chloride added DIBAL in cyclohexane (10.88 mL, 11.97 mmol). After stirring for 3 h the reaction was quenched with water then concentrated H₂SO₄. Additional DCM was added and the solution was warmed to rt. The layers were separated and the aqueous layer was made basic by the addition of NaOH. The aqueous phase was extracted with DCM three times, dried with MgSO₄ and concentrated. To the resulting oil was added dry MeOH (10 mL) and excess NaBH₄ (0.754 g, 19.9 mmol) at 0° C. After stirring for 72 h the reaction was quenched with sodium bicarbonate solution and diluted with DCM. The layers were separated and extracted with DCM to afford a mixture of the desired alcohol product as well as amine resulting from incomplete hydrolysis of the imine precursor. The mixture of both intermediates was used directly in the next reaction.

LRMS (M+1)=329

### Step 4: 8-benzyl-1-(3-fluorophenyl)-7-methyl-2-oxo-3-oxa-1-aza-8-azoniaspiro[4.5]decane trifluoroacetate

Carbonyldiimidazole (0.851 g, 5.25 mmol) was added to a solution of {(2S,4R)-1-benzyl-4-[(3-fluorophenyl)amino]-2-methylpiperidin-4-yl}methanol (0.862 g, 2.63 mmol) in THF. The mixture was cooled to 0° C. and NaH (2.63 mmol) was added. After 4 h at rt, the reaction was quenched with NH₄Cl and made slightly basic with 1M NaOH. The basic solution was extracted with DCM three times, dried with MgSO₄ and concentrated under vacuum. Purification by reverse phase HPLC afforded the desired compound.

¹H NMR (CD₃OD): δ 7.55-7.06 (m, 9H), 4.50 (m, 1H), 4.25 (m, 2H), 3.80 (m, 1H), 3.48-3.12 (m, 3H), 2.65 (m, 2H), 2.09 (m, 2H), 1.50 (m, 3H).

HRMS=355.18

### Example 29

### -(5R,7S)-1-(3-fluorophenyl)-8-(3-isopropoxybenzyl)-3-[2-(4-methoxyphenyl)ethyl]-7-methyl-2,4-dioxo-1,3-diaza-8-azoniaspiro[4.5]decane trifluoroacetate

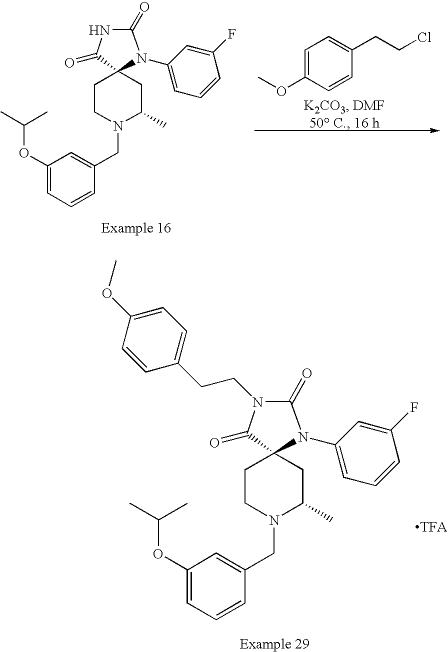

### Step 1: (5R,7S)-1-(3-fluorophenyl)-8-(3-isopropoxybenzyl)-3-[2-(4-methoxyphenyl)ethyl]-7-methyl-2,4-dioxo-1,3-diaza-8-azoniaspiro[4.5]decane trifluoroacetate Example 29

To a mixture of 1-(2-chloroethyl)-4-methoxybenzene (0.034 g, 0.20 mmol) and anhydrous K₂CO₃ (0.042 g, 0.30 mmol) was added a solution of (5R,7S)-1-(3-fluorophenyl)-8-(3-isopropoxybenzyl)-7-methyl-1,3,8-triazaspiro[4.5]decane-2,4-dione (Example 16, 0.043 g, 0.10 mmol) in anhydrous DMF (1 mL). After stirring at 50° C. for 16 hours, the reaction was cooled and quenched with aqueous NH₄Cl.

The resulting mixture was extracted with EtOAc (two times) and the EtOAc extract was concentrated under reduced pressure. The crude material was purified by reverse phase preparative HPLC to afford the desired compound as a TFA salt. For NMR analysis, the sample was dissolved in CDCl₃ and converted to free base by NH₃ vapor.

¹H NMR (600 MHz, CDCl₃): δ 7.36 (q, J=5.97 Hz, 1H), 7.12-7.14 (m, 4H), 6.94 (dd, J=8.02 Hz, 1H), 6.85-6.86 (m, 3H), 6.70-6.73 (m, 3H), 4.51 (m, 1H), 3.80-3.82 (m, 3H), 3.78 (s, 3H), 2.95 (t, J=7.56 Hz, 2H), 2.86 (d, J=14.0 Hz, 1H), 2.57 (m, 1H), 2.17-2.18 (m, 2H), 2.01 (t, J=14.0 Hz, 1H), 1.68-1.73 (m, 3H), 1.31 (d, J=6.37 Hz, 6H), 1.06 (d, J5.73 Hz, 3H).

LRMS (M+1)=560.29

The following examples were prepared using a procedure similar to that described for Example 29. In some instances, the compound was isolated as the TFA salt after chromatography, and in some cases the compound was then isolated as the free base by extraction from a suitable aqueous base like bicarbonate solution with a suitable organic solvent like methylene chloride. The free base could then be transformed to the hydrochloride salt by treatment with an ether solution of HCl. Examples 29-21 and 29-22 were prepared using the esterified form of the alkylating agent, and the ester product was then saponified to the acid.

| | | | Mass Spec |
|---|---|---|---|
| Ex # | Structure | Chemical Name | (M + H)⁺ |
| 29-1 | 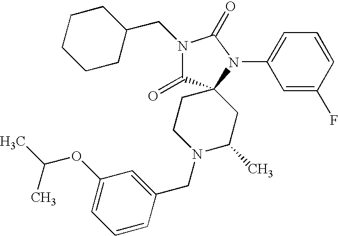 | (5R,7S)-3-(cyclohexylmethyl)- 1-(3-fluorophenyl)-8-(3- isopropoxybenzyl)-7-methyl- 1,3,8-triazaspiro[4.5]decane- 2,4-dione | 522.3 |
| 29-2 | 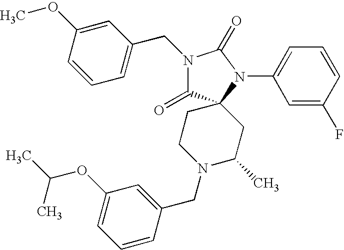 | (5R,7S)-1-(3-fluorophenyl)-8- (3-isopropoxybenzyl)-3-(3- methoxybenzyl)-7-methyl-1,3,8- triazaspiro[4.5]decane-2,4-dione | 546.3 |
| 29-3 | 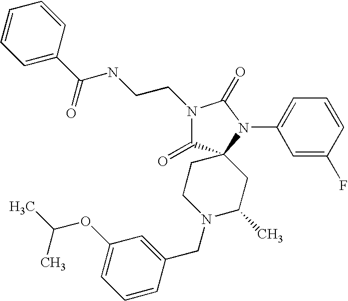 | N-{2-[(5R,7S)-1-(3- fluorophenyl)-8-(3- isopropoxybenzyl)-7-methyl- 2,4-dioxo-1,3,8- triazaspiro[4.5]dec-3- yl]ethyl}benzarnide | 573.3 |
| 29-4 | 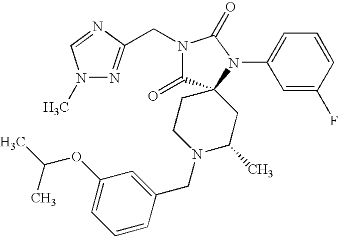 | (5R,7S)-1-(3-fluorophenyl)-8- (3-isopropoxybenzyl)-7-methyl- 3-[(1-methyl-1H-1,2,4-triazol-3- yl)methyl]-1,3,8- triazaspiro[4.5]decane-2,4-dione | 520.9 |
| 29-5 | 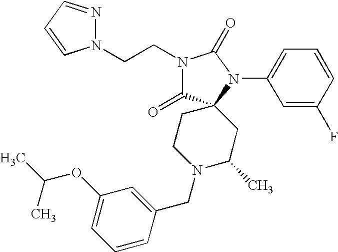 | (5R,7S)-1-(3-fluorophenyl)-8- (3-isopropoxybenzyl)-7-methyl- 3-[2-(1H-pyrazol-1-yl)ethyl]- 1,3,8-triazaspiro[4.5]decane- 2,4-dione | 520.2 |
| 29-6 | 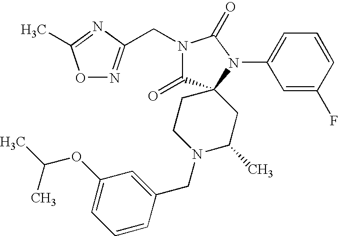 | (5R,7S)-1-(3-fluorophenyl)-8- (3-isopropoxybenzyl)-7-methyl- 3-[(5-methyl-1,2,4-oxadiazol-3- yl)methyl]-1,3,8- triazaspiro[4.5]decane-2,4-dione | 522.1 |
| 29-7 | 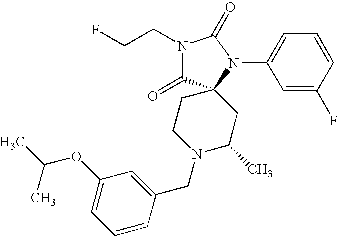 | (5R,7S)-3-(2-fluoroethyl)-1-(3- fluorophenyl)-8-(3- isopropoxybenzyl)-7-methyl- 1,3,8-triazaspiro[4.5]decane- 2,4-dione | 472.2 |
| 29-8 | 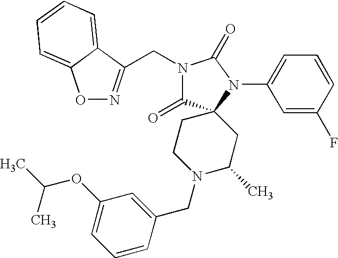 | (5R,7S)-3-(1,2-benzisoxazol-3- ylmethyl)-1-(3-fluorophenyl)-8- (3-isopropoxybenzyl)-7-methyl- 1,3,8-triazaspiro[4.5]decane- 2,4-dione | 557.3 |
| 29-9 | 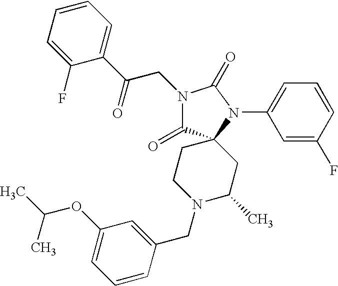 | (5R,7S)-1-(3-fluorophenyl)-3- [2-(2-fluorophenyl)-2-oxoethyl]- 8-(3-isopropoxybenzyl)-7- methyl-1,3,8- triazaspiro[4.5]decane-2,4-dione | 562.3 |
| 29-10 | 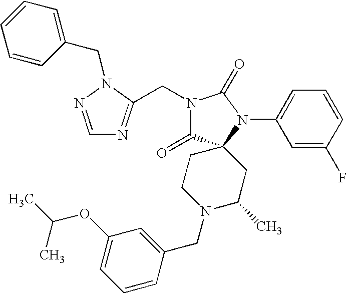 | (5R,7S)-3-[(1-benzyl-1H-1,2,4- triazol-5-yl)methyl]-1-(3- fluorophenyl)-8-(3- isopropoxybenzyl)-7-methyl- 1,3,8-triazaspiro[4.5]decane- 2,4-dione | 597.3 |
| 29-11 | 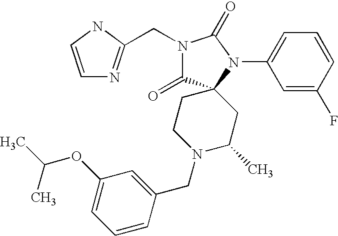 | (5R,7S)-1-(3-fluorophenyl)-3- (1H-imidazol-2-ylmethyl)-8-(3- isopropoxybenzyl)-7-methyl- 1,3,8-triazaspiro[4.5]decane- 2,4-dione | 506.3 |
| 29-12 | 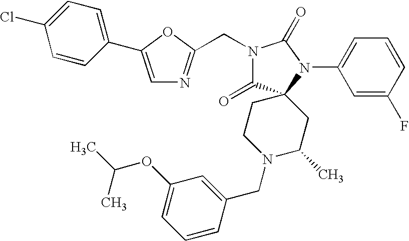 | (5R,7S)-3-{(5-(4-chlorophenyl)- 1,3-oxazol-2-yl]methyl}-1-(3- fluorophenyl)-8-(3- isopropoxybenzyl)-7-methyl- 1,3,8-triazaspiro[4.5]decane- 2,4-dione | 617.2 |
| 29-13 | 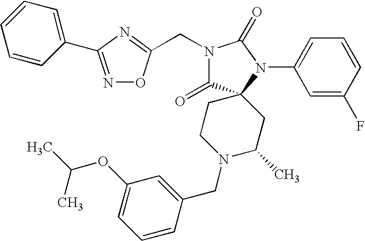 | (5R,7S)-1-(3-fluorophenyl)-8- (3-isopropoxybenzyl)-7-methyl- 3-[(3-phenyl-1,2,4-oxadiazol-5- yl)methyl]-1,3,8- triazaspiro[4.5]decane-2,4-dione | 584.3 |
| 29-14 | 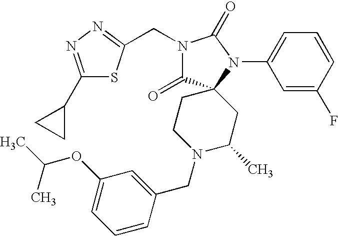 | (5R,7S)-3-[(5-cyclopropyl- 1,3,4-thiadiazol-2-yl)methyl]-1- (3-fluorophenyl)-8-(3- isopropoxybenzyl)-7-methyl- 1,3,8-triazapiro[4.5]decane- 2,4-dione | 564.3 |
| 29-15 | 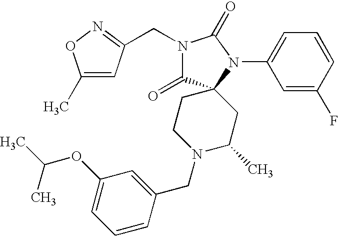 | (5R,7S)-1-(3-fluorophenyl)-8- (3-isopropoxybenzyl)-7-methyl- 3-[(5-methylisoxazol-3- yl)methyl]-1,3,8- thazaspiro[4.5]decane-2,4-dione | 521.3 |
| 29-16 | 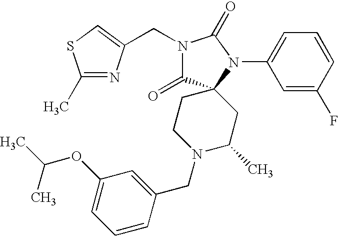 | (5R,7S)-1-(3-fluorophenyl)-8- (3-isopropoxybenzyl)-7-methyl- 3-[(2-methyl-1,3-thiazol-4- yl)methyl]-1,3,8- triazaspiro[4.5]decane-2,4-dione | 537.2 |
| 29-17 | 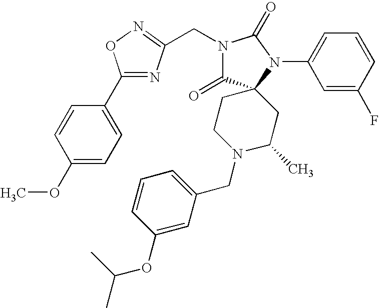 | (5R,7S)-1-(3-fluorophenyl)-8- (3-isopropoxybenzyl)-3- {[5-(4- methoxyphenyl)-1,2,4- oxadiazol-3-yl]methyl}-7- methyl-1,3,8- triazaspiro[4.5]decane-2,4-dione | 614.3 |
| 29-18 | 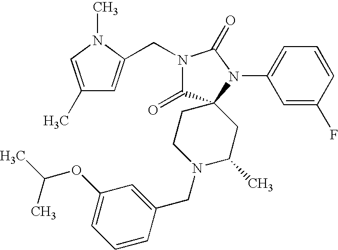 | (5R,7S)-3-[(1,3-dimethyl-1H- pyrazol-5-yl)methyl]-1-(3- fluorophenyl)-8-(3- isopropoxybenzyl)-7-methyl- 1,3,8-triazaspiro[4.5]decane- 2,4-dione | 534.3 |
| 29-19 | 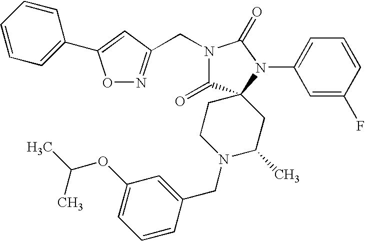 | (5R,7S)-1-(3-fluorophenyl)-8- (3-isopropoxybenzyl)-7-methyl- 3-[(5-phenylisoxazol-3- yl)methyl]-1,3,8- triazaspiro[4.5]decane-2,4-dione | 583.3 |
| 29-20 | 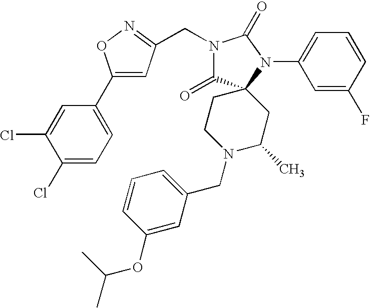 | (5R,7S)-3-{[5-(3,4- dichlorophenyl)isoxazol-3- yl]methyl}-1-(3-fluorophenyl)- 8-(3-isopropoxybenzyl)-7- methyl-1,3,8- triazaspiro[4.5]decane-2,4-dione | 651.2 |
| 29-21 | 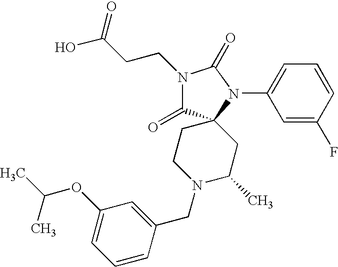 | 3-[(5R,7S)-1-(3-fluorophenyl)- 8-(3-isopropoxybenzyl)-7- methyl-2,4-dioxo-1,3,8- triazaspiro[4.5]dec-3- yl]propanoic acid | 498.0 |
| 29-22 | 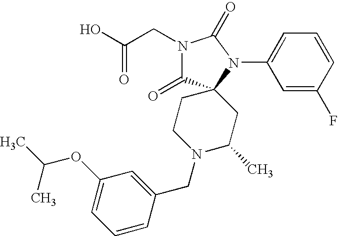 | [(5R,7S)-1-(3-fluorophenyl)-8- (3-isopropoxybenzyl)-7-methyl- 2,4-dioxo-1,3,8- triazaspiro[4.5]dec-3-yl]acetic acid | 483.8 |
| 29-23 | 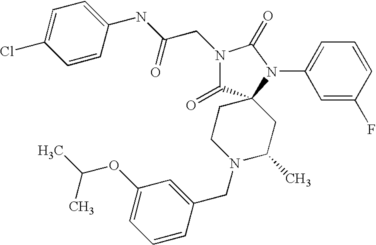 | N-(4-chlorophenyl)-2-[(5R,7S)- 1-(3-fluorophenyl)-8-(3- isopropoxybenzyl)-7-methyl- 2,4-dioxo-1,3,8- triazaspiro[4.5]dec-3- yl]acetamide | 593.2 |
| 29-24 | 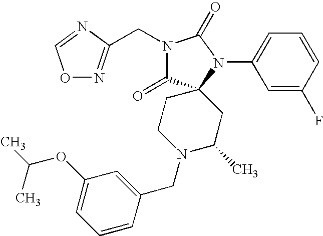 | (5R,7S)-1-(3-fluorophenyl)-8- (3-isopropoxybenzyl)-7-methyl- 3-(1,2,4-oxadiazol-3-ylmethyl)- 1,3,8-triazaspiro[4.5]decane- 2,4-dione | 508.0 |
| 29-25 | 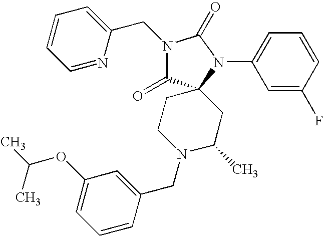 | (5R,7S)-1-(3-fluorophenyl)-8- (3-isopropoxybenzyl)-7-methyl- 3-(pyridin-2-ylmethyl)-1,3,8- triazaspiro[4.5]decane-2,4-dione | 517.3 |

### Example 30

### -(5R,7S)-1-(3-fluorophenyl)-3-(3-furylmethyl)-8-(3-isopropoxybenzyl)-7-methyl-2,4-dioxo-1,3-diaza-8-azoniaspiro[4.5]decane trifluoroacetate

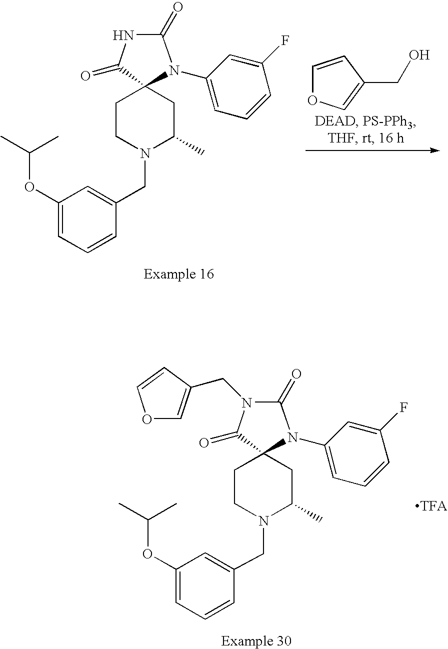

### Step 1: (5R,7S)-1-(3-fluorophenyl)-3-(3-furylmethyl)-8-(3-isopropoxybenzyl)-7-methyl-2,4-dioxo-1,3-diaza-8-azoniaspiro[4.5]decane trifluoroacetate Example 30

To a mixture of (5R,7S)-1-(3-fluorophenyl)-8-(3-isopropoxybenzyl)-7-methyl-1,3,8-triazaspiro[4.5]decane-2,4-dione (Example 16, 0.043 g, 0.10 mmol), 3-furylmethanol (0.020 g, 0.20 mmol), and PS-PPh3 (0.093 g, 0.20 mmol, resin loading 2.15 mmol/g) in anhydrous THF (1 mL) was added DEAD (0.035 g, 0.20 mmol). After stirring at room temperature for 16 hours, the reaction was diluted with THF, filtered, and the filtrate was concentrated under reduced pressure. The crude material was purified by reverse phase preparative HPLC to afford the desired compound as a TFA salt. For NMR analysis, the sample was dissolved in CD₃OD and converted to free base by NH₃ vapor.

¹H NMR (600 MHz, CD₃OD): δ 7.51 (s, 1H), 7.43 (m, 1H), 7.38 (m, 1H), 7.11-7.15 (m, 4H), 6.75 (dd, J=8.00 Hz, 2.52 Hz, 1H), 6.64-6.66 (m, 2H), 6.43 (m, 1H), 4.55 (s, 2H), 4.53 (m, 1H), 3.76 (d, J=12.9 Hz, 1H), 3.14 (d, J=12.9 Hz, 1H), 2.59 (m, 1H), 2.22-2.19 (m, 2H), 2.02-2.08 (m, 3H), 1.84 (m, 1H), 1.29 (d, J=6.11 Hz, 6H), 1.15 (d, J=6.17 Hz, 3H).

LRMS (M+1)=506.25

The following examples were prepared using a procedure similar to that described for Example 30. In some instances, the compound was isolated as the TFA salt after chromatography, and in some cases the compound was then isolated as the free base by extraction from a suitable aqueous base like bicarbonate solution with a suitable organic solvent like methylene chloride. The free base could then be transformed to the hydrochloride salt by treatment with an ether solution of HCl.

| | | | Mass Spec |
|---|---|---|---|
| Ex # | Structure | Chemical Name | (M + H)⁺ |
| 30-1 | 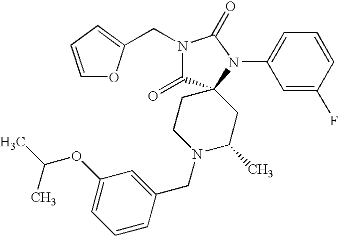 | (5R,7S)-1-(3- fluorophenyl)-3-(2- furylmethyl)-8-(3- isopropoxybenzyl)-7- methyl-1,3,8- triazaspiro[4.5]decane- 2,4-dione | 506.3 |
| 30-2 | 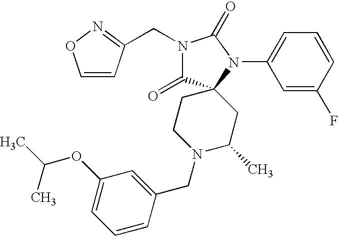 | (5R,7S)-1-(3- fluorophenyl)-8-(3- isopropoxybenzyl)-3- (isoxazol-3-ylmethyl)- 7-methyl-1,3,8- triazaspiro[4.5]decane- 2,4-dione | 507.2 |
| 30-3 | 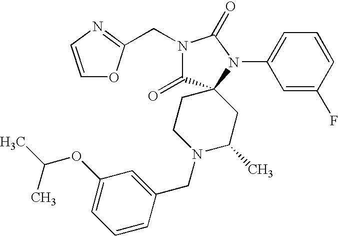 | (5R,7S)-1-(3- fluorophenyl)-8-(3- isopropoxybenzyl)-7- methyl-3-(1,3-oxazol- 2-ylmethyl)-1,3,8- triazaspiro[4.5]decane- 2,4-dione | 507.2 |
| 30-4 | 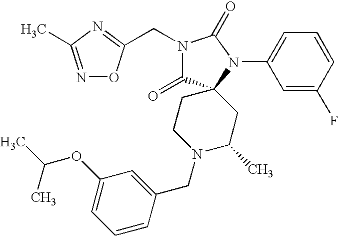 | (5R,7S)-1-(3- fluorophenyl)-8-(3- isopropoxybenzyl)-7- methyl-3-[(3-methyl- 1,2,4-oxadiazol-5- yl)methyl]-1,3,8- triazaspiro[4.5]decane- 2,4-dione | 522.3 |
| 30-6 | 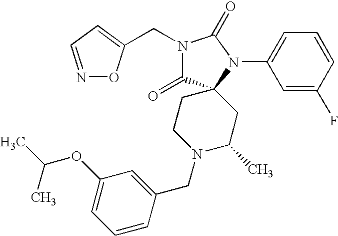 | (5R,7S)-1-(3- fluorophenyl)-8-(3- isopropoxybenzyl)-3 - (isoxazol-5-ylmethyl)- 7-methyl-1,3,8- triazaspiro[4.5]decane- 2,4-dione | 507.2 |
| 30-7 | 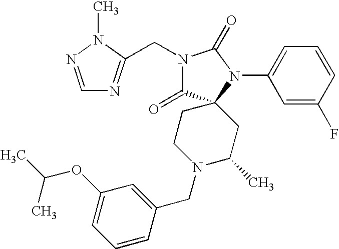 | (5R,7S)-1-(3- fluorophenyl)-8-(3- isopropoxybenzyl)-7- methyl-3-[(1-methyl- 1H-1,2,4-triazol-5- yl)methyl]-1,3,8- triazaspiro[4.5]decane- 2,4-dione | 521.3 |
| 30-8 | 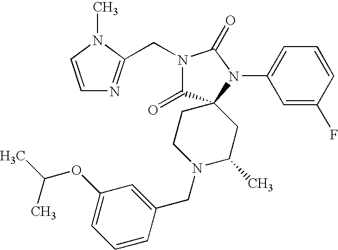 | (5R,7S)-1-(3- fluorophenyl)-8-(3- isopropoxybenzyl)-7- methyl-3-[(1-methyl- 1H-imidazol-2- yl)methyl]-1,3,8- triazaspiro [4.5]decane- 2,4-dione | 520.3 |
| 30-9 | 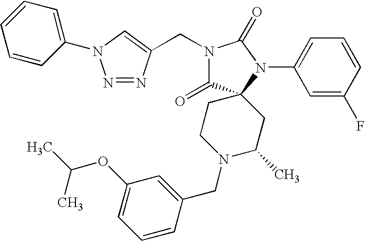 | (5R,7S)-1-(3- fluorophenyl)-8-(3- isopropoxybenzyl)-7- methyl-3-[(1-phenyl- 1H-1,2,3-triazol-4- yl)methyl]-1,3,8- triazaspiro[4.5]decane- 2,4-dione | 583.3 |
| 30-10 | 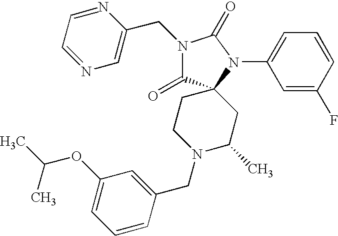 | (5R,7S)-1-(3- fluorophenyl)-8-(3- isopropoxybenzyl)-7- methyl-3-(pyrazin-2- ylmethyl)-1,3,8- triazaspiro[4.5]decane- 2,4-dione | 518.3 |
| 30- 11 | 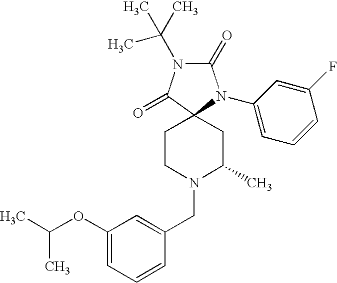 | (5R,7S)-3-tert-butyl-1- (3-fluorophenyl)-S-(3- isopropoxybenzyl)-7- methyl-1,3,8- triazaspiro[4. 5]decane- 2,4-dione | 482.0 |
| 30-12 | 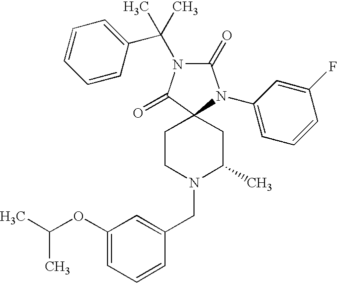 | (5R,7S)-1-(3- fluorophenyl)-8-(3- isopropoxybenzyl)-7- methyl-3-(1-methyl-1- phenylethyl)-1,3,8- triazaspiro[4.5]decane- 2,4-dione | 544.1 |
| 30-13 | 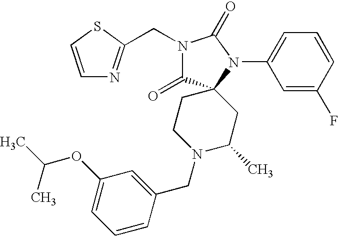 | (5R,7S)-1-(3- fluorophenyl)-8-(3- isopropoxybenzyl)-7- methyl-3-(1,3-thiazol- 2-ylmethyl)-1,3,8- triazaspiro[4.5]decane- 2,4-dione | 523.2 |
| 30-14 | 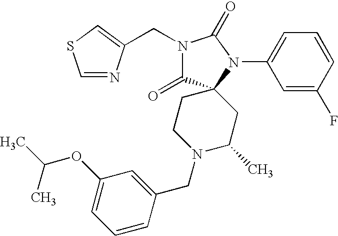 | (5R,7S)-1-(3- fluorophenyl)-8-(3- isopropoxybenzyl)-7- methyl-3-(1,3-thiazol- 4-ylmethyl)-1,3,8- triazaspiro[4.5]decane- 2,4-dione | 523.2 |
| 30-15 | 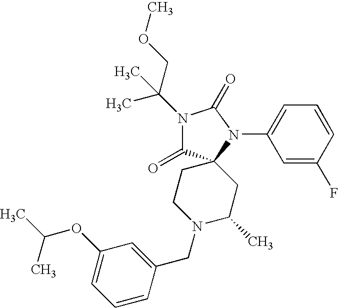 | (5R,7S)-1-(3- fluorophenyl)-8-(3- isopropoxybenzyl)-3- (2-methoxy-1,1- dimethylethyl)-7- methyl-1,3,8- triazaspiro[4.5]decane- 2,4-dione | 512.3 |

### Example 31

### (5R,7S)-1-(3-fluorophenyl)-8-(3-isopropoxybenzyl)-7-methyl-2,4-dioxo-3-phenyl-1,3-diaza-8-azoniaspiro[4.5]decane trifluoroacetate

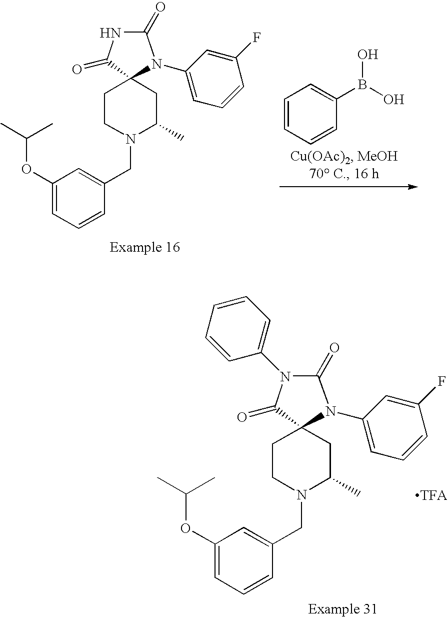

### Step 1: (5R,7S)-1-(3-fluorophenyl)-8-(3-isopropoxybenzyl)-7-methyl-2,4-dioxo-3-phenyl-1,3-diaza-8-azoniaspiro[4.5]decane trifluoroacetate Example 31

A solution of (5R,7S)-1-(3-fluorophenyl)-8-(3-isopropoxybenzyl)-7-methyl-1,3,8-triazaspiro[4.5]decane-2,4-dione (Example 16, 0.043 g, 0.10 mmol), phenylboronic acid (0.048 g, 0.40 mmol), and copper(II)acetate (0.004 g, 0.020 mmol) in MeOH (1 mL) was prepared. After heating at 70° C. for 16 hours, it was cooled to room temperature, and then diluted with MeOH (2 mL). QuadraPure TU resin was added to the reaction mixture. After stirring for 30 minutes at room temperature, the reaction mixture was filtered, and the filtrate was concentrated. The crude material was partitioned between EtOAc (two times) and aqueous NH₄Cl. The EtOAc extract was concentrated under reduced pressure, and the crude material was purified by reverse phase preparative HPLC to afford the desired compound as a TFA salt.

¹H NMR (400 MHz, CD₃OD): δ 7.41-7.52 (m, 5H), 7.25-7.37 (m, 4H), 7.13-7.17 (m, 1H), 7.04 (d, J=7.79 Hz, 1H), 6.77 (s, 2H), 4.58-4.61 (m, 1H), 4.25-4.34 (m, 2H), 3.47 (m, 1H), 2.92 (m, 1H), 2.76-2.79 (m, 1H), 2.65 (m, 1H), 2.40-2.54 (m, 4H), 1.59 (d, J=5.86 Hz, 3H), 1.34 (d, J=4.76 Hz, 6H).

LRMS (M+1)=502.25

The following examples were prepared using a procedure similar to that described for Example 31. In some instances, the compound was isolated as the TFA salt after chromatography, and in some cases the compound was then isolated as the free base by extraction from a suitable aqueous base like bicarbonate solution with a suitable organic solvent like methylene chloride. The free base could then be transformed to the hydrochloride salt by treatment with an ether solution of HCl.

| | | | Mass Spec |
|---|---|---|---|
| Ex # | Structure | Chemical Name | (M + H)⁺ |
| 31-1 | 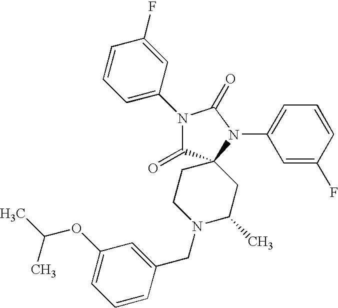 | (5R,7S)-1,3-bis(3- fluorophenyl)-8-(3- isopropoxybenzyl)-7- methyl-1,3,8- triazaspiro[4.5]decane- 2,4-dione | 520.2 |
| 31-2 | 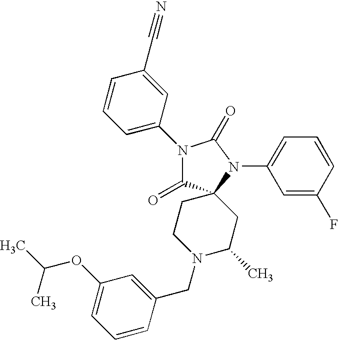 | 3-[(5R,7S)-1-(3- fluorophenyl)-8-(3- isopropoxybenzyl)-7- methyl-2,4-dioxo-1,3,8- triazaspiro[4.5]dec-3- yl]benzonitrile | 527.3 |
| 31-3 | 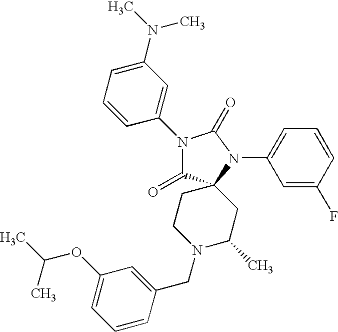 | (5R,7S)-3-[3- (dimethylamino)phenyl]- 1-(3-fluorophenyl)-8-(3- isopropoxybenzyl)-7- methyl-1,3,8- triazaspiro[4.5]decane- 2,4-dione | 545.3 |
| 31-4 | 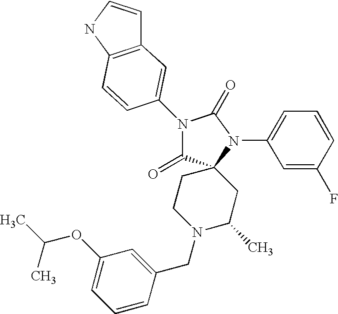 | (5R,7S)-1-(3- fluorophenyl)-3-(1H- indol-5-yl)-8-(3- isopropoxybenzyl)-7- methyl-1,3,8- triazaspiro[4.5]decane- 2,4-dione | 541.3 |
| 31-5 | 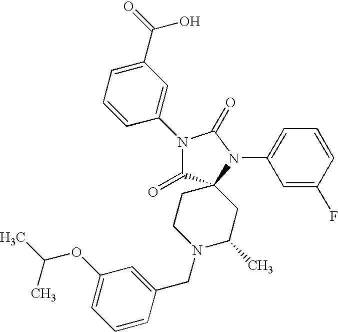 | 3-[(5R,7S)-1-(3- fluorophenyl)-8-(3- isopropoxybenzyl)-7- methyl-2,4-dioxo-1,3,8- triazaspiro[4.5]dec-3- yl]benzoic acid | 546.2 |

### Examples 32

### (5R,7S)-1-(3-fluorophenyl)-8-(3-furylmethyl)-7-methyl-3-[(5-methylisoxazol-3-yl)methyl]-2,4-dioxo-1,3-diaza-8-azoniaspiro[4.5]decane trifluoroacetate

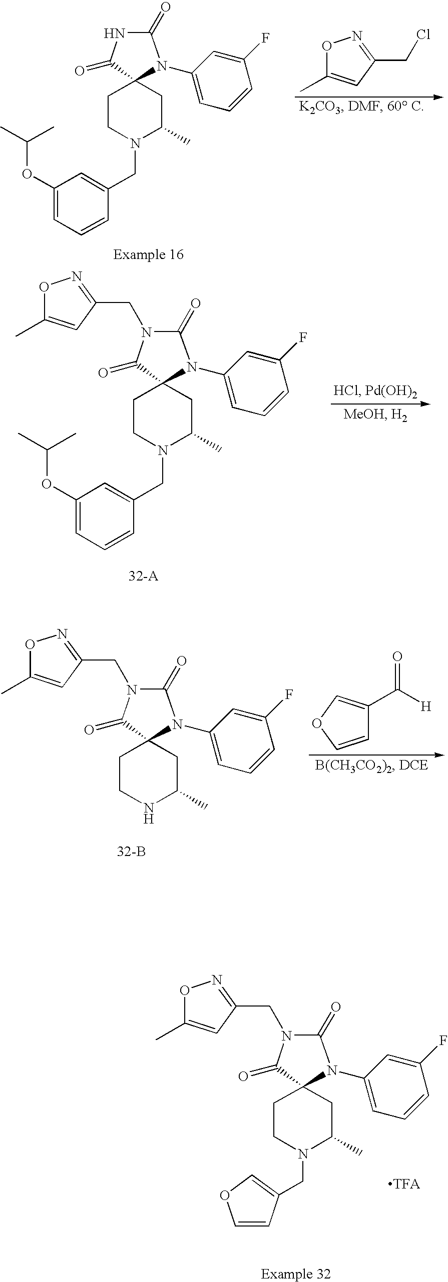

### Step 1. (5R,7S)-1-(3-fluorophenyl)-8-(3-isopropoxybenzyl)-7-methyl-3-[(5-methylisoxazol-3-yl)methyl]-1,3,8-triazaspiro[4.5]decane-2,4-dione 32-A

To a mixture of 3-(chloromethyl)-5-methylisoxazole (1.12 g, 8.53 mmol) and anhydrous K₂CO₃ (3.22 g, 23.27 mmol) was added a solution of (5R,7S)-1-(3-fluorophenyl)-8-(3-isopropoxybenzyl)-7-methyl-1,3,8-triazaspiro[4.5]decane-2,4-dione (Example 16, 3.3. g, 7.76 mmol) in 15 mL DMF. After stirring at 60° C. for 16 hours, the reaction was cooled and filtered and concentrated under reduced pressure. The crude material was chromatographed on a silica gel cartridge using a gradient of DCM/MeOH 100:0 to 50:50. The resulting fractions were concentrated under reduced pressure to a foam which was placed under high vacuum for 16 hrs.

LRMS (M+1)=521.1

### Step 2. (5S,7S)-1-(3-fluorophenyl)-7-methyl-3-[(5-methylisoxazol-3-yl)methyl]-1,3,8-triazaspiro[4.5]decane-2,4-dione 32-B

To (5R,7S)-1-(3-fluorophenyl)-8-(3-isopropoxybenzyl)-7-methyl-3-[(5-methylisoxazol-3-yl)methyl]-1,3,8-triazaspiro[4.5]decane-2,4-dione 32-A (1.4 g, 2.69 mmol) in a round bottom flask was added 1.25 M HCl in MeOH (30 ml). The solution was swirled and the solvent was removed under reduced pressure to leave the HCl salt of 32-1. To this HCl salt in MeOH (30 mL), under nitrogen, was added palladium hydroxide 20 wt on carbon (0.38 g, 0.27 mmol). The flask was fitted with a three way valve. The flask was evacuated with high vacuum until the solvent bubbled then nitrogen was flushed in. This was repeated. The flask was evacuated a third time and hydrogen was introduced by way of a filled balloon on the three-way valve. The flask was evacuated again and the hydrogen flushed in and the balloon was allowed to remain in an open position on the valve. After stirring at room temperature for 16 hours, the reaction was evacuated and flushed with nitrogen three times successively. The nitrogen filled flask was opened and the contents were vacuum filtered through MeOH wetted Celite™ maintaining a brisk flush of nitrogen over the funnel and the bed was rinsed with MeOH (100 ml) never allowing the bed to dry. The filtrate was concentrated under reduced pressure and then evaporated twice from toluene to give the HCl salt of compound 32-2. This salt was partitioned between DCM and saturated sodium bicarbonate solution. The organics were dried over MgSO₄, filtered and concentrated under reduced pressure to give the free base of 32-2 which was used without further purification.

LRMS (M+1)=373.0

### Step 3. (5R,7S)-1-(3-fluorophenyl)-8-(3-furylmethyl)-7-methyl-3-[(5-methylisoxazol-3-yl)methyl]-2,4-dioxo-1,3-diaza-8-azoniaspiro[4.5]decane trifluoroacetate Example 32

To a solution of (5S,7S)-1-(3-fluorophenyl)-7-methyl-3-[(5-methylisoxazol-3-yl)methyl]-1,3,8-triazaspiro[4.5]decane-2,4-dione 32-B (0.1 g, 0.27 mmol) in DCE (2 ml) was added 3-furaldehyde (0.026 g, 0.27 mmol) followed by triacetoxyborohydride resin 2.17 gm/mmol (0.37 g, 0.8 mmol). The mixture was stirred vigorously under nitrogen for 16 hrs. The reaction was filtered through a glass fiber filter and the filtrate was rinsed with DCE and the filtrate was concentrated under reduced pressure. The residue was dissolved in 2 ml of DMSO and purified by preparative reverse phase HPLC. The desired fractions were concentrated under reduced pressure to give Example 32 as a solid.

LRMS (M+1)=453.2

¹H NMR (400 MHz, CD₃OD): δ 7.57 (s, 2H), 7.42 (dd, J=6.8 Hz, 1H), 7.25 (dd, J=1.8, 9.2 Hz, 1H), 7.22 (d, J=7.9 Hz, 2H), 6.27 (s, 1H), 6.16 (s, 1H), 4.78 (s, 2H), 4.37 (d, J=14.6 Hz, 1H), 4.11 (d, J=14.3 Hz, 1H), 3.38 (d, J=10.4 Hz, 1H), 2.78 (bs, 1H), 2.55 (m, 4H), 2.41 (s, 3H), 2.30 (m, 1H), 1.46 (d, J=5.9 Hz, 3H).

The following examples were prepared using a procedure similar to that described for Example 32. In some instances, the compound was isolated as the TFA salt after chromatography, and in some cases the compound was then isolated as the free base by extraction from a suitable aqueous base like bicarbonate solution with a suitable organic solvent like methylene chloride. The free base could then be transformed to the hydrochloride salt by treatment with an ether solution of HCl.

| | | | Mass Spec |
|---|---|---|---|
| Ex # | Structures | Chemical Names | (M + H)⁺ |
| 32-1 | 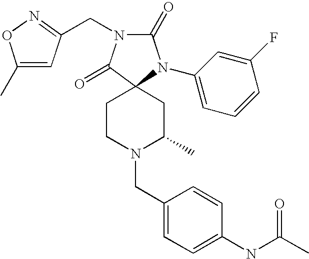 | N-[4-({(5R,7S)-1-(3- fluorophenyl)-7-methyl-3- [(5-methylisoxazol-3- yl)methyl]-2,4-dioxo- 1,3,8-triazaspiro[4.5]dec- 8- yl}methyl)phenyl]acetamide | 520.1 |
| 32-2 | 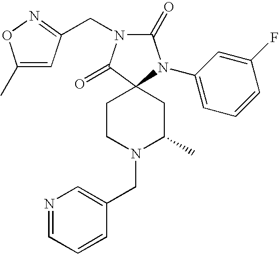 | (5R,7S)-1-(3- fluorophenyl)-7-methyl-3- [(5-methylisoxazol-3- yl)methyl]-8-(pyridin-3- ylmethyl)-1,3,8- triazaspiro[4.5]decane-2,4- dione | 464.1 |
| 32-3 | 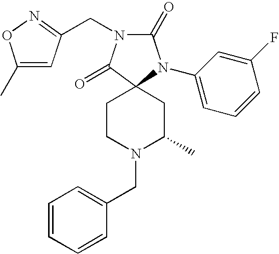 | (5R,7S)-8-benzyl-1-(3- fluorophenyl)-7-methyl-3- [(5-methylisoxazol-3- yl)methyl]-1,3,8- triazaspiro[4.5]decane-2,4- dione | 463.0 |
| 32-4 | 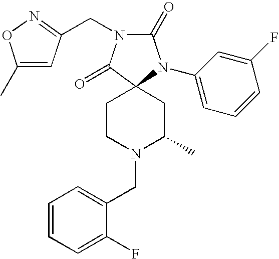 | (5R,7S)-8-(2- fluorobenzyl)-1-(3- fluorophenyl)-7-methyl-3- [(5-methylisoxazol-3- yl)methyl]-1,3,8- triazaspiro[4.5]decane-2,4- dione | 481.0 |
| 32-5 | 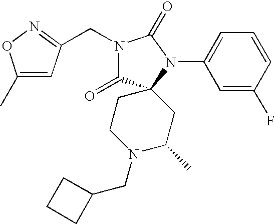 | (5R,7S)-8- (cyclobutylmethyl)-1-(3- fluorophenyl)-7-methyl-3- [(5-methylisoxazol-3- yl)methyl]-1,3,8- triazaspiro[4.5]decane-2,4- dione | 441.0 |

### Example 33

### 5R,7S)-8-benzyl-3-{[5-(3,4-dichlorophenyl)isoxazol-3-yl]methyl}-1-(3-fluorophenyl)-7-methyl-1,3,8-triazaspiro[4.5]decane-2,4-dione

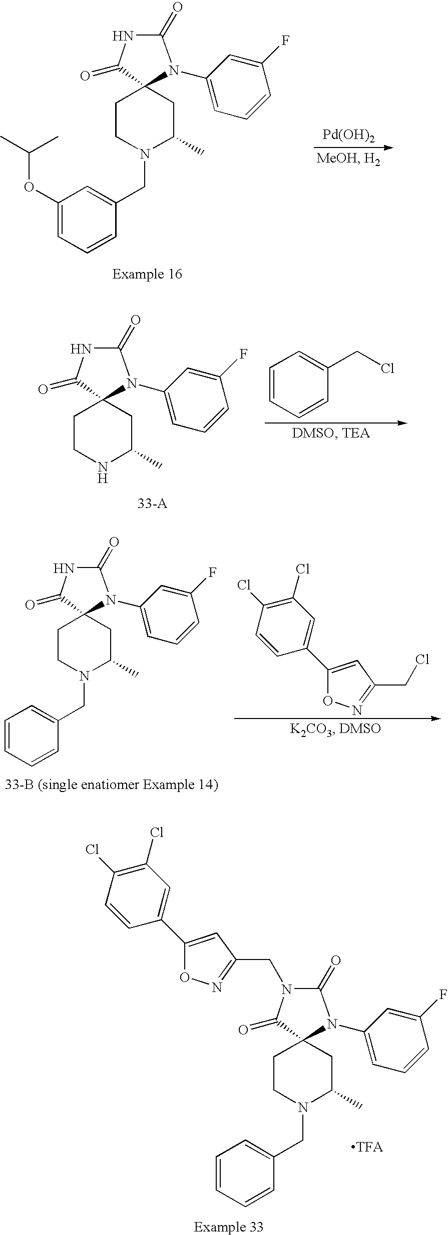

### Step 1. (5R,7S)-1-(3-fluorophenyl)-7-methyl-1,3,8-triazaspiro[4.5]decane-2,4-dione 33-A

To a solution of (7S)-1-(3-fluorophenyl)-8-(3-isopropoxybenzyl)-7-methyl-1,3,8-triazaspiro[4.5]decane-2,4-dione (Example 16, (5.0 g, 11.75 mmol) in MeOH (75 ml) under nitrogen was added palladium hydroxide 20 wt % on carbon (0.825 g, 1.75 mmol) and the flask was fitted with a three-way valve. The flask was evacuated with high vacuum until the solvent bubbled then nitrogen was flushed in. This was repeated. The flask was evacuated a third time and hydrogen was introduced by way of a filled balloon on the three-way valve. The flask was evacuated again and the hydrogen flushed in and the balloon was allowed to remain in an open position on the valve. After stirring at room temperature for 16 hours, the reaction was evacuated and flushed with nitrogen three times successively. The nitrogen filled flask was opened and the contents were vacuum filtered through methanol wetted Celite™ maintaining a brisk flush of nitrogen over the funnel and the bed was rinsed with MeOH (100 ml), never allowing the bed to dry. The filtrate was concentrated under reduced pressure and then evaporated twice from toluene to give the desired product as a solid.

LRMS (M+1)=277.9

### Step 2. (5R,7S)-8-benzyl-1-(3-fluorophenyl)-7-methyl-1,3,8-triazaspiro[4.5]decane-2,4-dione 33-B (Alternative synthesis of Example 14, single 5R,7S enantiomer)

To a solution of (5R,7S)-1-(3-fluorophenyl)-7-methyl-1,3,8-triazaspiro[4.5]decane-2,4-dione 33-A (0.1 g, 0.361 mmol) in DMSO (2 ml) was added benzyl chloride (0.046 g, 0.505 mmol) and triethylamine (0.101 ml, 0.72 mmol) and the reaction was allowed to stir at room temperature under nitrogen for 16 h. The reaction was filtered through a glass fiber filter and rinsed with methanol. The resulting 2.5 ml filtrate was injected into the preparative reverse phase HPLC and the desired product was eluted off using a water (0.1% TFA)/acetonitrile (0.1% TFA) gradient 95:5 to 5:95. The desired fractions were concentrated under reduced pressure to give the desired product as a solid. The residue was dissolved in DCM (5 ml) and partitioned with saturated sodium bicarbonate solution. The organic was separated and the aqueous was washed with more DCM (2 ml). The combined organics were dried over Na₂SO₄, filtered and concentrated to give the desired product as the free base.

LRMS (M+1)=367.9

### Step 3. (5R,7S)-8-benzyl-3-{[5-(3,4-dichlorophenyl)isoxazol-3-yl]methyl}-1-(3-fluorophenyl)-7-methyl-1,3,8-triazaspiro[4.5]decane-2,4-dione trifluoroacetate Example 33

To a solution of (5R,7S)-8-benzyl-1-(3-fluorophenyl)-7-methyl-1,3,8-triazaspiro[4.5]decane-2,4-dione 33-B (0.037 g, 0.1 mmol) in DMSO (1 ml) under nitrogen was added 3-(chloromethyl)-5-(3,4-dichlorophenyl)isoxazole followed by K₂CO₃ (0.069 g,0.5 mmol) and the reaction was stirred under nitrogen for 16 hrs. The reaction was filtered through a glass fiber filter and rinsed with MeOH. The resulting 2.5 ml filtrate was injected into the preparative reverse phase HPLC and the desired product was eluted off using a water (0.1% TFA)/acetonitrile (0.1% TFA) gradient 95:5 to 5:95. The desired fractions were concentrated under reduced pressure to give the desired product as a solid.

LRMS (M+1)=594.4

¹H NMR (400 MHz, CD₃OD): δ 8.01 (d, J=1.8 Hz, 1H), 7.76 (m, 1H), 7.68 (m, 1H), 7.50 (m, 1H), 7.41 (m, 2H), 7.33 (m, 1H), 7.22 (m, 4H), 7.13 (m, 1H), 6.90 (s, 1H), 4.87 (m, 2H), 4.33 (bs, 2H), 3.42 (m, 1H), 2.85 (m, 1H), 2.66 (d, J=10.6 Hz, 1H), 2.48 (m, 4H), 1.58 (d, J=5.3 Hz, 3H).

The following examples were prepared using a procedure similar to that described for Example 33. In some instances, the compound was isolated as the TFA salt after chromatography, and in some cases the compound was then isolated as the free base by extraction from a suitable aqueous base like bicarbonate solution with a suitable organic solvent like methylene chloride. The free base could then be transformed to the hydrochloride salt by treatment with an ether solution of HCl.

| | | | Mass Spec |
|---|---|---|---|
| Ex # | Structure | Chemical Name | (M + H)⁺ |
| 33-1 | 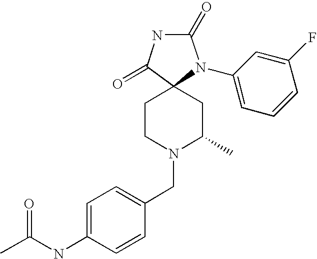 | N-(4-{[(5R,7S)-1-(3- fluorophenyl)-7-methyl-2,4- dioxo-1,3,8- triazaspiro[4.5]dec-8- yl]methyl}phenyl)acetamide | 425.0 |
| 33-2 | 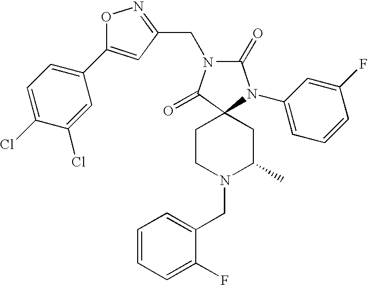 | (5R,7S)-3-{[5-(3,4- dichlorophenyl)isoxazol-3- yl]methyl}-8-(2- fluorobenzyl)-1-(3- fluorophenyl)-7-methyl- 1,3,8- triazaspiro[4.5]decane-2,4- dione | 613.2 |
| 33-3 | 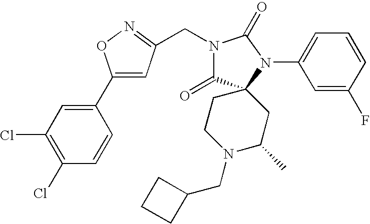 | (5R,7S)-8- (cyclobutylmethyl)-3-{[5-(3,4- dichlorophenyl)isoxazol-3- yl]methyl}-1-(3- fluorophenyl)-7-methyl- 1,3,8- triazaspiro[4.5]decane-2,4- dione | 571.0 |

### Examples 34-1 and 34-2

### (5R,7S)-1-(3-cyanophenyl)-8-(3-isopropoxybenzyl)-7-methyl-2,4-dioxo-1,3-diaza-8-azoniaspiro[4.5]decane trifluoroacetate (Example 34.1) and (5R,7S)-1-(3-cyanophenyl)-8-(3-isopropoxybenzyl)-7-methyl-3-[(5-methylisoxazol-3-yl)methyl]-2,4-dioxo-1,3-diaza-8-azoniaspiro[4.5]decane trifluoroacetate (Example 34.2)

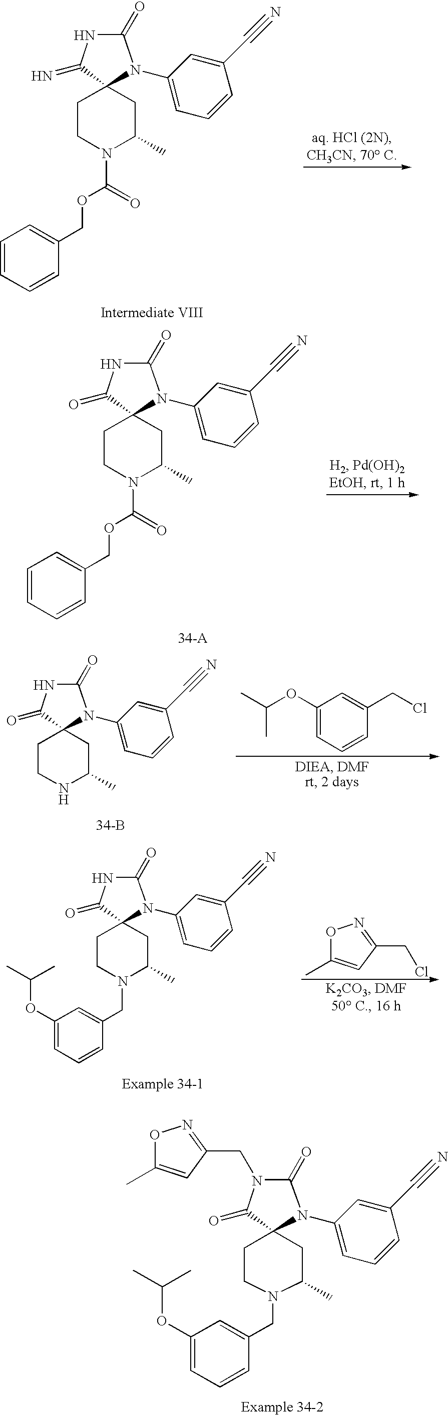

### Step 1: Benzyl (5R,7S)-1-(3-cyanophenyl)-7-methyl-2,4-dioxo-1,3,8-triazaspiro[4.5]decane-8-carboxylate Intermediate 34-A

The benzyl (4Z,5R,7S)-1-(3-cyanophenyl)-4-imino-7-methyl-2-oxo-1,3,8-triazaspiro[4.5]decane-8-carboxylate (Intermediate VIII) was prepared in a manner similar to that described for example 11-6 in WO2007/011833 by using 3-cyanoaniline and condition A in that example.

LCMS (M+1)=418.11

To the solution of benzyl (4Z,5R,7S)-1-(3-cyanophenyl)-4-imino-7-methyl-2-oxo-1,3,8-triazaspiro[4.5]decane-8-carboxylate (intermediate VIII, 0.42 g, 1.01 mmol) in CH₃CN (2.5 mL) was added aqueous HCl (2 N, 2.5 mL) in a 48 mL sealed tube. The reaction mixture was purged with nitrogen, sealed and heated at 70° C. for 2 hours then 60° C. for 16 hours. The reaction mixture was concentrated under reduced pressure to give the desired product.

LRMS (M+1)=418.99

### Step 2: 3-[(5R,7S)-7-methyl-2,4-dioxo-1,3,8-triazaspiro[4.5]dec-1-yl]benzonitrile 34-B

To the solution of benzyl (5R,7S)-1-(3-cyanophenyl)-7-methyl-2,4-dioxo-1,3,8-triazaspiro[4.5]decane-8-carboxylate 34-1 (0.050 g, 0.12 mmol) in EtOH (6 mL) under nitrogen was added Pearlman's catalyst (Pd(OH)₂, 20 wt % on C, 0.010 g) in a 50 mL round bottom flask. Then standard hydrogenation conditions (similar to that described in example 33) using a hydrogen filled balloon was performed at room temperature for 1 hour. The mixture was filtered through celite, and the residue was washed with MeOH (two times). The organic filtrate was concentrated under reduced pressure to give the desired product.

LRMS (M+1)=285.10

### Step 3: (5R,7S)-1-(3-cyanophenyl)-8-(3-isopropoxybenzyl)-7-methyl-2,4-dioxo-1,3-diaza-8-azoniaspiro[4.5]decane trifluoroacetate Example 34-1

The hydrogenation product 3-[(5R,7S)-7-methyl-2,4-dioxo-1,3,8-triazaspiro[4.5]dec-1-yl]benzonitrile 34-B (0.15 g, 0.529 mmol) and 1-(chloromethyl)-3-isopropoxybenzene (prepared as described in patent WO2007/011833, example 11, step 10c, 0.196 g, 1.06 mmol) were dissolved in anhydrous DMF (0.8 mL), and DIEA was added to the above solution to adjust the pH to 10. The reaction was stirred at room temperature for 2 days, and purified by reverse phase preparative HPLC to give the desired product as a TFA salt.

LRMS (M+1)=433.0

### Step 4: (5R,7S)-1-(3-cyanophenyl)-8-(3-isopropoxybenzyl)-7-methyl-3-[(5-methylisoxazol-3-yl)methyl]-2,4-dioxo-1,3-diaza-8-azoniaspiro[4.5]decane trifluoroacetate Example 34-2

To a mixture of 3-(chloromethyl)-5-methylisoxazole (0.0217 g, 0.165 mmol) and anhydrous K₂CO₃ (0.138 g, 0.247 mmol) was added a solution of 3-[(5R,7S)-8-(3-isopropoxybenzyl)-7-methyl-2,4-dioxo-1,3,8-triazaspiro[4.5]dec-1-yl]benzonitrile trifluoroacetate Example 34-1 (0.045 g, 0.082 mmol) in anhydrous DMF (1 mL). After stirring at 50° C. for 16 hours, the reaction was quenched with aqueous NH₄Cl and extracted with EtOAc (two times). The EtOAc extract was concentrated under reduced pressure. Purification by reverse phase preparative HPLC afforded the desired product as a TFA salt.

¹H NMR (500 MHz, CD₃OD): δ 7.83 (m, 1H), 7.73 (m, 1H), 7.68-7.69 (m, 1H), 7.51 (m, 1H), 7.31-7.35 (m, 1H), 7.06-7.07 (m, 1H), 6.73-6.76 (m, 2H), 6.16 (s, 1H), 4.78 (s, 2H), 4.62-4.65 (m, 1H), 4.34 (d, J=13.43 Hz, 1H), 4.23 (d, J=13.67 Hz, 1H), 3.44 (s, 1H), 2.80 (m, 1H), 2.65 (m, 1H), 2.26-2.54 (m, 4H), 2.41 (s, 3H), 1.58 (d, J=5.01 Hz, 3H), 1.34 (m, 6H).

LRMS (M+1)=528.21

The following examples were prepared using a procedure similar to that described for Example 34. In some instances, the compound was isolated as the TFA salt after chromatography, and in some cases the compound was then isolated as the free base by extraction from a suitable aqueous base like bicarbonate solution with a suitable organic solvent like methylene chloride. The free base could then be transformed to the hydrochloride salt by treatment with an ether solution of HCl.

| | | | Mass Spec |
|---|---|---|---|
| Ex # | Structure | Chemical Name | (M + H)⁺ |
| 34-3 | 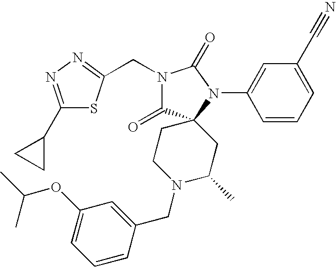 | 3-[(5R,7S)-3-[(5- cyclopropyl-1,3,4- thiadiazol-2-yl)methyl]- 8-(3-isopropoxybenzyl)- 7-methyl-2,4-dioxo- 1,3,8-triazaspiro[4.5]dec- 1-yl]benzonitrile | 571.2 |
| 34-4 | 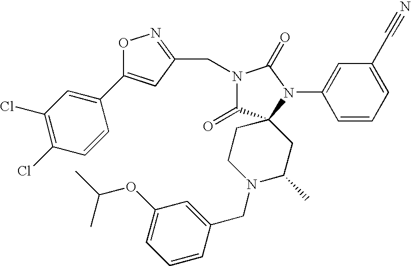 | 3-[(5R,7S)-3-{[5-(3,4- dichlorophenyl)isoxazol- 3-yl]methyl}-8-(3- isopropoxybenzyl)-7- methyl-2,4-dioxo-1,3,8- triazaspiro[4.5]dec-1- yl]benzonitrile | 658.2 |
| 34-5 | 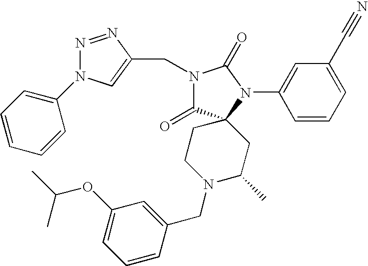 | 3-{(5R,7S)-8-(3- isopropoxybenzyl)-7- methyl-2,4-dioxo-3-[(1- phenyl-1H-1,2,3-triazol- 4-yl)methyl]-1,3,8- triazaspiro[4.5]dec-1- yl}benzonitrile | 590.3 |
| 34-6 | 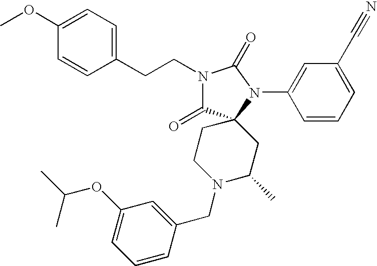 | 3-{(5R,7S)-8-(3- isopropoxybenzyl)-3-[2- (4- methoxyphenyl)ethyl]-7- methyl-2,4-dioxo-1,3,8- triazaspiro[4.5]dec-1- yl}benzonitrile | 567.3 |
| 34-7 | 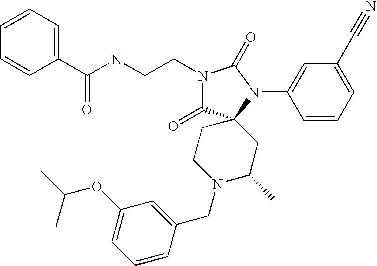 | N-{2-R5R,7S)-1-(3- cyanophenyl)-8-(3- isopropoxybenzyl)-7- methyl-2,4-dioxo-1,3,8- triazaspiro[4.5]dec-3- yl]ethyl}benzamide | 580.3 |
| 34-8 | 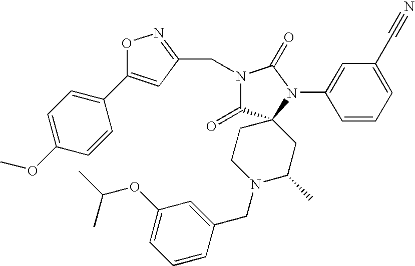 | 3-((5R,7S)-8-(3- isopropoxybenzyl)-3- {[5-(4- methoxyphenyl)isoxazol- 3-yl]methyl}-7-methyl- 2,4-dioxo-1,3,8- triazaspiro[4.5]dec-1- yl)benzonitrile | 620.2 |

### Example 35

### (5R,7S)-1-(3-fluorophenyl)-7-methyl-8-[(2′-methylbiphenyl-3-yl)methyl]-3-[(5-methylisoxazol-3-yl)methyl]-1,3,8-triazaspiro[4.5]decane-2,4-dione

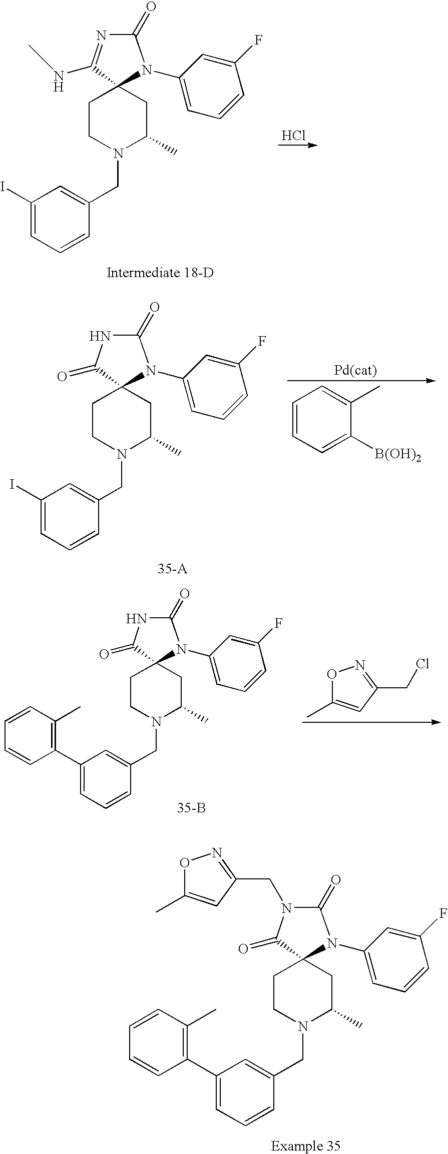

### Step 1. (5R,7S)-1-(3-fluorophenyl)-8-(3-iodobenzyl)-7-methyl-1,3,8-triazaspiro[4.5]decane-2,4-dione 35-A

A solution of 1.55 g (3.097 mmol) Intermediate 18-D in 3 ml 1 N HCl was stirred at 60 degrees C. overnight. The solution was made basic with 6 N NaOH and extracted with dichloromethane. The organic layer was dried over sodium sulfate, filtered and evaporated. The residue was purified on silica eluting with a gradient of 0-10% MeOH in dichloromethane to give the product.

LRMS (M+1)=493.6

### Step 2. (5R,7S)-1-(3-fluorophenyl)-7-methyl-8-[(2′-methylbiphenyl-3-yl)methyl]-1,3,8-triazaspiro[4.5]decane-2,4-dione 35-B

(5R,7)-1-(3-fluorophenyl)-8-(3-iodobenzyl)-7-methyl-1,3,8-triazaspiro[4.5]decane-2,4-dione (Intermediate 35-A, (950 mg, 1.92 mmole), palladium catalyst PdCl₂Fe—CH₂Cl₂ (79 mg, 0.096 mmole), and cesium carbonate (1.88 g, 5.78 mmole) and 2-tolylboronic acid (314 mg, 2.31 mmole) were measured into a 20 ml microwave vial. The vial was capped and flushed with nitrogen. A degassed mixture of 1:1 THF/water (6 mL) was added via syringe and the reaction was heated to 120 degrees C. in the microwave for 5 minutes. The solution was transferred to a separatory funnel, diluted with water and extracted with ethyl acetate and dichloromethane. The organic layer was dried over sodium sulfate, filtered and evaporated. The residue was purified on silica eluting with a gradient of 0-100% ethyl acetate/hexanes to give the product.

LRMS (M+1)=457.9

### Step 3. (5R,7S)-1-(3-fluorophenyl)-7-methyl-8-[(2′-methylbiphenyl-3-yl)methyl]-3-[(5-methylisoxazol-3-yl)methyl]-1,3,8-triazaspiro[4.5]decane-2,4-dione Example 35

In a manner similar to that described for Example 32, (5R,7S)-1-(3-fluorophenyl)-7-methyl-8-[(2′-methylbiphenyl-3-yl)methyl]-1,3,8-triazaspiro[4.5]decane-2,4-dione was treated with 3-(chloromethyl)-5-methylisoxazole and potassium carbonate in DMF at room temperature to give the desired product.

LRMS (M+1)=553.0

The product was treated with 2 M HCl in ether to give the hydrochloride salt.

HRMS (M+1)=553.2643 (measured), 553.2610 (calculated).

The following abbreviations are used throughout the text:

Me: methyl

Et: ethyl

t-Bu: tert-butyl

Ar: aryl

Ph: phenyl

Bn: benzyl

Ac: acetyl

TMSCN: trimethylsilyl cyanide

DMSO: dimethylsulfoxide

EDTA: ethylene diamine tetraacetic acid

Boc: tert-butyloxy carbonyl

CHAPS: 3-[(3-cholamidopropyl)dimethylammonio]-2-hydroxy-1-propanesulfonate

BSA: bovine serum albumin

TFA: trifluoracetic acid

DME: dimethoxyethane

DPPA: diphenylphosphorlyazide

DCE: dichloroethane

DCM: dichloromethane

THF: tetrahydrofuran

BOP: benzotriazolyl-N-oxy-tris(dimethylamino)phosphonium hexaflurophosphate

DMF: dimethylformamide

DIBAL: diisobutylaluminum hydride

h: hour

min: minutes

rt: room temperature

aq: aqueous

HPLC: high performance liquid chromatography

MS: mass spectrometry

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1. A compound of formula (I): 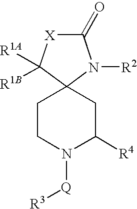
wherein
X is selected from the group consisting of
(1) N—R⁵,
(2) O, and
(3) S,
and R⁵ is selected from the group consisting of
(a) hydrogen,
(b) —C₁₋₁₀ alkyl,
(c) —C₂₋₁₀ alkenyl,
(d) —C₃₋₁₂ cycloalkyl,
(e) —C₀₋₆ alkyl-aryl, and
(f) —C₀₋₆ alkyl-heteroaryl,
wherein said alkyl, alkenyl, cycloalkyl, aryl and heteroaryl R⁵ moiety is optionally substituted with one or more
(i) aryl,
(ii) heteroaryl,
(iii) halogen,
(iv) —C₁₋₁₀ alkyl,
(v) —C₁₋₁₀ alkyl,
(vi) —C₃₋₁₂ cycloalkyl,
(vii) —NC(═O)—R⁶,
(viii) —C(═O)NR⁶R⁶′,
(ix) —C(═O)—OR⁶,
(x) —C(═O)—R⁶,
(xi) —CN
(xii) —NR⁶R⁶′,
wherein said aryl, alkly, cycloalkyl and heteroaryl moiety is optionally substituted with one or more
(I) halogen,
(II) —C₁₋₆ alkyl,
(III) —OC₁₋₆ alkyl,
R^{1A} and R^{1B} are each hydrogen, provided that when X is NR⁵, then R^{1A} and R^{1B} may together form ═O;
R² is selected from the group consisting of
(1) hydrogen,
(2) —C₁₋₁₀ alkyl,
(3) —C₂₋₁₀ alkenyl,
(4) —C₂₋₁₀ alkynyl,
(5) —C₃₋₁₂ cycloalkyl,
(6) a heterocyclic group having 4 to 8 ring atoms, wherein one ring atom is a heteroatom selected from the group consisting of nitrogen and oxygen,
(7) aryl, and
(8) heteroaryl,
wherein said alkyl, cycloalkyl, heterocyclic group, alkenyl, alkynyl, aryl or heteroaryl R² moiety is optionally substituted with one or more
(a) halo,
(b) —OH,
(c) —CN,
(d) —C₁₋₁₀ alkyl,
(e) —C₂₋₁₀ alkenyl,
(f) —C₂₋₁₀ alkynyl,
(g) —C₃₋₁₂ cycloalkyl,
(h) —O—C₁₋₁₀ alkyl,
(i) —C₀₋₆ alkyl-aryl, or
(j) —C₀₋₆ alkyl-heteroaryl,
wherein said alkyl, alkenyl, alkynyl, aryl and heteroaryl moiety is optionally substituted with one or more
(i) halo,
(ii) —OH,
(iii) —CN,
(iv) —C₁₋₆ alkyl,
(v) —C₂₋₆ alkenyl,
(vi) —OC₁₋₆ alkyl,
(vii) —C₁₋₆ haloalkyl,
(viii) —SO₂C₁₋₃ alkyl,
(ix) —SO₂NR⁶R⁶′,
(x) —OC₂R⁶,
(xi) —NR⁶SO₂R⁶′,
(xii) —CONR⁶R⁶′;
(xiii) —NC(═O)—C₀₋₃ alkyl-NR⁶R⁶′;
(xiv) —NC(═O)R⁶
(xv) —NR⁶R⁶′, and
(xvi) a heterocyclic group having 4 to 8 ring atoms, wherein one ring atom
is a heteroatom selected from the group consisting of nitrogen and oxygen;
Q is —C₁₋₆ alkylene, wherein said alkylene is optionally substituted with one or more:
(a) halo,
(b)
(c) —CN,
(d) —C₁₋₁₀ alkyl
(e) —C₃₋₁₂ cycloalkyl,
(f) —O—C₁₋₁₀ alkyl,
(g) aryl, and
(h) heteroaryl;
R³ is selected from the group consisting of
(1) hydrogen,
(2) —C₁₋₁₀alkyl,
(3) —C₂₋₁₀ alkenyl,
(4) —C₂₋₁₀ alkynyl,
(5) —C₃₋₁₂ cycloalkyl,,
(6) —C₃₋₁₂ cycloalkenyl,
(7) aryl, and
(8) heteroaryl,
wherein said alkyl, cycloalkyl, cycloalkenyl, alkenyl, alkynyl or aryl or heteroaryl R³ moiety is optionally substituted with one or more
(a) halo,
(b) —OH,
(c) —CN,
(d) —C₁₋₁₀alkyl,
(e) —C₂₋₁₀ alkenyl,
(f) —C₃₋₁₂ cycloalkyl,
(g) —O—C₃₋₁₂ cycloalkyl
(h) —O—C₁₋₁₀ alkyl,
(i) —O—C₃₋₁₂ heterocyclic, wherein said heterocyclic group has from 4 to 8 ring atoms, wherein one ring atom is a heteroatom selected from the group consisting of nitrogen, sulfur and oxygen,
(j) aryl,
(k) heteroaryl,
(l) —NR⁶R⁶′,
and said alkyl, cycloalkyl, aryl and heteroaryl moiety is optionally substituted with one or more
(i) halo,
(ii) —OH,
(iii) —CN,
(iv) —C₁₋₁₀ alkyl,
(v) —OC₁₋₁₀ alkyl, and
(vi) —NR⁶R⁶′
(vii) —C₂₋₆ alkenyl,
(viii) —C₁₋₆ haloalkyl,
(ix) —SO₂C₁₋₃ alkyl,
(x) —SO₂NR⁶R⁶′,
(xi) —CONR⁶R⁶,
(xii) —NR⁵COR⁵′, wherein R⁵′ is selected from the same group as R⁵, or
(xiii) —NR⁷SO₂R⁶, wherein R⁷ is selected from the group consisting of
(A) hydrogen
(B) —C₁₋₁₀ alkyl, and
(C) —C₃₋₄ alkenyl;
R⁴ is selected from the group consisting of
(1) hydrogen,
(2) —C₁₋₁₀ alkyl, and
(3) —C₃₋₄ alkenyl,
wherein said alkyl or alkenyl R⁴ group is optionally substituted with one or more
(a) halo,
(b) —OH
(c) —C₁₋₆ alkyl,
(d) —CN,
(e) —O—C₁₋₁₀ alkyl,
(f) —NR⁸R⁹, wherein R⁸ and R⁹ are selected from the group consisting of
(i) hydrogen, and
(ii) —C₁₋₆ alkyl,
(g) —S(O)ₙ—C₁₋₆ alkyl, wherein n is 0, 1 or 2,
(h) —C(═O)—R⁷, wherein R⁷ is selected from the group consisting of
(i) hydrogen,
(ii) —OH,
(iii) —C₁₋₆ alkyl, and
(iv) —OC₁₋₆ alkyl, and
(v) aryl;
R⁶ and R⁶′ are selected from the group consisting of
(1) hydrogen,
(2) —C₁₋₆ alkyl,
(3) —C₃₋₇ cycloalkyl,
(4) —C₁₋₆ haloalkyl,
(5) —C₀₋₆ alkyl-aryl,
(6) —C₀₋₆ alkyl-heteroaryl,
(7) halo, and
(8) a heterocyclic group having 4 to 8 ring atoms, wherein one ring atom is a heteroatom selected from the group consisting of nitrogen and oxygen, wherein said aryl or heteroaryl R⁵ moiety is optionally substituted with one or more
(a) halo,
(b) —C₁₋₆ alkyl,
(c) O—C₁₋₆ alkyl, and
(d) —NO₂;
and pharmaceutically acceptable salts thereof, and individual enantiomers and diastereomers thereof.

2. A compound of claim 1, wherein X is NR⁵.

3. A compound of claim 1, wherein R² is phenyl, wherein the phenyl is optionally substituted with one or more
(i) halo,
(ii) —OH,
(iii) —CN,
(iv) —C₁₋₁₀ alkyl, and
(v) phenyl optionally substituted with
(A) halo,
(B) —OH,
(C) —CN,
(D) —C₁₋₆ alkyl,
(E) —OC₁₋₆ alkyl,
(F) —SO₂C₁₋₃ alkyl,
(G) —SO₂NR⁵R⁵′,
(H) —NR⁵SO₂C₁₋₃alkyl,
(I) —CO₂R⁵, and
(J) —CONR⁵R⁵′.

4. A compound of claim 1, wherein Q is C₁₋₃ alkylene and R³ is phenyl, wherein the phenyl is optionally substituted with one or more
(A) halo,
(B) —OH,
(C) —CN,
(D) —C₁₋₁₀ alkyl,
(E) —OC₁₋₁₀ alkyl, and
(F) phenyl, optionally substituted with
(i) —C₁₋₆ alkyl,
(ii) —OC₁₋₆ alkyl,
(iii) NR⁵R⁵′.

5. A compound of claim 1, wherein R⁴ is —C₁₋₆ alkyl.

6. A compound of claim 1, wherein the compound of formula (I) is a compound of formula (II) 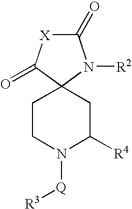
and pharmaceutically acceptable salts thereof, and individual enantiomers and diastereomers thereof.

7. A compound of claim 6, wherein the compound of formula (II is a compound of formula (II″): 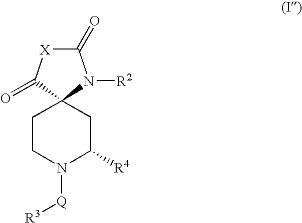
and pharmaceutically acceptable salts thereof, and individual enantiomers and diastereomers thereof.

8. A compound of claim 6, wherein X is NR⁵.

9. A compound of any of claim 6, wherein R² is phenyl, wherein the phenyl is optionally substituted with one or more
(i) halo,
(ii) —OH,
(iii) —CN,
(iv) —C₁₋₁₀alkyl, or
(v) phenyl, optionally substituted with
(A) halo,
(B) —OH,
(C) —CN,
(D) —C₁₋₆ alkyl,
(E) —OC₁₋₆ alkyl,
(F) —SO₂C₁₋₃ alkyl,
(G) —SO₂NR⁵R⁵′,
(H) —NR⁵SO₂C₁₋₃alkyl,
(I) —CO₂R⁵, and
(J) —CONR⁵R⁵′, and
(K) —NR⁵CO₂R⁵′.

10. A compound of claim 6, wherein
R⁴ is C₁₋₆ alkyl.

11. A compound of claim 1, wherein the compound of formula (I) is a compound of formula (III) 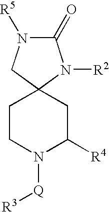
and pharmaceutically acceptable salts thereof, and individual enantiomers and diastereomers thereof.

12. A compound of claim 1, wherein the compound of formula (I) is a compound of formula (IV) 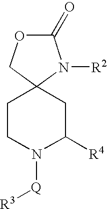
and pharmaceutically acceptable salts thereof, and individual enantiomers and diastereomers thereof.

13. A compound of claim 1, which is selected from the group consisting of
1-(3-fluorophenyl)-8-[(2′-methylbiphenyl-3-yl)methyl]-1,3,8-triazaspiro[4.5]decane-2,4-dione;
4′-fluoro-2′-[8-(3-isopropoxybenzyl)-2,4-dioxo-1,3,8-triazaspiro[4.5]dec-1-yl]-N,N,3-trimethylbiphenyl-4-sulfonamide;
4′-fluoro-2′-[8-(3-isopropoxybenzyl)-2,4-dioxo-1,3,8-triazaspiro[4.5]dec-1-yl]-N,N-dimethylbiphenyl-4-sulfonamide;
4′-fluoro-2′-[8-(3-isopropoxybenzyl)-3-methyl-2,4-dioxo-1,3,8-triazaspiro[4.5]dec-1-yl]-N,N-dimethylbiphenyl-4-sulfonamide;
4′-fluoro-2′-[8-(3-isopropoxybenzyl)-2,4-dioxo-3-propyl-1,3,8-triazaspiro[4.5]dec-1-yl]-N,N-dimethylbiphenyl-4-sulfonamide;
4′-fluoro-2′-[8-(3-isopropoxybenzyl)-2,4-dioxo-1,3,8-triazaspiro[4.5]dec-1-yl]-N,N-dimethylbiphenyl-4-sulfonamide;
2′-(8-benzyl-2,4-dioxo-1,3,8-triazaspiro[4.5]dec-1-yl)-4′-fluoro-N,N-dimethylbiphenyl-4-sulfonamide;
4′-fluoro-2′-[8-(3-isopropoxybenzyl)-2,4-dioxo-1,3,8-triazaspiro[4.5]dec-1-yl]-N,N,3-trimethylbiphenyl-4-carboxamide;
5-{4-fluoro-2-[8-(3-isopropoxybenzyl)-2,4-dioxo-1,3,8-triazaspiro[4.5]dec-1-yl]phenyl}-N,N-dimethylthiophene-2-sulfonamide;
(5R,7S)-8-benzyl-1-(2-bromo-5-fluorophenyl)-7-methyl-1,3,8-triazaspiro[4.5]decane-2,4-dione;
(5R,7S)-1-(2-bromo-5-fluorophenyl)-8-(3-isopropoxybenzyl)-7-methyl-1,3,8-triazaspiro[4.5]decane-2,4-dione;
(5R,7S)-(5S,7R)-1-(3-fluorophenyl)-8-(3-isopropoxybenzyl)-7-methyl-1,3,8-triazaspiro[4.5]decane-2,4-dione;
1-(2-bromo-5-fluorophenyl)-8-(3-isopropoxybenzyl)-1,3,8-triazaspiro[4.5]decane-2,4-dione;
(5R,7S)-(5S,7R)-8-benzyl-1-(3-fluorophenyl)-7-methyl-1,3,8-triazaspiro[4.5]decane-2,4-dione;
N-(3-{4-fluoro-2-[8-(3-isopropoxybenzyl)-2,4-dioxo-1,3,8-triazaspiro[4.5]dec-1-yl]phenyl}prop-2-yn-1-yl)-N-methylmethanesulfonamide;
(5R,7S)-1-(3-fluorophenyl)-8-(3-isopropoxybenzyl)-7-methyl-1,3,8-triazaspiro[4.5]decane-2,4-dione;
(5R,7S)-(5S,7R)-8-benzyl-1-[4-fluoro-4′-(methylsulfonyl)biphenyl-2-yl]-7-methyl-1,3,8-triazaspiro[4.5]decane-2,4-dione;
(5R,7S)-1-(3-fluorophenyl)-7-methyl-8-[(2′-methylbiphenyl-3-yl)methyl]-1,3,8-triazaspiro[4.5]decane-2,4-dione;
1-(3-fluorophenyl)-8-[(2′-methylbiphenyl-3-yl)methyl]-3-(pyridin-2-ylmethyl)-1,3,8-triazaspiro[4.5]decan-2-one;
4′-fluoro-2′-[8-(3-isopropoxybenzyl)-2-oxo-1,3,8-triazaspiro[4.5]dec-1-yl)-N,N-dimethylbiphenyl-4-sulfonamide;
4′-fluoro-2′-[8-(3-isopropoxybenzyl)-2-oxo-3-propyl-1,3,8-triazaspiro[4.5]dec-1-yl]-N,N-dimethylbiphenyl-4-sulfonamide;
4′-fluoro-2′-[8-(3-isopropoxybenzyl)-3-methyl-2-oxo-1,3,8-triazaspiro[4.5]dec-1-yl]-N,N-dimethylbiphenyl-4-sulfonamide; 1-[4-fluoro-4′-(methylsulfonyl)biphenyl-2-yl]-8-(3-isopropoxybenzyl)-1,3,8-triazaspiro[4.5]decan-2-one;(5R,7S)-(5S,7R)-8-benzyl-1-(3-fluorophenyl)-7-methyl-1,3,8-triazaspiro[4.5]decan-2-one;(5R,7S)-(5S,7R)-1-(3-fluorophenyl)-7-methyl-8-(3-{[(1R)-1-methylpropyl]oxy}benzyl)-3-pent-4-en-1-yl-1,3,8-triazaspiro[4.5]decan-2-one;(5R,7S)-1-(3-fluorophenyl)-8-3-[(1R)-2-methoxy-1-methylethoxy]benzyl}-7-methyl-1,3,8-triazaspiro[4.5]decan-2-one;
(5R,7S)-(5S,7R)-8-benzyl-1-[4-fluoro-4′-(methylsulfonyl)-1,1′-biphenyl-2-yl]-7-methyl-1,3,8-triazaspiro[4.5]decane-2,4-dione;
(5R,7S)-1-(3-fluorophenyl)-7-methyl-8′-[(2′-methylbiphenyl-3-yl)methyl]-1,3,8-triazaspiro[4.5]decane-2,4-dione;
1-(3-fluorophenyl)-8-[(2′-methylbiphenyl-3-yl)methyl]-3-(pyridin-2-ylmethyl)-1,3,8-triazaspiro[4.5]decan-2-one;
(5R,7S)-8-benzyl-1-(3-fluorophenyl)-7-methyl-3-oxa-1,8-diazaspiro[4.5]decan-2-one; (5R,7S)-1-(3-fluorophenyl)-8-(3-isopropoxybenzyl)-3-[2-(4-methoxyphenyl)ethyl]-7-methyl-1,3,8-triazaspiro[4.5]decane-2,4-dione;
(5R,7S)-3-(cyclohexylmethyl)-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-3-(3-methoxybenzyl)-7-methyl-1,3,8-triazaspiro[4.5]decane-2,4-dione;
N-{2-[(5R,7S)-1-(3-fluorophenyl)-8-(3-isopropoxybenzyl)-7-methyl-2,4-dioxo-1,3,8-triazaspiro[4.5]dec-3-ylethyl}benzamide;
(5R,7S)-1-(3-fluorophenyl)-8-(3-isopropoxybenzyl)-7-methyl-3-[(1-methyl-1H-1,2,4-triazol-3-yl)methyl]-1,3,8-triazaspiro[4.5]decane-2,4-dione;
(5R,7S)-1-(3-fluorophenyl)-8-(3-isopropoxybenzyl)-7-methyl-3-[2-(1H-pyrazol-1-yl)ethyl]-1,3,8-triazaspiro[4.5]decane-2,4-dione;
(5R,7S)-1-(3-fluorophenyl)-8-(3-isopropoxybenzyl)-7-methyl-3-[(5-methyl-1,2,4-oxadiazol-3-yl)methyl]-1,3,8-triazaspiro[4.5]decane-2,4-dione;
(5R,7S)-3-(2-fluoroethyl)-1-(3-fluorophenyl)-8-(3-isopropoxybenzyl)-7-methyl-1,3,8-triazaspiro[4.5]decane-2,4-dione;
(5R,7S)-3-(1,2-benzisoxazol-3-ylmethyl)-1-(3-fluorophenyl)-8-(3-isopropoxybenzyl)-7-methyl-1,3,8-triazaspiro[4.5]decane-2,4-dione;
(5R,7S)-1-(3-fluorophenyl)-3-[2-(2-fluorophenyl)-2-oxoethyl]-8-(3-isopropoxybenzyl)-7-methyl-1,3,8-triazaspiro[4.5]decane-2,4-dione;
(5R,7S)-3-[(1-benzyl-1H-1,2,4-triazol-5-yl)methyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-3-(1H-imidazol-2-ylmethyl)-8-(3-isopropoxybenzyl)-7-methyl-1,3,8-triazaspiro[4.5]decane-2,4-dione;
(5R,7S)-3-{[5-(4-chlorophenyl)-1,3-oxazol-2-yl]methyl)}-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-3-[(3-phenyl-1,2,4-oxadiazol-5-yl)methyl]-1,3,8-triazaspiro[4.5]decane-2,4-dione;
(5R,7S)-3-[(5-cyclopropyl-1,3,4-thiadiazol-2-yl)methyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-3-[(5-methylisoxazol-3-yl)methyl]-1,3,8-triazaspiro[4.5]decane-2,4-dione;
(5R,7S)-1-(3-fluorophenyl)-8-(3-isopropoxybenzyl)-7-methyl-3-[(2-methyl-1,3-thiazol-4-yl)methyl]-1,3,8-triazaspiro[4.5]decane-2,4-dione;
(5R,7S)-1-(3-fluorophenyl)-8-(3-isopropoxybenzyl)-3-{[5-(4-methoxyphenyl)-1,2,4-oxadiazol-3-yl]methyl) -7-methyl-1,3,8-triazaspiro[4.5]decane-2,4-dione;
(5R,7S)-3-[(1,3-dimethyl-1H-pyrazol-5-yl)methyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-3-[(5-phenylisoxazol-3-yl)methyl]-1,3,8-triazaspiro[4.5]decane-2,4-dione;
(5R,7S)-3-{[5-(3,4-dichlorophenyl)isoxazol-3-yl]methyl)-1-(3-fluorophenyl)-8-(3-isopropoxybenzyl)-7-methyl-1,3,8-triazaspiro[4.5]decane-2,4-dione;
3-[(5R,7S)-1-(3-fluorophenyl)-8-(3-isopropoxybenzyl)-7-methyl-2,4-dioxo-1,3,8-triazaspiro[4.5]dec-3-yl]propanoic acid;
[(5R,7S)-1-(3-fluorophenyl)-8-(3-isopropoxybenzyl)-7-methyl-2,4-dioxo-1,3,8-triazaspiro[4.5]dec-3-yl]acetic acid;
N-(4-chlorophenyl)-2-[(5R,7S)-1-(3-fluorophenyl)-8-(3-isopropoxybenzyl)-7-methyl-2,4-dioxo-1,3,8-triazaspiro[4.5]dec-3-yl]acetamide;
(5R,7S)-1-(3-fluorophenyl)-8-(3-isopropoxybenzyl)-7-methyl-3-(1,2,4-oxadiazol-3-ylmethyl)-1,3,8-triazaspiro[4.5]decane-2,4-dione;
(5R,7S)-1-(3-fluorophenyl)-8-(3-isopropoxybenzyl)-7-methyl-3-(pyridin-2-ylmethyl)-1,3,8-triazaspiro[4.5]decane-2,4-dione;
(5R,7S)-1-(3-fluorophenyl)-3-(3-furylmethyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-3-(isoxazol-3-ylmethyl)-7-methyl-1,3,8-triazaspiro[4.5]decane-2,4-dione;
(5R,7S)-1-(3-fluorophenyl)-8-(3-isopropoxybenzyl)-7-methyl-3-(1,3-oxazol-2-ylmethyl)-1,3,8-triazaspiro[4.5]decane-2,4-dione;
(5R,7S)-1-(3-fluorophenyl)-8-(3-isopropoxybenzyl)-7-methyl-3-[(3-methyl-1,2,4-oxadiazol-5-yl)methyl]-1,3,8-triazaspiro[4.5]decane-2,4-dione;
(5R,7S)-3-[(3-ethyl-1,2,4-oxadiazol-5-yl)methyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-3-(isoxazol-5-ylmethyl)-7-methyl-1,3,8-triazaspiro[4.5]decane-2,4-dione;
(5R,7S)-1-(3-fluorophenyl)-8-(3-isopropoxybenzyl)-7-methyl-3-[(1-methyl-1H-1,2,4-triazol-5-yl)methyl]-1,3,8-triazaspiro[4.5]decane-2,4-dione;
(5R,7S)-1-(3-fluorophenyl)-8-(3-isopropoxybenzyl)-7-methyl-3-[(1-methyl-1H-imidazol-1-yl)methyl]-1,3,8-triazaspiro[4.5]decane-2,4-dione;
(5R,7S)-1-(3-fluorophenyl)-8-(3-isopropoxybenzyl)-7-methyl-3-[(1-phenyl-1H-1,2,3-triazol-4-yl)methyl]-1,3,8-triazaspiro[4.5]decane-2,4-dione;
(5R,7S)-1-(3-fluorophenyl)-8-(3-isopropoxybenzyl)-7-methyl-3-(pyrazin-2-ylmethyl)-1,3,8-triazaspiro[4.5]decane-2,4-dione;
(5R,7S)-3-tert-butyl-1-(3-fluorophenyl)-8-(3-isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-3-(1-methyl-1-phenylethyl)-1,3,8-triazaspiro[4.5]decane-2,4-dione;
(5R,7S)-1-(3-fluorophenyl)-8-(3-isopropoxybenzyl)-7-methyl-3-(1,3-thiazol-2-ylmethyl)-1,3,8-triazaspiro[4.5]decane-2,4-dione;
(5R,7S)-1-(3-fluorophenyl)-8-(3-isopropoxybenzyl)-7-methyl-3-(1,3-thiazol-4-ylmethyl)-1,3,8-triazaspiro[4.5]decane-2,4-dione;
(5R,7S)-1-(3-fluorophenyl)-8-(3-isopropoxybenzyl)-3-(2-methoxy-1,1-dimethylethyl)-7-methyl-1,3,8-triazaspiro[4.5]decane-2,4-dione;
(5R,7S)-1-(3-fluorophenyl)-3-(2-furylmethyl)-8-(3_{T}isopropoxybenzyl)-7-methyl-1,3,8-triazaspiro[4.5]decane-2,4-dione;
(5R,7S)-1-(3-fluorophenyl)-8-(3-isopropoxybenzyl)-7-methyl-2,4-dioxo-3-phenyl-1,3-diaza-8-azoniaspiro[4.5]decane;
(5R,7S)-1-(3-fluorophenyl)-8-(3-isopropoxybenzyl)-7-methyl-3-phenyl-1,3,8-triazaspiro[4.5]decane-2,4-dione;
(5R,7S)-1,3-bis(3-fluorophenyl)-8-(3-isopropoxybenzyl)-7-methyl-1,3,8-triazaspiro[4.5]decane-2,4-dione;
3-[(5R,7S)-1-(3-fluorophenyl)-8-(3-isopropoxybenzyl)-7-methyl-2,4-dioxo-1,3,8-triazaspiro[4.5]dec-3-yl]benzonitrile;
(5R,7S)-3-[3-(dimethylamino)phenyl]-1-(3-fluorophenyl)-8-(3-isopropoxybenzyl)-7-methyl-1,3,8-triazaspiro[4.5]decane-2,4-dione;
(5R,7S)-1-(3-fluorophenyl)-3-(1H-indol-5-yl)-8-(3-isopropoxybenzyl)-7-methyl-1,3,8-triazaspiro[4.5]decane-2,4-dione;
3-[(5R,7S)-1-(3-fluorophenyl)-8-(3-isopropoxybenzyl)-7-methyl-2,4-dioxo-1,3,8-triazaspiro[4.5]dec-3-yl]benzoic acid;
(5R,7S)-1-(3-fluorophenyl)-8-(3-furylmethyl)-7-methyl-3-[(5-methylisoxazol-3-yl)methyl]-1,3,8-triazaspiro[4.5]decane-2,4-dione;
N-[4-({(5R,7S)-1-(3-fluorophenyl)-7-methyl-3-[(5-methylisoxazol-3-yl)methyl]-2,4-dioxo-1,3,8-triazaspiro[4.5]dec-8-yl) methyl)phenyl]acetamide;
(5R,7S)-1-(3-fluorophenyl)-7-methyl-3-[(5-methylisoxazol-3-yl)methyl]-8-(pyridin-3-ylmethyl)-1,3,8-triazaspiro[4.5]decane-2,4-dione;
(5R,7S)-8-benzyl-1-(3-fluorophenyl)-7-methyl-3-[(5-methylisoxazol-3-yl)methyl]-1,3,8-triazaspiro[4.5]decane-2,4-dione;
(5R,7S)-8-(2-fluorobenzyl)-1-(3-fluorophenyl)-7-methyl-3-[(5-methylisoxazol-3-yl)methyl]-1,3,8-triazaspiro[4.5]decane-2,4-dione;
(5R,7S)-8-(cyclobutylmethyl)-1-(3-fluorophenyl)-7-methyl-3-[(5-methylisoxazol-3-yl)methyl]-1,3,8-triazaspiro[4.5]decane-2,4-dione;
(5R,7S)-8-benzyl-3-{[5-(3,4-dichlorophenyl)isoxazol-3-yl]methyl)-1-(3-fluorophenyl)-7-methyl-1,3,8-triazaspiro[4.5]decane-2,4-dione;
N-(4-{[(5R,7S)-1-(3-fluorophenyl)-7-methyl-2,4-dioxo-1,3,8-triazaspiro[4.5]dec-8-yl]methyl}phenyl)acetamide;
(5R,7S)-3-{[5-(3,4-dichlorophenyl)isoxazol-3-yl]methyl}-8-(2-fluorobenzyl)-1-(3-fluorophenyl)-7-methyl-1,3,8-triazaspiro[4.5]decane-2,4-dione;
(5R,7S)-8-(cyclobutylmethyl)-3-{[5-(3,4-dichlorophenyl)isoxazol-3-yl]methyl}-1-(3-fluorophenyl)-7-methyl-1,3,8-triazaspiro[4.5]decane-2,4-dione;
3-[(5R,7S)-8-(3-isopropoxybenzyl)-7-methyl-2,4-dioxo-1,3,8-triazaspiro[4.5]dec-1-yl]benzonitrile;
3-{(5R,7S)-8-(3-isopropoxybenzyl)-7-methyl-3-[(5-methylisoxazol-3-yl)methyl]-2,4-dioxo-1,3,8-triazaspiro[4.5]dec-1-yl)benzonitrile;
3-[(5R,7S)-3-[(5-cyclopropyl-1,3,4-thiadiazol-2-yl)methyl]-8-(3-isopropoxybenzyl)-7-methyl-2,4-dioxo-1,3,8-triazaspiro[4.5]dec-1-yl]benzonitrile;
3-[(5R,7S)-3-{[5-(3,4-dichlorophenyl)isoxazol-3-yl]methyl}-8-(3-isopropoxybenzyl)-7-methyl-2,4-dioxo-1,3,8-triazaspiro[4.5]dec-1-yl]benzonitrile;
3-{(5R,7S)-8-(3-isopropoxybenzyl)-7-methyl-2,4-dioxo-3-[(1-phenyl-1H-1,2,3-triazol-4-yl)methyl]-1,3,8-triazaspiro[4.5]dec-1-yl)benzonitrile;
3-{(5R,7S)-8-(3-isopropoxybenzyl)-3-[2-methoxyphenyl)ethyl]-7-methyl-2,4-dioxo-1,3,8-triazaspiro[4.5]dec-1-yl)benzonitrile;
N-{2-[(5R,7S)-1-(3-cyanophenyl)-8-(3-isopropoxybenzyl)-7-methyl-2,4-dioxo-1,3,8-triazaspiro[4.5]dec-3-yl]ethyl}benzamide;
3-((5R,7S)-8-(3-isopropoxybenzyl)-3-{[5-(4-methoxyphenyl)isoxazol-3-yl]methyl}-7-methyl-2,4-dioxo-1,3,8-triazaspiro[4.5]dec-1-yl)benzonitrile; and
(5R,7S)-1-(3-fluorophenyl)-7-methyl-8-[(2′-methylbiphenyl-3-yl)methyl]-3-[(5-methylisoxazol-3-yl)methyl]-1,3,8-triazaspiro[4.5]decane-2,4-dione;
and pharmaceutically acceptable salts thereof, and individual enantiomers and diastereomers thereof.

14. A pharmaceutical composition comprising a therapeutically effective amount of a compound of claim 1, or a pharmaceutically acceptable salt thereof, and a pharmaceutically acceptable carrier.

15. A method of treating Alzheimer's disease in a patient in need thereof, comprising administering to the patient a therapeutically effective amount of a compound of claim 1, or a pharmaceutically acceptable salt thereof, and a pharmaceutically acceptable carrier.

16. A method for the manufacture of a medicament for inhibiting β-secretase enzyme activity in mammals, comprising combining a therapeutically effective amount of a compound of claim 1, or a pharmaceutically acceptable salt thereof, and a pharmaceutically acceptable carrier.
